(19) United States
(12) Patent Application Publication
(10) Pub. No.: US 2021/0238170 A1
(43) Pub. Date: Aug. 5, 2021

(54) 5,5-Difluoro- and 5-Fluoro-5-Methyl-C-Glycoside Derivatives Useful As Dual SGLT1 / SGLT2 Modulators

(71) Applicant: Janssen Pharmaceutica NV, Beerse (BE)

(72) Inventors: Guozhang Xu, Chesterbrook, PA (US); Gee-Hong Kuo, Scotch Plains, NJ (US); Michael Gaul, Apex, NC (US)

(21) Appl. No.: 17/053,363

(22) PCT Filed: May 8, 2019
(86) PCT No.: PCT/IB2019/053778

§ 371 Date: Nov. 6, 2020

Related U.S. Application Data
(60) Provisional application No. 62/668,998, filed on May 9, 2018.

(51) Int. Cl. C07D 407/10; C07D 409/10; C07D 309/10; A61K 9/00; A61K 9/48; A61P 3/10

(52) U.S. Cl. CPC C07D 407/10; C07D 409/10; A61P 3/10; A61K 9/0053; A61K 9/48; C07D 309/10

(57) ABSTRACT

The present invention is directed to 5,5-difluoro- and 5-fluoro-5-methyl-C-glycoside derivatives, pharmaceutical compositions containing them and their use in the treatment of disorders and conditions modulated by SGLT activity, more particularly dual SGLT1/2 activity.

### FIELD OF THE INVENTION

The present invention is directed to 5,5-difluoro- and 5-fluoro-5-methyl-C-glycoside derivatives, pharmaceutical compositions containing them and their use in the treatment of disorders and conditions modulated by SGLT activity, more particularly dual SGLT1/2 activity. More particularly, the compounds of the present invention are useful in the treatment of for example, Type II diabetes mellitus, Syndrome X, and complications and symptoms associated with said disorders.

### BACKGROUND OF THE INVENTION

Diabetes is a chronic disorder affecting carbohydrate, fat and protein metabolism in animals.

Type I diabetes mellitus, which comprises approximately 10% of all diabetes cases, was previously referred to as insulin-dependent diabetes mellitus (IDDM) or juvenile onset diabetes. This disease is characterized by a progressive loss of insulin secretory function by beta cells of the pancreas. This characteristic is also shared by non-idiopathic, or “secondary”, diabetes having its origins in pancreatic disease. Type I diabetes mellitus is associated with the following clinical signs or symptoms: persistently elevated plasma glucose concentration or hyperglycemia; polyuria; polydipsia and/or hyperphagia; chronic microvascular complications such as retinopathy, nephropathy and neuropathy; and macrovascular complications such as hyperlipidemia and hypertension which can lead to blindness, end-stage renal disease, limb amputation and myocardial infarction. Therapy for IDDM patients has consistently focused on administration of exogenous insulin, which may be derived from various sources (e.g., human, bovine, porcine insulin). The use of heterologous species material gives rise to formation of anti-insulin antibodies which have activity limiting effects and result in progressive requirements for larger doses in order to achieve desired hypoglycemic effects.

Type II diabetes mellitus (non-insulin-dependent diabetes mellitus or NIDDM) is a metabolic disorder involving the dysregulation of glucose metabolism and impaired insulin sensitivity. Type II diabetes mellitus usually develops in adulthood and is associated with the body's inability to utilize or make sufficient insulin. In addition to the insulin resistance observed in the target tissues, patients suffering from type II diabetes mellitus have a relative insulin deficiency—that is, patients have lower than predicted insulin levels for a given plasma glucose concentration. Type II diabetes mellitus is characterized by the following clinical signs or symptoms: persistently elevated plasma glucose concentration or hyperglycemia; polyuria; polydipsia and/or hyperphagia; chronic microvascular complications such as retinopathy, nephropathy and neuropathy; and macrovascular complications such as hyperlipidemia and hypertension which can lead to blindness, end-stage renal disease, limb amputation and myocardial infarction. Typical treatment of Type II diabetes mellitus focuses on maintaining the blood glucose level as near to normal as possible with lifestyle modification relating to diet and exercise, and when necessary, the treatment with antidiabetic agents, insulin or a combination thereof. NIDDM that cannot be controlled by dietary management is treated with oral antidiabetic agents.

Syndrome X, also termed Insulin Resistance Syndrome (IRS), Metabolic Syndrome, or Metabolic Syndrome X, is recognized in some 2% of diagnostic coronary catheterizations. Often disabling, it presents symptoms or risk factors for the development of Type II diabetes mellitus and cardiovascular disease, including impaired glucose tolerance (IGT), impaired fasting glucose (IFG), hyperinsulinemia, insulin resistance, dyslipidemia (e.g., high triglycerides, low HDL), hypertension and obesity. Although insulin resistance is not always treated in all Syndrome X patients, those who exhibit a prediabetic state (e.g., IGT, IFG), where fasting glucose levels may be higher than normal but not at the diabetes diagnostic criterion, is treated in some countries (e.g., Germany) with metformin to prevent diabetes. The anti-diabetic agents may be combined with pharmacological agents for the treatment of the concomitant co-morbidities (e.g., antihypertensives for hypertension, hypolipidemic agents for lipidemia).

Hyperglycemia is one common characteristic of these diabetic disorders. Treatments of hyperglycemia are focused on excretion of excessive glucose directly into urine, which involves sodium-glucose cotransporters (SGLTs), primarily found in the chorionic membrane of the intestine and kidney. In particular, renal reabsorption of glucose is mediated by SGLT1 and SGLT2 (MARSENIC, O., “Glucose Control by the Kidney: An Emerging Target in Diabetes”, AM. J. Kidney Dis., 2009 May, pp 875-883, Vol. 53(5); WRIGHT, E. M., et al., “Biology of Human Sodium Glucose Transporters”, Physiol. Rev., 2011 April, pp 733-794, Vol. 91(2)). SGLT1, a high-affinity low-capacity transporter with a Na⁺:glucose transport ratio of 2:1, is present in intestinal and renal epithelial cells (LEE, W. S., et al., “The High Affinity Na+/Glucose Cotransporter. Re-evaluation of Function and Distribution of Expression”, J. Biol. Chem., 1994 April 22, pp 12032-12039, Vol. 269(16)). On the other hand, SGLT2, also known as SAAT1, a low-affinity high-capacity transporter with a Na⁺:glucose transport ratio of 1:1, is found in the epithelium of the kidney (YOU, G., et al., “Molecular Characteristic of Na(+)-coupled Glucose Transporters in Adult and Embryonic Rat Kidney”, J. Biol. Chem., 1995 Dec. 8, pp 29365-29371, Vol. 270(49); CHEN, J., et al., “Quantitative PCR Tissue Expression Profiling of the Human SGLT2 Gene and Related Family Members”, Diabetes Ther., 2010 December, pp 57-92, Vol. 1(2)). In addition, glucose absorption in the intestine is primarily mediated by SGLT1 and SGLT2. Thus, inhibition of SGLT1 and SGLT2 reduces plasma glucose through suppression of glucose reabsorption in the kidney, which was demonstrated in rodent models of IDDM and NIDDM by increasing the excretion of glucose in urine and lowering blood glucose levels.

Non-alcoholic fatty liver disease (NAFLD) is one cause of a fatty liver, occurring when fat is deposited (steatosis) in the liver. NAFLD is considered to cover a spectrum of disease activity. This spectrum begins as fatty accumulation in the liver (hepatic steatosis). A liver can remain fatty without disturbing liver function, but by varying mechanisms and possible insults to the liver may also progress to become NASH, a state in which steatosis is combined with inflammation and fibrosis. Non-alcoholic steatohepatitis (NASH) is a progressive, severe form of NAFLD. Over a 10-year period, up to 20% of patients with NASH will develop cirrhosis of the liver, and 10% will suffer death related to liver disease. The exact cause of NAFLD is still unknown, however, both obesity and insulin resistance are thought to play a strong role in the disease process. The exact reasons and mechanisms by which the disease progresses from one stage to the next are not known.

NAFLD has been linked to insulin resistance (IR) and the metabolic syndrome (MS). As the renin-angiotensin system (RAS) plays a central role in insulin resistance, and subsequently in NAFLD and NASH, an attempt to block the deleterious effects of RAS overexpression has been proposed a target for treatment. While many potential therapies tested in NASH target only the consequences of this condition, or try to “get rid” of excessive fat, angiotensin receptor blockers (ARBs) may act as a tool for correction of the various imbalances that act in harmony in NASH/NAFLD. Indeed, by inhibiting RAS the intracellular insulin signaling pathway may be improved, resulting in better control of adipose tissue proliferation and adipokine production, as well as more balanced local and systemic levels of various cytokines. At the same time, by controlling the local RAS in the liver fibrosis may be prevented and the cycle that links steatosis to necroinflammation slowed down. (GEORGESCU, E. F., in Advances in Therapy, 2008, pp 1141-1174, Vol. 25, Issue 11)

SCAFOGLIO, C., et al., in “Functional expression of sodium-glucose transporters in cancer”, PNAS, 2015, pp E41111-E4119, Vol 112(3), describe the role of sodium-dependent glucose transporters (SGLTs) in pancreatic and prostate adenocarcinomas, and their role in cancer cell survival. SGLT2 was found to be functionally expressed in pancreatic and prostate adenocarcinomas and further found to block glucose uptake and reduce tumor growth and survival in a xenograft model of pancreatic cancer, suggesting that SGLT2 inhibitors could be useful in treating certain types of cancers.

FRICK, W., et al., in U.S. Pat. No. 7,288,528, issued Oct. 30, 2007 describe aromatic fluoroglycoside derivates, medicaments containing these compound, and the use thereof.

FRICK W., et al., in US Patent Publication 2011/0059910 A1, published Mar. 10, 2011 describe substituted aromatic fluoroglycoside derivatives and physiologically compatible salts and physiologically functional derivatives thereof. Frick, w., et al., further describe methods of lowering blood sugar and the treatment of Type I and Type II diabetes.

CHEN, Z-H., et al., in “Synthesis and biological evaluation of SGLT2 inhibitors: gem-difluoromethylenated Dapagliflozin analogs”, Tetrahedron Letters, 2012, pp 2171-2176, Vol. 53(17) describe the synthesis and biological evaluation of SGLT2 gem-difluoromethylenated Dapagliflozin analogs.

WRIGHT E. M., et a., in US Patent Publication 2014/027147, published Sep. 18, 2014 describe radiolabeled traces for binding to sodium/glucose cotransporters (SGLTs) and their synthesis. Wright et al., further describe in vivo and in vitro techniques for using the tracers as analystic tools to study the biodistribution and regulation of SGLTs in health and disease, and to evaluate therapeutic interventions.

There remains a need for SGLT inhibitor compounds, more particularly dual SGLT1/2 inhibitor compounds, that have pharmacokinetic and pharmacodynamic properties suitable for use as human pharmaceuticals for the treatment of, for example, Type II Diabetes Mellitus.

### SUMMARY OF THE INVENTION

The present invention is directed to compounds of formula (I)

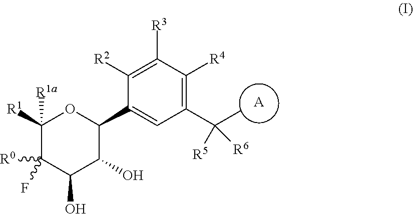

wherein

R⁰ is selected from the group consisting of fluoro and C₁₋₄alkyl;

R¹ is hydroxy substituted C₁₋₄alkyl;

R^{1a} is hydrogen;

alternatively, R¹ and R^{1a} are taken together with the carbon atom to which they are bound to form cycloprop-1,1-diyl;

R² is selected from the group consisting of hydrogen, halogen, hydroxy, C₁₋₄alkyl, —(C₁₋₄alkyl)-OH, C₁₋₄alkoxy, cyano substituted C₁₋₄alkoxy, —(C₁₋₂ alkoxy)-(C₁₋₂alkoxy), C₂₋₄alkenyl, C₂₋₄alkenyl-oxy, benzyloxy and carboxy;

R³ is selected from the group consisting of hydrogen, halogen, C₁₋₄alkyl and C₂₋₄alkenyl;

R⁴ is selected from the group consisting of hydrogen, halogen, hydroxy, cyano, C₁₋₄alkyl, fluorinated C₁₋₂alkyl, C₁₋₄alkoxy, fluorinated C₁₋₂alkoxy, cyano substituted C₁₋₄alkoxy and C₃₋₆cycloalkyl;

alternatively, R² and R³ or R³ and R⁴ are taken together with the carbon atoms to which they are bound to form 2,3-dihydrofuranyl; wherein —R²—R³— is selected from the group consisting of —O—CH₂—CH₂— and —CH₂—CH₂—O; and wherein —R³—R⁴— is selected from the group consisting of —O—CH₂—CH₂— and —CH₂—CH₂—O—;

wherein the 2,3-dihydrofuranyl is optionally substituted on any of its carbon atoms with one or more substituents independently selected from the group consisting of hydroxy, methyl, ethyl, hydroxymethyl- and hydroxyethyl-;

R⁵ and R⁶ are the same and are each hydrogen;

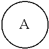

is selected from the group consisting of C₅₋₁₂cycloalkyl, C₅₋₁₂cycloalkenyl, phenyl, heteroaryl and heterocyclyl;

wherein the phenyl, heteroaryl or heterocyclyl is optionally substituted with one or more substituents independently selected from the group consisting of halogen, hydroxy, oxo (i.e. ═O), C₁₋₄alkyl, C₁₋₄alkoxy, fluorinated C₁₋₂alkyl, fluorinated C₁₋₂alkoxy, cyano, C₃₋₆cycloalkyl, tetrahydrofuranyl, phenyl, pyrid-2-yl, fluoro-substituted phenyl, fluoro-substituted pyrid-2-yl, —C(O)—R¹¹ and —NR¹²R¹³;

wherein R¹¹ is selected from the group consisting of C₁₋₄alkyl, C₁₋₄alkoxy, C₃₋₆cycloalkyl, azetidin-1-yl, pyrrolidin-1-yl, piperidin-1-yl, piperazin-1-yl, morpholin-4-yl, thiomorpholin-4-yl, thiazol-2-yl and 5-methyl-thiazol-2-yl;

wherein R¹² and R¹³ are each independently selected from the group consisting of hydrogen and C₁₋₄alkyl;

provided that when R⁰ is fluoro, R¹ is —CH₂OH, R^{1a} is hydrogen, R² is hydrogen, R³ is hydrogen, R⁴ is selected from the group consisting of chloro, fluoro, methyl, methoxy, trifluoromethyl and trifluoromethoxy, R⁵ is hydrogen and R⁶ is hydrogen,

then

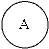

is other than phenyl, pyrid-2-yl or pyrid-3-yl; wherein the phenyl is substituted at the 4-position, the pyrid-2-yl is substituted at the 4- or 5-position and wherein the pyrid-3-yl is substituted at the 5-position with a substituent selected from the group consisting of halogen, C₁₋₄alkyl, C₁₋₄alkoxy, trifluoromethyl and trifluoromethoxy;

and isotopologues and pharmaceutically acceptable salts thereof.

The present invention is further directed to processes for the preparation of the compounds of formula (I). The present invention is further directed to a compound prepared according to any of the process(es) described herein.

Illustrative of the invention are pharmaceutical compositions comprising a pharmaceutically acceptable carrier and a compound of formula (I) as described herein. An illustration of the invention is a pharmaceutical composition made by mixing a compound of formula (I) as described herein and a pharmaceutically acceptable carrier. Illustrating the invention is a process for making a pharmaceutical composition comprising mixing a compound of formula (I) as described herein and a pharmaceutically acceptable carrier.

Exemplifying the invention are methods of treating a disease, disorder, or condition mediated by SGLT activity (preferably, dual SGLT1 and SGLT2 activity) such as impaired glucose tolerance (IGT), impaired fasting glucose (IFT), gestational diabetes, Type II diabetes mellitus, Syndrome X (also known as Metabolic Syndrome), obesity, nephropathy, neuropathy, retinopathy, hypertension, angina, atherosclerosis, heart disease, heart attack, ischemia, stroke, nerve damage or poor blood flow in the feet, non-alcoholic steatohepatitis (NASH), non-alcoholic fatty liver disease (NAFLD), liver fibrosis, cataracts, polycystic ovarian syndrome, irritable bowel syndrome, inflammation or cancer (preferably prostate cancer or pancreatic cancer), comprising administering to a subject in need thereof a therapeutically effective amount of any of the compounds or pharmaceutical compositions described above.

In an embodiment, the present invention is directed to a compound of formula (I) for use as a medicament. In another embodiment, the present invention is directed to a compound of formula (I) for use in the treatment of a disorder mediated SGLT activity (preferably dual SGLT1 and SGLT2 activity) such as impaired glucose tolerance (IGT), impaired fasting glucose (IFT), gestational diabetes, Type II diabetes mellitus, Syndrome X (also known as Metabolic Syndrome), obesity, nephropathy, neuropathy, retinopathy, hypertension, angina, atherosclerosis, heart disease, heart attack, ischemia, stroke, nerve damage or poor blood flow in the feet, non-alcoholic steatohepatitis (NASH), non-alcoholic fatty liver disease (NAFLD), liver fibrosis, cataracts, polycystic ovarian syndrome, irritable bowel syndrome, inflammation or cancer (preferably prostate cancer or pancreatic cancer). In another embodiment, the present invention is directed to a composition comprising a compound of formula (I) for the treatment of a disorder mediated by SGLT activity (preferably dual SGLT1 and SGLT2 activity) such as impaired glucose tolerance (IGT), impaired fasting glucose (IFT), gestational diabetes, Type II diabetes mellitus, Syndrome X (also known as Metabolic Syndrome), obesity, nephropathy, neuropathy, retinopathy, hypertension, angina, atherosclerosis, heart disease, heart attack, ischemia, stroke, nerve damage or poor blood flow in the feet, non-alcoholic steatohepatitis (NASH), non-alcoholic fatty liver disease (NAFLD), liver fibrosis, cataracts, polycystic ovarian syndrome, irritable bowel syndrome, inflammation or cancer (preferably prostate cancer or pancreatic cancer).

Another example of the invention is the use of any of the compounds described herein in the preparation of a medicament for treating: (a) impaired glucose tolerance (IGT), (b) impaired fasting glucose (IFT), (c) gestational diabetes, (d) Type II diabetes mellitus, (e) Syndrome X (also known as Metabolic Syndrome), (f) obesity, (g) nephropathy, (h) neuropathy, (i) retinopathy, (j) hypertension, (k) angina, (l) atherosclerosis, (m) heart disease, (n) heart attack, (o) ischemia, (p) stroke, (q) nerve damage or poor blood flow in the feet, (r) non-alcoholic steatohepatitis (NASH), (s) non-alcoholic fatty liver disease (NAFLD), (t) liver fibrosis, (u) cataracts, (v) polycystic ovarian syndrome, (w) irritable bowel syndrome, (x) inflammation and (y) cancer (preferably prostate cancer or pancreatic cancer), in a subject in need thereof. In another example, the present invention is directed to a compound as described herein for use in a methods for treating a disorder selected from the group consisting of impaired glucose tolerance (IGT), impaired fasting glucose (IFT), gestational diabetes, Type II diabetes mellitus, Syndrome X (also known as Metabolic Syndrome), obesity, nephropathy, neuropathy, retinopathy, hypertension, angina, atherosclerosis, heart disease, heart attack, ischemia, stroke, nerve damage or poor blood flow in the feet, non-alcoholic steatohepatitis (NASH), non-alcoholic fatty liver disease (NAFLD), liver fibrosis, cataracts, polycystic ovarian syndrome, irritable bowel syndrome, inflammation and cancer (preferably prostate cancer or pancreatic cancer), in a subject in need thereof.

### DETAILED DESCRIPTION OF THE INVENTION

The present invention is directed to compounds of formula (I)

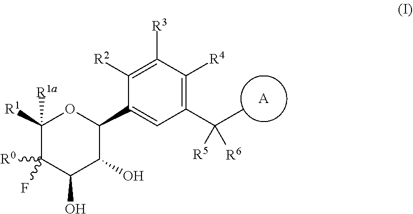

wherein R⁰, R¹, R^{1a}, R², R³, R⁴, R⁵, R⁶ and

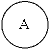

are as herein defined, and isotopologues and pharmaceutically acceptable salts thereof. The compounds of the present invention are useful in the treatment of diseases, disorders and complications associated with SGLT activity (preferably dual SGLT1 and SGLT2 activity) selected from the group of impaired glucose tolerance (IGT), impaired fasting glucose (IFT), gestational diabetes, Type II diabetes mellitus, Syndrome X (also known as Metabolic Syndrome), obesity, nephropathy, neuropathy, retinopathy, hypertension, angina, atherosclerosis, heart disease, heart attack, ischemia, stroke, nerve damage or poor blood flow in the feet, non-alcoholic steatohepatitis (NASH), non-alcoholic fatty liver disease (NAFLD), liver fibrosis, cataracts, polycystic ovarian syndrome, irritable bowel syndrome, inflammation and cancer (preferably prostate cancer or pancreatic cancer).

In certain embodiments, the present invention is directed to compounds of formula (I) wherein R⁰ is selected from the group consisting of fluoro and C₁₋₄ alkyl. In certain embodiments, the present invention is directed to compounds of formula (I) wherein R⁰ is selected from the group consisting of fluoro and C₁₋₂ alkyl.

In certain embodiments, the present invention is directed to compounds of formula (I) wherein R⁰ is selected from the group consisting of fluoro and methyl. In certain embodiments, the present invention is directed to compounds of formula (I) wherein R⁰ is methyl. In certain embodiments, the present invention is directed to compounds of formula (I) wherein R⁰ is fluoro.

In certain embodiments, the present invention is directed to compounds of formula (I) wherein R¹ is hydroxy substituted C₁₋₄alkyl. In certain embodiments, the present invention is directed to compounds of formula (I) wherein R¹ is hydroxy substituted C₁₋₂alkyl. In certain embodiments, the present invention is directed to compounds of formula (I) wherein R¹ is hydroxymethyl-.

In certain embodiments, the present invention is directed to compounds of formula (I) wherein R^{1a} is hydrogen.

In certain embodiments, the present invention is directed to compounds of formula (I) wherein R¹ and R^{1a} are taken together with the carbon atom to which they are bound to form cycloprop-1,1-diyl.

In certain embodiments, the present invention is directed to compounds of formula (I) wherein R² is selected from the group consisting of hydrogen, halogen, hydroxy, C₁₋₄alkyl, —(C₁₋₄alkyl)-OH, C₁₋₄alkoxy, cyano substituted C₁₋₄ alkoxy, —(C₁₋₂alkoxy)-(C₁₋₂alkoxy), and carboxy. In certain embodiments, the present invention is directed to compounds of formula (I) wherein R² is selected from the group consisting of hydrogen, hydroxy, C₁₋₄alkoxy, —(C₁₋₂alkoxy)-(C₁₋₂ alkoxy) and cyano substituted C₁₋₂alkoxy.

In certain embodiments, the present invention is directed to compounds of formula (I) wherein R² is selected from the group consisting of hydrogen, hydroxy, methoxy, ethoxy, isopropyloxy, methoxy-ethoxy- and cyano-methoxy-. In certain embodiments, the present invention is directed to compounds of formula (I) wherein R² is selected from the group consisting of hydrogen, hydroxy, methoxy and cyano-methoxy-. In certain embodiments, the present invention is directed to compounds of formula (I) wherein R² is selected from the group consisting of hydroxy, methoxy and cyano-methoxy-. In certain embodiments, the present invention is directed to compounds of formula (I) wherein R² is selected from the group consisting of hydrogen, hydroxy and methoxy. In certain embodiments, the present invention is directed to compounds of formula (I) wherein R² is selected from the group consisting of hydroxy and methoxy. In certain embodiments, the present invention is directed to compounds of formula (I) wherein R² is hydroxy.

In certain embodiments, the present invention is directed to compounds of formula (I) wherein R³ is selected from the group consisting of hydrogen, halogen and C₁₋₄alkyl. In certain embodiments, the present invention is directed to compounds of formula (I) wherein R³ is selected from the group consisting of hydrogen, halogen and C₁₋₂alkyl.

In certain embodiments, the present invention is directed to compounds of formula (I) wherein R³ is selected from the group consisting of hydrogen, bromo, iodo and methyl. In certain embodiments, the present invention is directed to compounds of formula (I) wherein R³ is selected from the group consisting of hydrogen, bromo and methyl. In certain embodiments, the present invention is directed to compounds of formula (I) wherein R³ is selected from the group consisting of hydrogen and iodo. In certain embodiments, the present invention is directed to compounds of formula (I) wherein R³ is selected from the group consisting of hydrogen and methyl. In certain embodiments, the present invention is directed to compounds of formula (I) wherein R³ is hydrogen.

In certain embodiments, the present invention is directed to compounds of formula (I) wherein R⁴ is selected from the group consisting of hydrogen, halogen, hydroxy, cyano, C₁₋₄alkyl, fluorinated C₁₋₂alkyl, C₁₋₄alkoxy, fluorinated C₁₋₂alkoxy and C₃₋₆cycloalkyl. In certain embodiments, the present invention is directed to compounds of formula (I) wherein R⁴ is selected from the group consisting of hydrogen, halogen, cyano, C₁₋₂alkyl, C₁₋₂alkoxy and C₃₋₅cycloalkyl.

In certain embodiments, the present invention is directed to compounds of formula (I) wherein R⁴ is selected from the group consisting of hydrogen, chloro, cyano, methyl, ethyl, methoxy and cyclopropyl. In certain embodiments, the present invention is directed to compounds of formula (I) wherein R⁴ is selected from the group consisting of hydrogen, chloro, methyl, ethyl, methoxy and cyclopropyl. In certain embodiments, the present invention is directed to compounds of formula (I) wherein R⁴ is selected from the group consisting of hydrogen, chloro, methyl, ethyl and methoxy. In certain embodiments, the present invention is directed to compounds of formula (I) wherein R⁴ is selected from the group consisting of chloro, methyl, ethyl and methoxy.

In certain embodiments, the present invention is directed to compounds of formula (I) wherein the

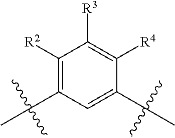

is selected from the group consisting of

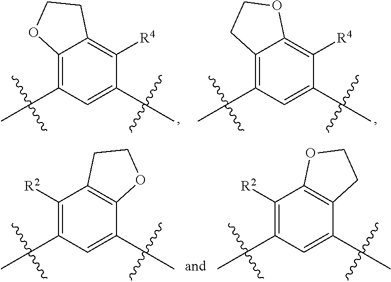

(such that —R²—R³— is selected from the group consisting of —O—CH₂—CH₂— and —CH₂—CH₂—O; and —R³—R⁴— is selected from the group consisting of —CH₂—CH₂—O— and —O—CH₂—CH₂—, respectively); wherein the 2,3-dihydrofuranyl portion of the structure is optionally substituted as herein defined.

In certain embodiments, the present invention is directed to compounds of formula (I) wherein the

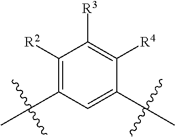

is selected from the group consisting of

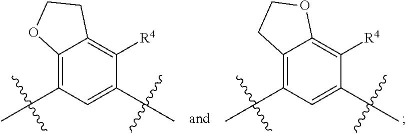

wherein the 2,3-dihydrofuranyl portion of the structure is optionally substituted as herein defined. In certain embodiments, the present invention is directed to compounds of formula (I) wherein the

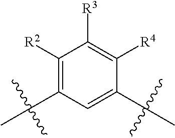

is selected from the group consisting of

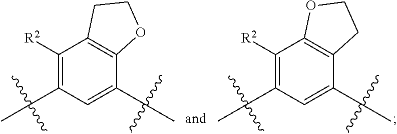

wherein the 2,3-dihydrofuranyl portion of the structure is optionally substituted as herein defined.

In certain embodiments, the present invention is directed to compounds of formula (I) wherein the

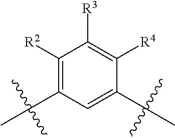

is selected from the group consisting of

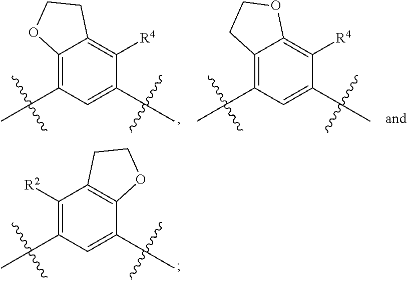

wherein the 2,3-dihydrofuranyl portion of the structure is optionally substituted as herein defined. In certain embodiments, the present invention is directed to compounds of formula (I) wherein the

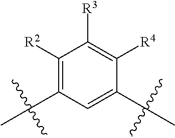

is selected from the group consisting of

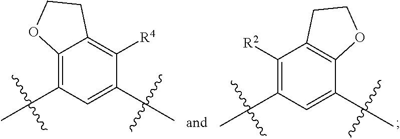

wherein the 2,3-dihydrofuranyl portion of the structure is optionally substituted as herein defined.

In certain embodiments, the present invention is directed to compounds of formula (I) wherein the

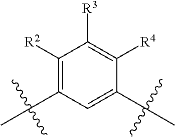

is

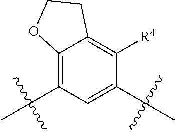

(such that —R²—R³— is —O—CH₂—CH₂—); wherein the 2,3-dihydrofuranyl portion of the structure is optionally substituted as herein defined. In certain embodiments, the present invention is directed to compounds of formula (I) wherein the

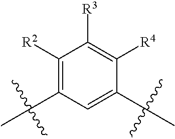

is

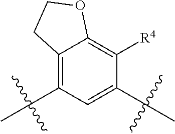

(such that —R²—R³— is —CH₂—CH₂—O—); wherein the 2,3-dihydrofuranyl portion of the structure is optionally substituted as herein defined. In certain embodiments, the present invention is directed to compounds of formula (I) wherein the

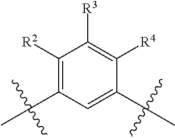

is

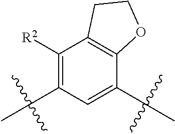

(such that —R³—R⁴— is —CH₂—CH₂—O—); wherein the 2,3-dihydrofuranyl portion of the structure is optionally substituted as herein defined. In certain embodiments, the present invention is directed to compounds of formula (I) wherein the

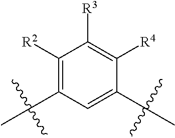

is

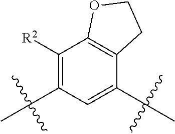

(such that —R³—R⁴— is —O—CH₂—CH₂—); wherein the 2,3-dihydrofuranyl portion of the structure is optionally substituted as herein defined.

In certain embodiments, the present invention is directed to compounds of formula (I) wherein R² and R³ or R³ and R⁴ are taken together with the carbon atoms to which they are bound to form 2,3-dihydrofuranyl; wherein —R²—R³— is selected from the group consisting of —O—CH₂—CH₂— and —CH₂—CH₂—O; wherein —R³—R⁴— is selected from the group consisting of —O—CH₂—CH₂— and —CH₂—CH₂—O—; wherein the 2,3-dihydrofuranyl is optionally substituted on any of its carbon atoms with one or more substituents independently selected from the group consisting of hydroxy, hydroxymethyl- and hydroxyethyl-. In certain embodiments, the present invention is directed to compounds of formula (I) wherein R² and R³ or R³ and R⁴ are taken together with the carbon atoms to which they are bound to form 2,3-dihydro-furanyl; wherein —R²—R³— is selected from the group consisting of —O—CH₂—CH₂— and —CH₂—CH₂—O; wherein —R³—R⁴— is selected from the group consisting of —O—CH₂—CH₂— and —CH₂—CH₂—O—; and wherein the 2,3-dihydrofuranyl is optionally substituted on any of its carbon atoms with one to two substituents independently selected from the group consisting of hydroxymethyl- and hydroxyethyl-.

In certain embodiments, the present invention is directed to compounds of formula (I) wherein R² and R³ or R³ and R⁴ are taken together with the carbon atoms to which they are bound to form a ring structure selected from the group consisting of 2,3-dihydro-furanyl and 3-(hydroxymethyl)-2,3-dihydrofuranyl wherein —R²—R³— is selected from the group consisting of —CH₂—CH₂—O— and —O—CH₂—CH₂—; and wherein —R³—R⁴ is selected from the group consisting of —CH₂—CH₂—O—, —CH(CH₂OH)—CH₂—CH₂—O— and —O—CH₂—CH₂—.

In certain embodiments, the present invention is directed to compounds of formula (I) wherein R² and R³ or R³ and R⁴ are taken together with the carbon atoms to which they are bound to form 2,3-dihydro-furanyl; wherein —R²—R³— is —O—CH₂—CH₂— and wherein —R³—R⁴— is selected from the group consisting of —CH₂—CH₂—O— and —O—CH₂—CH₂—.

In certain embodiments, the present invention is directed to compounds of formula (I) wherein R³ and R⁴ are taken together with the carbon atoms to which they are bound to form 2,3-dihydro-furanyl; wherein —R₃—R₄— is selected from the group consisting of —CH₂—CH₂—O and —O—CH₂—CH₂—. In certain embodiments, the present invention is directed to compounds of formula (I) wherein R³ and R⁴ are taken together with the carbon atoms to which they are bound to form 2,3-dihydro-furanyl; and wherein —R³—R⁴— is —CH₂—CH₂—O—;

In certain embodiments, the present invention is directed to compounds of formula (I) wherein R⁵ and R⁶ are the same and are selected from the group consisting of hydrogen and deuterium. In certain embodiments, the present invention is directed to compounds of formula (I) wherein R⁵ and R⁶ are the same and are each hydrogen. In certain embodiments, the present invention is directed to compounds of formula (I) wherein R⁵ and R⁶ are the same and are each deuterium.

In certain embodiments, the present invention is directed to compounds of formula (I) wherein

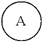

is selected from the group consisting of phenyl, heteroaryl and heterocyclyl; wherein the phenyl, heteroaryl or heterocyclyl is optionally substituted with one or more substituents independently selected from the group consisting of halogen, hydroxy, oxo, C₁₋₄alkyl, C₁₋₄alkoxy, fluorinated C₁₋₂alkyl, fluorinated C₁₋₂alkoxy, cyano, C₃₋₆cycloalkyl, tetrahydrofuranyl, phenyl, pyrid-2-yl, fluoro-substituted phenyl, fluoro-substituted pyrid-2-yl, —C(O)—R¹¹ and —NR¹²R¹³; wherein R¹¹ is selected from the group consisting of C₁₋₄alkyl, C₁₋₄alkoxy, C₃₋₆cycloalkyl, azetidin-1-yl, pyrrolidin-1-yl, piperidin-1-yl, piperazin-1-yl, thiazol-2-yl and 5-methyl-thiazol-2-yl; and wherein R¹² and R¹³ are each independently selected from the group consisting of hydrogen and C₁₋₄alkyl.

In certain embodiments, the present invention is directed to compounds of formula (I) wherein

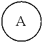

is selected from the group consisting of phenyl, thienyl, benzothienyl, 2,3-dihydrobenzofuranyl, chromanyl, 2H-benzo[b][1,4]oxazinyl, 2,3-dihydrobenzo[b][1,4]oxathiinyl, 6,7-dihydrothieno[3,2-c]pyridinyl and 2,3-dihydro-benzo[b][1,4]dioxin-6-yl; wherein the phenyl, thienyl, benzothienyl, 2,3-dihydrobenzofuranyl, chromanyl, 2H-benzo[b][1,4]oxazinyl, 2,3-dihydrobenzo[b][1,4]oxathiinyl, 6,7-dihydrothieno[3,2-c]pyridin-2-yl or 2,3-dihydro-benzo[b][1,4]dioxin-6-yl is optionally substituted with one to two substituents independently selected from the group consisting of halogen, oxo, cyano, C₁₋₂alkyl, C₁₋₂alkoxy, fluorinated C₁₋₂alkoxy, C₃₋₅cycloalkyl, tetrahydrofuranyl, phenyl, fluoro substituted phenyl, pyrid-2-yl, fluoro substituted pyrid-2-yl, —C(O)—R¹¹ and —NR¹²R¹³; wherein R¹¹ is selected from the group consisting of C₁₋₄alkyl, C₁₋₄alkoxy, C₃₋₆cycloalkyl, pyrrolidin-1-yl, thiazol-2-yl and 5-methyl-thiazol-2-yl; and wherein R¹² and R¹³ are each independently selected from the group consisting of hydrogen and C₁₋₂alkyl.

In certain embodiments, the present invention is directed to compounds of formula (I) wherein

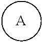

is selected from the group consisting of 4-chloro-phenyl, 3-fluoro-4-methyl-phenyl, 3-bromo-4-methoxy-phenyl, 4-methyl-phenyl, 4-ethyl-phenyl, 3-methoxy-phenyl, 4-methoxy-phenyl, 4-ethoxy-phenyl, 4-(fluoro-methoxy)-phenyl, 4-(difluoromethoxy)-phenyl, 4-(trifluoro-methoxy)-phenyl, 4-(dimethylamino)-phenyl, 4-(cyclopropyl)-phenyl, 4-((S)-tetrahydrofuran-3-yl)-phenyl), 5-chloro-thien-2-yl, 5-methyl-thien-2-yl, 4-ethyl-thien-2-yl, 5-(3-fluoro-phenyl)-thien-2-yl, 5-(4-fluoro-phenyl)-thien-2-yl, 5-(6-fluoro-pyrid-2-yl)-thien-2-yl, benzothien-2-yl, 5-fluoro-benzothien-2-yl, 2,3-dihydro-benzofuran-5-yl, 2,3-dihydro-benzofuran-6-yl, chroman-6-yl, 4-methyl-2H-benzo[b][1,4]oxazin-7-yl-3-one, 2,3-dihydrobenzo[b][1,4]oxathiin-6-yl, 2,3-dihydrobenzo[b][1,4]oxathiin-6-yl-4,4-dioxide, 5-cyano-6,7-dihydrothieno[3,2-c]pyridin-2-yl, 5-(methyl-carbonyl)-6,7-dihydrothieno[3,2-c]pyridin-2-yl, 5-(methoxy-carbonyl)-6,7-dihydrothieno[3,2-c]pyridin-2-yl, 5-(cyclopentyl-carbonyl)-6,7-dihydrothieno[3,2-c]pyridin-2-yl, 5-(pyrrolidin-1-yl-carbonyl)-6,7-dihydrothieno[3,2-c]pyridin-2-yl, 5-(thiazol-2-yl-carbonyl)-6,7-dihydrothieno[3,2-c]pyridin-2-yl, 5-(5-methyl-thiazol-2-yl-carbonyl)-6,7-dihydrothieno[3,2-c]pyridin-2-yl, and 2,3-dihydro-benzo[b][1,4]dioxin-6-yl.

In certain embodiments, the present invention is directed to compounds of formula (I) wherein

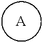

is selected from the group consisting of 4-chloro-phenyl, 3-fluoro-4-methyl-phenyl, 4-methyl-phenyl, 4-ethyl-phenyl, 3-methoxy-phenyl, 4-methoxy-phenyl, 4-ethoxy-phenyl, 4-(fluoro-methoxy)-phenyl, 4-(difluoromethoxy)-phenyl, 4-(cyclopropyl)-phenyl, 5-chloro-thien-2-yl, 5-methyl-thien-2-yl, 4-ethyl-thien-2-yl, 5-(6-fluoro-pyrid-2-yl)-thien-2-yl, benzothien-2-yl, 5-fluoro-benzothien-2-yl, 2,3-dihydro-benzofuran-5-yl, 2,3-dihydro-benzofuran-6-yl, chroman-6-yl, 2,3-dihydrobenzo[b][1,4]oxathiin-6-yl, 5-cyano-6,7-dihydrothieno[3,2-c]pyridin-2-yl, 5-(5-methyl-thiazol-2-yl-carbonyl)-6,7-dihydrothieno[3,2-c]pyridin-2-yl and 2,3-dihydro-benzo[b][1,4]dioxin-6-yl.

In certain embodiments, the present invention is directed to compounds of formula (I) wherein

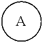

is selected from the group consisting of 4-chloro-phenyl, 3-fluoro-4-methyl-phenyl, 4-methyl-phenyl, 4-ethyl-phenyl, 3-methoxy-phenyl, 4-methoxy-phenyl, 4-ethoxy-phenyl, 4-(fluoro-methoxy)-phenyl, 4-(difluoromethoxy)-phenyl, 4-(cyclopropyl)-phenyl, 5-chloro-thien-2-yl, 5-methyl-thien-2-yl, 4-ethyl-thien-2-yl, 5-(6-fluoro-pyrid-2-yl)-thien-2-yl, benzothien-2-yl, 2,3-dihydro-benzofuran-6-yl, chroman-6-yl and 2,3-dihydro-benzo[b][1,4]dioxin-6-yl.

In certain embodiments, the present invention is directed to compounds of formula (I) wherein

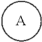

is selected from the group consisting of 4-chloro-phenyl, 4-methyl-phenyl, 4-methoxy-phenyl, 4-ethoxy-phenyl, 4-(fluoro-methoxy)-phenyl, 4-(cyclopropyl)-phenyl, 5-chloro-thien-2-yl, 5-methyl-thien-2-yl, 4-ethyl-thien-2-yl, benzothien-2-yl, 2,3-dihydro-benzofuran-6-yl, chroman-6-yl and 2,3-dihydro-benzo[b][1,4]dioxin-6-yl.

In certain embodiments, the present invention is directed to compounds of formula (I) wherein

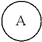

is selected from the group consisting of 4-methoxy-phenyl, 4-(fluoro-methoxy)-phenyl, benzothien-2-yl, 5-fluoro-benzothien-2-yl, chroman-6-yl and 2,3-dihydro-benzo[b][1,4]dioxin-6-yl.

In certain embodiments, the present invention is directed to compounds of formula (I) wherein

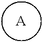

is selected from the group consisting of 4-methoxy-phenyl, 4-(fluoromethoxy)-phenyl, benzothien-2-yl and 2,3-dihydro-benzo[b][1,4]dioxin-6-yl.

In certain embodiment of the present invention,

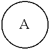

is other than optionally substituted phenyl. In certain embodiments of the present invention

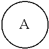

is other than optionally substituted pyrid-2-yl or pyrid-3-yl. In certain embodiments of the present invention

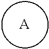

is other than optionally substituted phenyl, pyrid-2-yl or pyrid-3-yl. In certain embodiments of the present invention

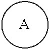

is other than optionally substituted phenyl or pyridyl. In certain embodiments of the present invention

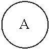

is other than optionally substituted thein-2-yl. In certain embodiments of the present invention

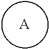

is other than optionally substituted benzothein-2-yl. In certain embodiments of the present invention

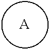

is other than optionally substituted thien-2-yl or benzothien-2-yl. In certain embodiments of the present invention

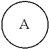

is other than optionally substituted phenyl, pyrid-2-yl, pyrid-3-yl, thien-2-yl or benzothein-2-yl.

In certain embodiments of the present invention, R⁰ is fluoro, R¹ is hydroxymethyl-, R^{1a} is hydrogen, R² is hydrogen, R³ is hydrogen, R⁴ is selected from the group consisting of hydrogen, halogen, methyl, ethyl, methoxy, ethoxy, trifluoromethyl, trifluoromethoxy, cyano and cyclopropyl, R⁵ is hydrogen, R⁶ is hydrogen and

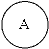

is other phenyl, pyrid-2-yl, or pyrid-3-yl; wherein the phenyl is substituted at the 4-position, wherein the pyrid-2-yl is substituted at the 4- or 5-position and wherein the pyrid-3-yl is substituted at the 5-position.

In certain embodiments of the present invention, R⁰ is fluoro, R¹ is hydroxymethyl-, R^{1a} is hydrogen, R² is hydrogen, R³ is hydrogen, R⁴ is selected from the group consisting of hydrogen, halogen, methyl, ethyl, methoxy, ethoxy, trifluoromethyl, trifluoromethoxy, cyano and cyclopropyl, R⁵ is hydrogen, R⁶ is hydrogen and

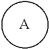

is other phenyl, pyrid-2-yl, pyrid-3-yl, thien-2-yl or benzothien-2-yl; wherein the phenyl, pyrid-2-yl, pyrid-3yl, thien-2-yl or benzothien-2-yl is optionally substituted.

In certain embodiments of the present invention

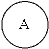

is other than phenyl, wherein the phenyl is optionally substituted with one to two substituents independently selected from the group consisting of halogen, C₁₋₂alkyl, C₁₋₂alkoxy, trifluoromethyl and trifluoromethoxy. In certain embodiments of the present invention

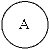

is other than phenyl, wherein the phenyl is optionally substituted with one to two substituents independently selected from the group consisting of halogen, C₁₋₂alkyl, C₁₋₂alkoxy and trifluoromethoxy.

In certain embodiments of the present invention

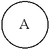

is other than 4-chloro-phenyl, 3-fluoro-4-methyl-phenyl, 3-bromo-4-methoxy-phenyl, 4-methyl-phenyl, 4-ethyl-phenyl, 3-methoxy-phenyl, 4-methoxy-phenyl, 4-ethoxy-phenyl and 4-(trifluoro-methoxy)-phenyl.

In certain embodiments of the present invention R² is other than hydrogen. In certain embodiments of the present invention, R² and R⁴ are each independently selected to be a substituent other than hydrogen.

In certain embodiment, the present invention is directed to a compound selected from the group consisting of
(2S,3R,4R,5S,6R)-2-(5-((2,3-dihydrobenzo[b][1,4]dioxin-6-yl)methyl)-2-hydroxy-4-methoxyphenyl)-5-fluoro-6-(hydroxymethyl)-5-methyl-tetrahydro-2H-pyran-3,4-diol;
(2S,3R,4R,6R)-2-(5-(benzo[b]thiophen-2-ylmethyl)-2-hydroxy-4-methylphenyl)-5,5-difluoro-6-(hydroxymethyl)tetrahydro-2H-pyran-3,4-diol;
(2S,3R,4R,6R)-2-[5-(benzothiophen-2-ylmethyl)-2-hydroxy-4-methoxy-phenyl]-5,5-difluoro-6-(hydroxymethyl)tetrahydropyran-3,4-diol;
(2S,3R,4R,6R)-2-(5-(chroman-6-ylmethyl)-2-hydroxy-4-methoxyphenyl)-5,5-difluoro-6-(hydroxymethyl)tetrahydro-2H-pyran-3,4-diol;
(2S,3R,4R,6R)-2-(5-((2,3-dihydrobenzo[b][1,4]dioxin-6-yl)methyl)-2-hydroxy-4-methylphenyl)-5,5-difluoro-6-(hydroxymethyl)tetrahydro-2H-pyran-3,4-diol;
(2S,3R,4R,6R)-2-(4-chloro-2-hydroxy-5-(4-methoxybenzyl)phenyl)-5,5-difluoro-6-(hydroxymethyl)-tetrahydro-2H-pyran-3,4-diol;
(2S,3R,4R,6R)-2-(4-ethyl-2-hydroxy-5-(4-methoxybenzyl)phenyl)-5,5-difluoro-6-(hydroxymethyl)-tetrahydro-2H-pyran-3,4-diol;
(2S,3R,4R,6R)-5,5-difluoro-2-(2-hydroxy-4-methoxy-5-(4-methoxybenzyl)phenyl)-6-(hydroxymethyl)-tetrahydro-2H-pyran-3,4-diol;
(2S,3R,4R,6R)-2-(4-chloro-5-((2,3-dihydrobenzofuran-6-yl)methyl)-2-hydroxyphenyl)-5,5-difluoro-6-(hydroxymethyl)-tetrahydro-2H-pyran-3,4-diol;

and isotopologues and pharmaceutically acceptable salts thereof.

Additional embodiments of the present invention, include those wherein the substituents selected for one or more of the variables defined herein (i.e. R¹, R², R³, R⁴, R⁵, R⁶,

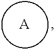

etc.) are independently selected to be any individual substituent or any subset of substituents selected from the complete list as defined herein. Additional embodiments of the present invention, include those wherein the substituents selected for one or more of the variables defined herein (i.e. R¹, R², R³, R⁴, R⁵, R⁶,

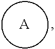

etc.) are independently selected to correspond to any of the embodiments as defined herein.

In another embodiment of the present invention is any single compound or subset of compounds selected from the representative compounds listed in Tables 1 to 2 below.

Representative compounds of the present invention are as listed in Table 1 to 2 below. Unless otherwise noted, wherein a stereogenic center is present in the listed compound, the compound was prepared as a mixture of stereo-configurations.

TABLE 1
Representative Compounds of Formula (I) wherein R⁰ is methyl

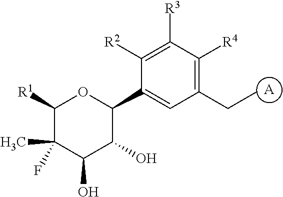

| ID No. | R¹ | R² | R³ | R⁴ | 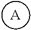 |
|---|---|---|---|---|---|
| 1 | —CH₂—OH | hydroxy | H | methyl | benzothien-2-yl |
| 2 | —CH₂—OH | hydroxy | H | methoxy | chroman-6-yl |
| 3 | —CH₂—OH | hydroxy | H | chloro | 2,3-dihydro- |
| | | | | | benzo[b][1,4] |
| | | | | | dioxin-6-yl |
| 4 | —CH₂—OH | methoxy | H | methoxy | chroman-6-yl |
| 5 | —CH₂—OH | hydroxy | H | methyl | 5-fluoro- |
| | | | | | benzothien-2-yl |
| 6 | —CH₂—OH | hydroxy | H | methoxy | 2,3-dihydro- |
| | | | | | benzo[b][1,4] |
| | | | | | dioxin-6-yl |
| 7 | —CH₂—OH | methoxy | H | methyl | 5-fluoro- |
| | | | | | benzothien-2-yl |
| 8 | —CH₂—OH | H | H | chloro | 2,3-dihydro- |
| | | | | | benzo[b][1,4] |
| | | | | | dioxin-6-yl |
| 9 | —CH₂—OH | methoxy | H | methoxy | 2,3-dihydro- |
| | | | | | benzo[b][1,4] |
| | | | | | dioxin-6-yl |
| 10 | —CH₂—OH | hydroxy | H | chloro | benzothien-2-yl |
| 11 | —CH₂—OH | methoxy | H | chloro | benzothien-2-yl |
| 14 | —CH₂—OH | H | H | methoxy | 2,3-dihydro- |
| | | | | | benzo[b][1,4] |
| | | | | | dioxin-6-yl |
| 15 | —CH₂—OH | H | H | ethyl | 2,3-dihydro- |
| | | | | | benzo[b][1,4] |
| | | | | | dioxin-6-yl |
| 16 | —CH₂—OH | hydroxy | H | ethyl | 4-methoxy-phenyl |
| 17 | —CH₂—OH | methoxy | H | ethyl | 4-methoxy-phenyl |
| 21 | —CH₂—OH | hydroxy | iodo | methoxy | 2,3-dihydro- |
| | | | | | benzo[b][1,4] |
| | | | | | dioxin-6-yl |
| 23 | —CH₂—OH | hydroxy | H | methyl | 4-(fluoro- |
| | | | | | methoxy)-phenyl |
| 24 | —CH₂—OH | methoxy | H | methyl | 4-(fluoro- |
| | | | | | methoxy)-phenyl |

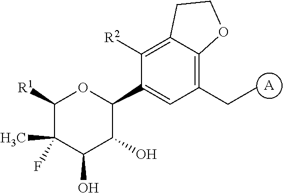

| ID No. | R¹ | R² | 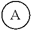 |
|---|---|---|---|
| 19 | —CH₂—OH | H | 2,3-dihydro-benzo[b][1,4]dioxin- |
| | | | 6-yl |

TABLE 2
Representative Compounds of Formula (I) where R⁰ is Fluoro.

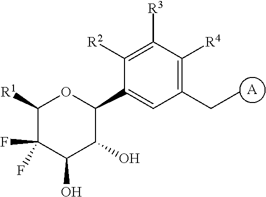

| ID No. | R¹ | R² | R³ | R⁴ | 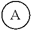 |
|---|---|---|---|---|---|
| 25 | —CH₂OH | hydroxy | H | methyl | benzothien-2-yl |
| 26 | —CH₂OH | hydroxy | H | ethyl | benzothien-2-yl |
| 27 | —CH₂OH | hydroxy | H | chloro | benzothien-2-yl |
| 31 | —CH₂OH | hydroxy | H | methoxy | benzothien-2-yl |
| 35 | —CH₂OH | methoxy | H | methyl | benzothien-2-yl |
| 36 | —CH₂OH | methoxy | H | chloro | benzothien-2-yl |
| 39 | —CH₂OH | methoxy- | H | methyl | benzothien-2-yl |
| | | ethoxy- | | | |
| 40 | —CH₂OH | cyano- | H | methyl | benzothien-2-yl |
| | | methoxy- | | | |
| 41 | —CH₂OH | ethoxy | H | methyl | benzothien-2-yl |
| 42 | —CH₂OH | isopropyl- | H | methyl | benzothien-2-yl |
| | | oxy | | | |
| 44 | —CH₂OH | hydroxy | H | methoxy | chroman-6-yl |
| 45 | —CH₂OH | hydroxy | H | chloro | 2,3-dihydro- |
| | | | | | benzo[b][1,4]dioxin- |
| | | | | | 6-yl |
| 46 | —CH₂OH | hydroxy | H | chloro | 4-((S)-tetrahydro- |
| | | | | | furan-3-yl)-phenyl) |
| 47 | —CH₂OH | hydroxy | H | methyl | 4-ethoxy-phenyl |
| 48 | —CH₂OH | methoxy | H | chloro | 2,3-dihydro- |
| | | | | | benzo[b][1,4]dioxin- |
| | | | | | 6-yl |
| 49 | —CH₂OH | methoxy | H | methyl | 4-ethoxy-phenyl |
| 50 | —CH₂OH | methoxy | H | chloro | 4-((S)-tetrahydro- |
| | | | | | furan-3-yl)-phenyl) |
| 51 | —CH₂OH | cyano- | H | chloro | benzothien-2-yl |
| | | methoxy- | | | |
| 52 | —CH₂OH | hydroxy | H | methyl | 5-fluoro- |
| | | | | | benzothien-2-yl |
| 53 | —CH₂OH | methoxy | H | methyl | 5-fluoro- |
| | | | | | benzothien-2-yl |
| 54 | —CH₂OH | hydroxy | H | methoxy | 2,3-dihydro- |
| | | | | | benzo[b][1,4]dioxin- |
| | | | | | 6-yl |
| 55 | —CH₂OH | methoxy | H | methoxy | 2,3-dihydro- |
| | | | | | benzo[b][1,4]dioxin- |
| | | | | | 6-yl |
| 56 | —CH₂OH | hydroxy | H | methyl | 2,3-dihydro- |
| | | | | | benzo[b][1,4]dioxin- |
| | | | | | 6-yl |
| 60 | —CH₂OH | hydroxy | methyl | methyl | benzothien-2-yl |
| 62 | —CH₂OH | hydroxy | H | methoxy | 2,3-dihydro- |
| | | | | | benzofuran-5-yl |
| 64 | —CH₂OH | hydroxy | H | chloro | 5-(methoxy- |
| | | | | | carbonyl)-6,7- |
| | | | | | dihydro-thieno[3,2- |
| | | | | | c]pyridin-2-yl |
| 65 | —CH₂OH | methoxy | H | chloro | 5-(methoxy- |
| | | | | | carbonyl)-6,7- |
| | | | | | dihydro-thieno[3,2- |
| | | | | | c]pyridin-2-yl |
| 68 | —CH₂OH | hydroxy | H | methyl | 5-(4-fluoro-phenyl)- |
| | | | | | thien-2-yl |
| 69 | —CH₂OH | hydroxy | H | methyl | 5-(cyclopentyl- |
| | | | | | carbonyl)-6,7- |
| | | | | | dihydro-thieno[3,2- |
| | | | | | c]pyridin-2-yl |
| 70 | —CH₂OH | hydroxy | H | methyl | 5-(6-fluoro-pyrid-2- |
| | | | | | yl)-thien-2-yl |
| 71 | —CH₂OH | hydroxy | H | methyl | 5-(3-fluoro-phenyl)- |
| | | | | | thien-2-yl |
| 72 | —CH₂OH | hydroxy | H | chloro | 5-(methyl- |
| | | | | | carbonyl)-6,7- |
| | | | | | dihydro-thieno[3,2- |
| | | | | | c]pyridin-2-yl |
| 73 | —CH₂OH | methoxy | H | chloro | 5-(methyl- |
| | | | | | carbonyl)-6,7- |
| | | | | | dihydro-thieno[3,2- |
| | | | | | c]pyridin-2-yl |
| 75 | —CH₂OH | methoxy | H | methoxy | 5-(methyoxy- |
| | | | | | carbonyl)-6,7- |
| | | | | | dihydro-thieno[3,2- |
| | | | | | c]pyridin-2-yl |
| 77 | —CH₂OH | methoxy | H | methoxy | 5-(methyl- |
| | | | | | carbonyl)-6,7- |
| | | | | | dihydro-thieno[3,2- |
| | | | | | c]pyridin-2-yl |
| 78 | —CH₂OH | methoxy | H | methyl | 5-(pyrrolidin-1-yl- |
| | | | | | carbonyl)-6,7- |
| | | | | | dihydro-thieno[3,2- |
| | | | | | c]pyridin-2-yl |
| 79 | —CH₂OH | H | H | methyl | 5-(thiazol-2-yl- |
| | | | | | carbonyl)-6,7- |
| | | | | | dihydro-thieno[3,2- |
| | | | | | c]pyridin-2-yl |
| 80 | —CH₂OH | hydroxy | H | methyl | 5-(thiazol-2-yl- |
| | | | | | carbonyl)-6,7- |
| | | | | | dihydro-thieno[3,2- |
| | | | | | c]pyridin-2-yl |
| 81 | —CH₂OH | hydroxy | H | chloro | 4-methoxy-phenyl |
| 82 | —CH₂OH | hydroxy | H | methyl | 5-cyano-6,7- |
| | | | | | dihydro-thieno[3,2- |
| | | | | | c]pyridin-2-yl |
| 83 | —CH₂OH | hydroxy | H | methyl | 5-(5-methyl-thiazol- |
| | | | | | 2-yl-carbonyl)-6,7- |
| | | | | | dihydro-thieno[3,2- |
| | | | | | c]pyridin-2-yl |
| 84 | —CH₂OH | H | H | methyl | 5-(cyclopentyl- |
| | | | | | carbonyl)-6,7- |
| | | | | | dihydro-thieno[3,2- |
| | | | | | c]pyridin-2-yl |
| 86 | —CH₂OH | hydroxy | H | H | 4-methoxy-phenyl |
| 87 | —CH₂OH | methoxy | H | chloro | 4-methoxy-phenyl |
| 88 | —CH₂OH | hydroxy | H | ethyl | 4-methoxy-phenyl |
| 89 | —CH₂OH | methoxy | H | ethyl | 4-methoxy-phenyl |
| 91 | —CH₂OH | hydroxy | H | methoxy | 4-methoxy-phenyl |
| 92 | —CH₂OH | methoxy | H | methoxy | 4-methoxy-phenyl |
| 96 | —CH₂OH | hydroxy | H | methyl | 4-(difluoro- |
| | | | | | methoxy)-phenyl |
| 99 | —CH₂OH | hydroxy | H | methyl | 4-(trifluoro- |
| | | | | | methoxy)-phenyl |
| 101 | —CH₂OH | methoxy | H | methyl | 4-(trifluoro- |
| | | | | | methoxy)-phenyl |
| 104 | —CH₂OH | hydroxy | H | ethyl | 4-(trifluoro- |
| | | | | | methoxy)-phenyl |
| 105 | —CH₂OH | hydroxy | H | chloro | 4-(trifluoro- |
| | | | | | methoxy)-phenyl |
| 106 | —CH₂OH | methoxy | H | chloro | 4-(trifluoro- |
| | | | | | methoxy)-phenyl |
| 108 | —CH₂OH | hydroxy | H | methyl | 4-methoxy-phenyl |
| 109 | —CH₂OH | methoxy | H | ethyl | 4-(trifluoro- |
| | | | | | methoxy)-phenyl |
| 110 | —CH₂OH | hydroxy | H | methyl | 5-chloro-thien-2-yl |
| 111 | —CH₂OH | hydroxy | H | chloro | 2,3-dihydro- |
| | | | | | benzofuran-6-yl |
| 112 | —CH₂OH | methoxy | H | methyl | 5-chloro-thien-2-yl |
| 113 | —CH₂OH | hydroxy | H | ethyl | 3-bromo-4- |
| | | | | | methoxy-phenyl |
| 115 | —CH₂OH | methoxy | H | chloro | 2,3-dihydro- |
| | | | | | benzofuran-6-yl |
| 116 | —CH₂OH | hydroxy | H | chloro | 4-(fluoro-methoxy)- |
| | | | | | phenyl |
| 119 | —CH₂OH | hydroxy | H | methoxy | 4-(trifluoro- |
| | | | | | methoxy)-phenyl |
| 121 | —CH₂OH | methoxy | H | methoxy | 4-(trifluoro- |
| | | | | | methoxy)-phenyl |
| 123 | —CH₂OH | methoxy | H | methyl | 4-(cyclopropyl)- |
| | | | | | phenyl |
| 124 | —CH₂OH | hydroxy | H | methyl | 4-(cyclopropyl)- |
| | | | | | phenyl |
| 125 | —CH₂OH | hydroxy | H | chloro | 4-(fluoro-methoxy)- |
| | | | | | phenyl |
| 127 | —CH₂OH | hydroxy | H | chloro | 4-ethyl-phenyl |
| 128 | —CH₂OH | hydroxy | H | ethyl | 4-ethyl-phenyl |
| 129 | —CH₂OH | methoxy | H | methyl | 4-ethyl-phenyl |
| 130 | —CH₂OH | methoxy | H | ethyl | 4-ethyl-phenyl |
| 131 | —CH₂OH | hydroxy | H | methyl | 4-ethyl-phenyl |
| 133 | —CH₂OH | hydroxy | H | methyl | 4-(fluoro-methoxy)- |
| | | | | | phenyl |
| 134 | —CH₂OH | hydroxy | H | methoxy | 5-cyano-6,7- |
| | | | | | dihydro-thieno[3,2- |
| | | | | | c]pyridin-2-yl |
| 135 | —CH₂OH | methoxy | H | chloro | 4-ethyl-phenyl |
| 136 | —CH₂OH | hydroxy | H | chloro | 4-(cyclopropyl)- |
| | | | | | phenyl |
| 137 | —CH₂OH | hydroxy | H | cyclo- | 4-ethyl-phenyl |
| | | | | propyl | |
| 138 | —CH₂OH | methoxy | H | methyl | 4-(fluoro-methoxy)- |
| | | | | | phenyl |
| 140 | —CH₂OH | hydroxy | H | methoxy | 4-(cyclopropyl)- |
| | | | | | phenyl |
| 141 | —CH₂OH | hydroxy | H | cyclo- | 4-(cyclopropyl)- |
| | | | | propyl | phenyl |
| 142 | —CH₂OH | hydroxy | H | methoxy | 4-(fluoro-methoxy)- |
| | | | | | phenyl |
| 143 | —CH₂OH | hydroxy | H | cyclo- | 4-(fluoro-methoxy)- |
| | | | | propyl | phenyl |
| 144 | —CH₂OH | methoxy | H | methoxy | 4-(cyclopropyl)- |
| | | | | | phenyl |
| 145 | —CH₂OH | hydroxy | H | methoxy | 4-ethyl-phenyl |
| 147 | —CH₂OH | methoxy | H | methoxy | 4-(fluoro-methoxy)- |
| | | | | | phenyl |
| 148 | —CH₂OH | hydroxy | H | ethyl | 4-(fluoro-methoxy)- |
| | | | | | phenyl |
| 149 | —CH₂OH | hydroxy | bromo | ethyl | 4-methoxy-phenyl |
| 150 | —CH₂OH | hydroxy | H | ethyl | 4-(cyclopropyl)- |
| | | | | | phenyl |
| 151 | —CH₂OH | methoxy | H | ethyl | 4-(cyclopropyl)- |
| | | | | | phenyl |
| 152 | —CH₂OH | hydroxy | H | cyano | 4-(dimethylamino)- |
| | | | | | phenyl |
| 153 | —CH₂OH | methoxy | H | methoxy | 4-ethyl-phenyl |
| 157 | —CH₂OH | hydroxy | H | chloro | 3-methoxy-phenyl |
| 161 | —CH₂OH | hydroxy | H | chloro | 5-methyl-thien-2-yl |
| 162 | —CH₂OH | hydroxy | H | chloro | 4-ethoxy-phenyl |
| 163 | —CH₂OH | hydroxy | H | chloro | 4-chloro-phenyl |
| 165 | —CH₂OH | methoxy | H | chloro | 5-methyl-thien-2-yl |
| 166 | —CH₂OH | methoxy | H | cyano | 4-ethyl-phenyl |
| 167 | —CH₂OH | hydroxy | H | chloro | 5-chloro-thien-2-yl |
| 168 | —CH₂OH | methoxy | H | chloro | 4-chloro-phenyl |
| 169 | —CH₂OH | methoxy | H | cyano | 4-(cyclopropyl)- |
| | | | | | phenyl |
| 171 | —CH₂OH | hydroxy | H | chloro | 4-methyl-phenyl |
| 172 | —CH₂OH | methoxy | H | chloro | 5-chloro-thien-2-yl |
| 173 | —CH₂OH | hydroxy | H | cyano | 4-(cyclopropyl)- |
| | | | | | phenyl |
| 175 | —CH₂OH | hydroxy | H | cyano | 4-ethyl-phenyl |
| 176 | —CH₂OH | methoxy | H | chloro | 4-methyl-phenyl |
| 177 | —CH₂OH | hydroxy | H | methyl | 3-fluoro-4-methyl- |
| | | | | | phenyl |
| 181 | —CH₂OH | methoxy | H | methyl | 4-methyl-2H- |
| | | | | | benzo[b][1,4]oxazin- |
| | | | | | 7-yl-3-one |
| 182 | —CH₂OH | hydroxy | H | methyl | 4-ethyl-thien-2-yl |

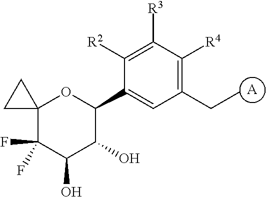

| ID No. | R² | R³ | R⁴ | 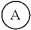 |
|---|---|---|---|---|
| 74 | H | H | methyl | 4-ethoxy-phenyl |
| 85 | H | H | ethyl | 2,3-dihydro- |
| | | | | benzo[b][1,4]dioxin-6-yl |
| 94 | hydroxy | H | ethyl | 2,3-dihydro- |
| | | | | benzo[b][1,4]dioxin-6-yl |

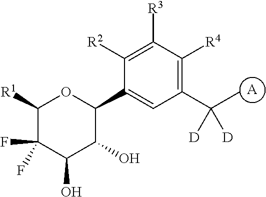

| ID No. | R¹ | R² | R³ | R⁴ | 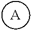 |
|---|---|---|---|---|---|
| 192 | —CH₂OH | H | H | ethyl | 2,3-dihydro- |
| | | | | | benzo[b][1,4]dioxin-6-yl |

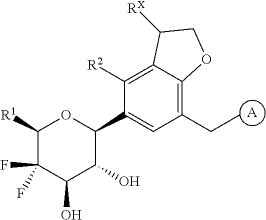

| ID No. | R¹ | R² | R^{x} | 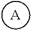 |
|---|---|---|---|---|
| 30 | —CH₂OH | hydroxy | H | 4-ethyl-phenyl |
| 38 | —CH₂OH | methoxy | H | 4-ethyl-pehnyl |
| 58 | —CH₂OH | H | H | 4-ethyl-phenyl |
| 61 | —CH₂OH | H | —CH₂OH | 4-ethyl-phenyl |
| 76 | —CH₂OH | methoxy | H | 2,3-dihydrobenzo[b][1,4] |
| | | | | dioxin-6-yl |
| 146 | —CH₂OH | H | H | 4-(fluoro-methoxy)-phenyl |
| 174 | —CH₂OH | H | H | 4-(cyclopropyl)-phenyl |
| 179 | —CH₂OH | H | H | 4-(dimethylamino)-phenyl |
| 183 | —CH₂OH | methoxy | H | 4-(cyclopropyl)-phenyl |
| 184 | —CH₂OH | methoxy | H | 4-(fluoro-methoxy)-phenyl |
| 185 | —CH₂OH | methoxy | H | 4-(dimethylamino)-phenyl |

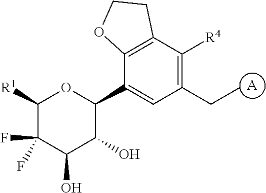

| ID No. | R¹ | R⁴ | 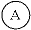 |
|---|---|---|---|
| 93 | —CH₂OH | methoxy | chroman-6-yl |
| 95 | —CH₂OH | methoxy | 2,3-dihydrobenzo[b][1,4]oxathiin-6-yl |
| 103 | —CH₂OH | methyl | chroman-6-yl |
| 114 | —CH₂OH | chloro | 2,3-dihydro-benzofuran-5-yl |
| 118 | —CH₂OH | methyl | 2,3-dihydrobenzo[b][1,4]oxathiin-6-yl- |
| | | | 4,4-dioxide |
| 126 | —CH₂OH | chloro | chroman-6-yl |
| 132 | —CH₂OH | chloro | 2,3-dihydrobenzo[b][1,4]oxathiin-6-yl |
| 139 | —CH₂OH | methyl | 2,3-dihydrobenzo[b][1,4]oxathiin-6-yl |
| 189 | —CH₂OH | methyl | benzothien-2-yl |

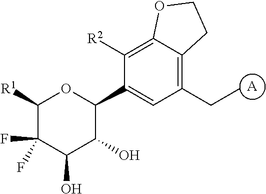

| ID No | R¹ | R² | 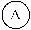 |
|---|---|---|---|
| 187 | —CH₂OH | hydroxy | 2,3-dihydrobenzo[b][1,4]dioxin-6-yl |

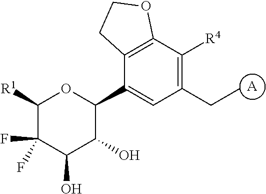

| ID No | R¹ | R⁴ | 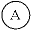 |
|---|---|---|---|
| 188 | —CH₂OH | chloro | 2,3-dihydrobenzo[b][1,4]dioxin-6-yl |

### Definitions

As used herein, “halogen” shall mean chloro, bromo, fluoro and iodo.

As used herein, the term “C_{X-Y}alkyl” wherein X and Y are integers, whether used alone or as part of a substituent group, include straight and branched chains containing between X and Y carbon atoms. For example, C₁₋₄ alkyl radicals include straight and branched chains of between 1 and 4 carbon atoms, including methyl, ethyl, propyl, isopropyl, butyl, isobutyl, sec-butyl and t-butyl.

One skilled in the art will recognize that the terms “—(C_{X-Y}alkyl)- and —C_{X-Y}alkyl-” wherein X and Y are integers, shall denote any C_{X-Y}alkyl carbon chain as herein defined, wherein said C_{X-Y}alkyl chain is divalent and is further bound through two points of attachment, preferably through two terminal carbon atoms.

As used herein, unless otherwise noted, the terms “fluorinated C_{X-Y}alkyl” and “fluoro substituted C_{X-Y}alkyl” shall mean any C_{X-Y}alkyl group as defined above substituted with at least one fluorine atom, preferably one to three fluorine atoms. In an example, “fluorinated C₁₋₄alkyl” include, but are not limited, to —CH₂F, —CF₂H, —CF₃, —CH₂—CF₃, —CF₂—CF₂—CF₂—CF₃, and the like.

As used herein, unless otherwise noted, the term “hydroxy substituted C_{X-Y}alkyl” shall mean any C_{X-Y}alkyl group as defined above substituted with at least one hydroxy, preferably one to two hydroxy groups, wherein the hydroxy group(s) may be bound to any carbon atom of the C_{X-Y}alkyl, preferably, the hydroxy group(s) are bound to the terminal carbon atom. In an example, “hydroxy substituted C₁₋₄alkyl” include, but are not limited, to —CH₂OH, —CH₂—CH₂OH, —CH(OH)—CH₃, —CH(OH)—CH₂OH, —CH₂—CH₂—CH₂OH, —CH(OH)—CH₂—CH₃, —CH(OH)(CH₃)₂, and the like.

As used herein, unless otherwise noted, “C_{X-Y}alkenyl” shall denote mean any straight or branched chain of between X and Y carbon atoms, wherein the straight or branched chain contains as least one, preferably one, unsaturated double bond. For example, the term “C₂₋₆alkenyl” includes straight and branched chains of between 2 and 6 carbon atoms containing at least one, preferably one, unsaturated double bond such as vinyl, n-propen-1-yl, n-buten-1-yl, n-but-2-en-1-yl, n-but-1-en-2-yl, penten-1-yl, pent-2-en-1-yl, and the like.

As used herein, unless otherwise noted, “C₁₋₄alkoxy” shall denote an oxygen ether radical of the above described straight or branched chain alkyl groups containing one to four carbon atoms. For example, methoxy, ethoxy, n-propoxy, isopropoxy, sec-butoxy, t-butoxy, and the like.

As used herein, unless otherwise noted, the terms “fluorinated C_{X-Y}alkoxy” and “fluoro substituted C_{X-Y}alkoxy”, shall mean any C_{X-Y}alkoxy group as defined above substituted with at least one fluorine atom, preferably one to three fluorine atoms. In an example, “fluorinated C₁₋₄alkoxy” include, but are not limited, —OCH₂F, —OCF₂H, —OCF₃, —OCH₂—CF₃, —OCF₂—CF₂—CF₂—CF₃, and the like.

As used herein, unless otherwise noted, the term “cyano substituted C_{X-Y}alkoxy” shall mean any C_{X-Y}alkoxy group as defined above substituted with one cyano group, wherein the cyano group may be bound to any carbon atom of the C_{X-Y}alkoxy, preferably, the cyano group are bound to the terminal carbon atom. In an example, “cyano substituted C₁₋₄alkoxy” include, but are not limited, to —O—CH₂CN, —O—CH₂—CH₂CN, —O—CH(CN)—CH₃, —O—CH(CN)—CH₂OH, —O—CH₂—CH₂—CH₂CN, —O—CH(CN)—CH₂—CH₃, —O—CH(CN)(CH₃)₂, and the like.

As used herein, unless otherwise noted, the term “C_{X-Y}cycloalkyl”, wherein X and Y are integers, shall mean any stable X- to Y-membered monocyclic, bicyclic, polycyclic or bridges saturated ring system, preferably a monocyclic or bicyclic saturated ring system. For example, the term “C₃₋₁₂cycloalkyl” includes, but is not limited to cyclopropyl, cyclobutyl, cyclopentyl, cyclohexyl, cycloheptyl, cyclooctyl, cyclononyl, cyclodecyl, and the like.

As used herein, unless otherwise noted, the term “C_{X-Y}cycloalkenyl”, wherein X and Y are integers, shall mean any stable X- to Y-membered monocyclic, bicyclic, polycyclic or bridged, preferably a monocyclic or bicyclic, ring system containing at least one, preferably one to three, unsaturated double bonds. For example, the term “C₆₋₁₀cycloalkenyl” includes, but is not limited to cyclohexenyl, bicyclo[4.2.0]octa-1(6),2,4-trien-3-yl, and the like.

As used herein, the term “cycloprop-1,1-diyl” shall mean a substituent group of the following structure

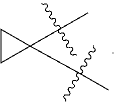 [SMILES: *C1(*)CC1]

As used herein, unless otherwise noted, “heteroaryl” shall denote any five or six membered monocyclic aromatic ring structure containing at least one heteroatom selected from the group consisting of O, N and S, optionally containing one to three additional heteroatoms independently selected from the group consisting of O, N and S; or a nine or ten membered bicyclic aromatic ring structure containing at least one heteroatom selected from the group consisting of O, N and S, optionally containing one to four additional heteroatoms independently selected from the group consisting of O, N and S. The heteroaryl group may be attached at any heteroatom or carbon atom of the ring such that the result is a stable structure.

Examples of suitable heteroaryl groups include, but are not limited to, pyrrolyl, furyl, thienyl, oxazolyl, imidazolyl, purazolyl, isoxazolyl, isothiazolyl, triazolyl, thiadiazolyl, pyridyl, pyridazinyl, pyrimidinyl, pyrazinyl, pyranyl, furazanyl, indolizinyl, indolyl, isoindolinyl, indazolyl, benzofuryl, benzothienyl, benzimidazolyl, benzthiazolyl, purinyl, quinolizinyl, quinolinyl, isoquinolinyl, isothiazolyl, cinnolinyl, phthalazinyl, quinazolinyl, quinoxalinyl, naphthyridinyl, pteridinyl, and the like.

As used herein, the term “heterocyclyl” shall denote any five to seven membered monocyclic, saturated or partially unsaturated ring structure containing at least one heteroatom selected from the group consisting of O, N and S, optionally containing one to three additional heteroatoms independently selected from the group consisting of O, N and S; or an eight to ten membered saturated, partially unsaturated, partially aromatic or benzo-fused bicyclic ring system containing at least one heteroatom selected from the group consisting of O, N and S, optionally containing one to four additional heteroatoms independently selected from the group consisting of O, N and S. The heterocyclyl group may be attached at any heteroatom or carbon atom of the ring such that the result is a stable structure.

Examples of suitable heterocyclyl groups include, but are not limited to, pyrrolinyl, pyrrolidinyl, dioxalanyl, imidazolinyl, imidazolidinyl, pyrazolinyl, pyrazolidinyl, piperidinyl, dioxanyl, morpholinyl, dithianyl, thiomorpholinyl, piperazinyl, trithianyl, indolinyl, chromenyl, 3,4-methylenedioxyphenyl, 2,3-dihydrobenzofuryl, and the like.

When a particular group is “substituted” (e.g. C_{X-Y}alkyl, C_{X-Y}alkoxy, C_{X-Y}cycloalkyl, etc.), that group may have one or more substituents, preferably from one to five substituents, more preferably from one to three substituents, most preferably from one to two substituents, independently selected from the list of substituents.

With reference to substituents, the term “independently” means that when more than one of such substituents is possible, such substituents may be the same or different from each other.

As used herein, the notation “*” shall denote the presence of a stereogenic center.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Preferably, wherein the compound is present as an enantiomer, the enantiomer is present at an enantiomeric excess of greater than or equal to about 80%, more preferably, at an enantiomeric excess of greater than or equal to about 90%, more preferably still, at an enantiomeric excess of greater than or equal to about 95%, more preferably still, at an enantiomeric excess of greater than or equal to about 98%, most preferably, at an enantiomeric excess of greater than or equal to about 99%. Similarly, wherein the compound is present as a diastereomer, the diastereomer is present at an diastereomeric excess of greater than or equal to about 80%, more preferably, at an diastereomeric excess of greater than or equal to about 90%, more preferably still, at an diastereomeric excess of greater than or equal to about 95%, more preferably still, at an diastereomeric excess of greater than or equal to about 98%, most preferably, at an diastereomeric excess of greater than or equal to about 99%.

Furthermore, some of the crystalline forms for the compounds of the present invention may exist as polymorphs and as such are intended to be included in the present invention. In addition, some of the compounds of the present invention may form solvates with water (i.e., hydrates) or common organic solvents, and such solvates are also intended to be encompassed within the scope of this invention.

As used herein, unless otherwise noted, the term “isotopologues” shall mean molecules that differ only in their isotopic composition. More particularly, an isotopologue of a molecule differs from the parent molecule in that it contains at least one atom which is an isotope (i.e. has a different number of neutrons from its parent atom).

For example, isotopologues of water include, but are not limited to, “light water” (HOH or H₂O), “semi-heavy water” with the deuterium isotope in equal proportion to protium (HDO or ¹H²HO), “heavy water” with two deuterium isotopes of hydrogen per molecule (D₂O or ²H₂O), “super-heavy water” or tritiated water (T₂O or ³H₂O), where the hydrogen atoms are replaced with tritium (³H) isotopes, two heavy-oxygen water isotopologues (H₂¹⁸O and H₂¹⁷O) and isotopologues where the hydrogen and oxygen atoms may each independently be replaced by isotopes, for example the doubly labeled water isotopologue D₂¹⁸.

It is intended that within the scope of the present invention, any one or more element(s), in particular when mentioned in relation to a compound of formula (I), shall comprise all isotopes and isotopic mixtures of said element(s), either naturally occurring or synthetically produced, either with natural abundance or in an isotopically enriched form. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O. The isotopes may be radioactive or non-radioactive. Radiolabelled compounds of formula (I) may comprise one or more radioactive isotope(s) selected from the group of ³H, ¹¹C, ¹⁸F, ¹²²I, ¹²³I, ¹²⁵I, ¹³¹I, ⁷⁵Br, ⁷⁶Br, ⁷⁷Br and ⁸²Br. Preferably, the radioactive isotope is selected from the group of ³H, ¹¹C and ¹⁸F.

In certain embodiments, the present invention is directed to compounds of formula (I) wherein R⁵ and R⁶ are the same and are each deuterium.

Under standard nomenclature used throughout this disclosure, the terminal portion of the designated side chain is described first, followed by the adjacent functionality toward the point of attachment. Thus, for example, a “phenylC₁-C₆alkylaminocarbonylC₁-C₆alkyl” substituent refers to a group of the formula

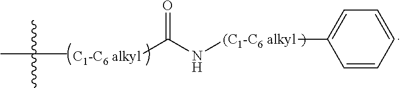

One skilled in the art will recognize that when R² and R³ or R³ and R⁴ are taken together with the carbon atoms to which they are bound to form 2,3-dihydrofuranyl, said 2,3-dihydrofuranyl ring structure is one ring of a bicyclic ring structure. More particularly, when R² and R³ or R³ and R⁴ are taken together with the phenyl ring to which they are bound (through the carbon atoms to which they are bound), the resulting structure is a partially unsaturated, benzo-fused bicyclic ring structure. Thus, for example, when R² and R³ are taken together with the carbon atoms to which they are bound to form 2,3-dihydrofuranyl—a group of the following structure, with numbering order as indicated

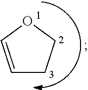

then the resulting bicyclic structure (where R² and R³ are taken together with the phenyl to which they are bound, through the carbon atoms to which they are bound) is 2,3-dihydrobenzofuranyl, a group of the following structure, with numbering order as indicated:

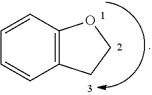

One skilled in the art will further recognize that when R² and R³ or R³ and R⁴ are taken together with the phenyl ring to which they are bound (through the carbon atoms to which they are bound) to form the corresponding benzo-fused bicyclic ring structure, said benzo-fused ring structure may exist as either of two orientations.

For example, when R² and R³ are taken together with the phenyl ring to which they are bound (through the carbon atoms to which they are bound) to form the corresponding 2,3-dihydrobenzofuranyl, then the 2,3-dihydrobenzofuranyl may be incorporated into the compound of formula (I) in either of two orientations, more particularly as the corresponding structure (R1)

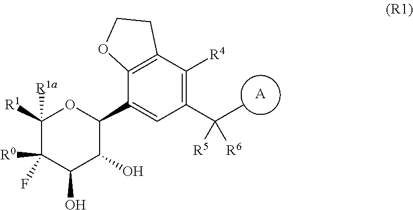

or the corresponding structure (R2)

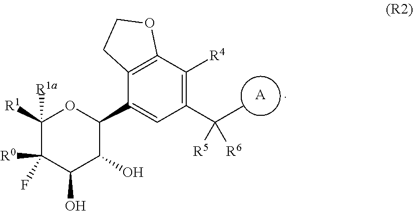

One skilled in the art will further recognize that the orientation of the compound of formula (I) can be identified by its drawn structure (for example as shown at the head of the Examples which follow hereinafter), or by the chemical name which identifies the binding orientation of the 2,3-dihydrobenzofuranyl ring structure within the complete compound of formula (I).

Abbreviations used in the specification, particularly the Schemes and Examples, are as follows:

| | | |
|---|---|---|
| 18-Crown-6 | = | 1,4,7,10,13,16-hexaoxacyclooctadecane |
| AcOH or HOAc | = | Acetic acid |
| AcOEt or EA or EtOAc | = | Ethyl Acetate |
| ACN or MeCN | = | Acetonitrile |
| ARB | = | Angiotensin receptor blockers |
| AMG | = | Alpha-Methyl Glucopyranoside |
| aq. | = | aqueous |
| AUC | = | Area Under the Cruve |
| BAST or | = | Bis(2-methoxyethyl)aminosulfur trifluoride |
| DEXOX-FLUOR ® | | |
| BF₃•Et₂O | = | Boron trifluoride diethyl etherate |
| BG | = | Blood Glucose |
| Bn or Bz | = | Benzyl |
| BnBr | = | Benzylbromide |
| Boc | = | tert-Butoxycarbonyl |
| Bu₄NCl | = | Tetrabutylammonium chloride |
| n-Bu₄NF | = | Tetra-n-butylammonium fluoride |
| n-BuLi | = | n-Butyl lithium |
| sec-BuLi | = | sec-Butyl lithium |
| t-BuLi | = | tert-Butyl lithium |
| n-Bu₄NF | = | Tetra-n-butylammonium fluoride |
| n-Bu₄NI | = | Tetra-n-butylammonium iodide |
| t-BuOH | = | tert-Butanol |
| t-BuOK | = | Potassium tert-Butoxide |
| t-BuONa or NaOt-Bu | = | Sodium tert-butoxide |
| n-BuSnH | = | n-Butyl tin hydride |
| CSA | = | Camphorsulfonic Acid |
| DAST | = | Diethylaminosulfur trifluoride |
| DBU | = | 1,8-Diazabicyclo[5.4.0]undec-7-ene |
| DCM | = | Dichloromethane |
| DDQ | = | 2,3-Dichloro-5,6-dicyano-1,4-benzoquinone |
| Dess-Martin | = | 1,1,1-Triacetoxy-1,1-dihydro-1,2-benziodoxol- |
| Periodinane or Martin's | | 3(1H)-one |
| Reagent | | |
| DIBAL or DIBAL-H | = | Diisobutylaluminium hydride |
| DIPEA or DIEA | = | Diisopropylethylamine |
| DMF | = | N,N-Dimethylformamide |
| DMSO | = | Dimethylsulfoxide |
| EDCI | = | 1-Ethyl-3-(3-dimetylaminopropyl)carbodiimide |
| Et₂O | = | Diethyl Ether |
| Et₃N or TEA | = | Triethylamine |
| Et₃SiH | = | Triethylsilane |
| EtOAc or EA or AcOEt | = | Ethyl acetate |
| EtOH | = | Ethanol |
| GCMS | = | Gas chrmatography-mass spectrometry |
| HATU | = | O-(7-Azabenzotriazol-1-yl)-N,N,N″,N″-Tetramethyl |
| | | Uronium Hexafluorophosphate |
| HDL | = | High density lipoprotein |
| HEPES | = | 4-(2-Hydroxyethyl)-1-Piperizine EthaneSulfonic |
| | | Acid |
| HOBT or HOBt | = | 1-Hydroxybenzotriazole |
| HMPA | = | Hexamethylphosphoramide |
| HPLC | = | High Pressure Liquid Chromatography |
| IDDM | = | Insulin-dependent Diabetes Mellitus |
| IFG | = | Impaired fasting glucose |
| IGT | = | Impaired glucose tolerance |
| IRS | = | Insulin resistance syndrome |
| LAH or LiAlH₄ | = | Lithium aluminum hydride |
| LC-MS or LCMS | = | Liquid chromatography-masss spectrometry |
| LDL | = | Low Density Lipoprotein |
| Me | = | Methyl (i.e. —CH₃) |
| MeCN or ACN | = | Acetonitrile |
| MeOH | = | Methanol |
| 2-Me-THF or | = | 2-Methyl-tetrahydrofuran |
| 2-methyl-THF | | |
| Me₃SiH or TMS | = | Trimethylsilane |
| mesylate | = | Methanesulfonate (i.e. —O—SO₂—CH₃) |
| MOM | = | Methoxy methyl |
| MS (Å) | = | Molecular Sieves (Angstroms) |
| MTBE | = | Methyl t-butyl ether |
| NAFLD | = | Non-alcoholic fatty liver disease |
| NASH | = | Non-alcoholic steatohepatitis, |
| NBS | = | N-Bromosuccinimide |
| NIS | = | N-Iodosuccinimide |
| NIDDM | = | Non-insulin-dependent Diabetes Mellitus |
| NMO | = | N-Methylmorpholine-N-oxide |
| NMR | = | Nuclear magnetic resonance |
| —OBn or BnO— | = | —O-benzyl |
| OGTT | = | Oral Glucose Tolerance Test |
| —O—PMBor PMBO— | = | —O-(p-methoxybenzyl) |
| —OTMS or TMSO— | = | —O-(trimethylsilyl) |
| OXONE ® | = | Potassium peroxymonosulfate |
| Pb(OAc)₄ | = | Lead (IV) acetate |
| PCC | = | Pyridinium chlorochromate |
| Pd/C | = | Palladium on Carbon (catalyst) |
| Pd(PPh₃)₄ | = | Tetrakistriphenylphosphine palladium (0) |
| PE | = | Petroleum ether |
| PhI(OAc)₂ | = | (Diacetoxyiodo)benzene |
| PPh₃ | = | Triphenylphosphine |
| PMB | = | p-Methoxybenzyl |
| PMB-Cl | = | p-Methoxybenzyl chloride |
| PPTS | = | Pyridinium p-toluenesulfonate |
| iPrMg•LiCl | = | Isopropyl magnesium chloride/Lithium chloride |
| | | complex |
| sat. | = | saturated |
| SGLT | = | Sodium glucose transport |
| SGLT1 | = | Sodium glucose transport-1 |
| SGLT2 | = | Sodium glucose transport-2 |
| TBDMS | = | tert-Butyldimethylsilyl |
| TBDPS | = | tert-Butyl-diphenyl-silyl |
| TEA | = | Triethylamine |
| TES | = | Triethylsilyl |
| TFA | = | Trifluoroacetic Acid |
| THF | = | Tetrahydrofuran |
| THP | = | Tetrahydropyran |
| TLC | = | Thin Layer Chromatography |
| TMS | = | Trimethylsilyl |
| TMEDA | = | Tetramethylethylenediamine |
| TMSOTf | = | Trimethylsilyl trifluoromethanesulfonate |
| Tosylate | = | p-Toluenesulfonate (i.e. —O—SO₂-(p-methylphenyl)) |
| Tris HCl or Tris-Cl | = | Tris[hydroxymethyl]aminomethyl hydrochloride |
| or Tris base | | |
| UGE | = | Urine Glucose Excretion |

As used herein, unless otherwise noted, the term “isolated form” shall mean that the compound is present in a form which is separate from any solid mixture with another compound(s), solvent system or biological environment. In an embodiment of the present invention, the compound of formula (I) is present in an isolated form.

As used herein, unless otherwise noted, the term “substantially pure form” shall mean that the mole percent of impurities in the isolated compound is less than about 5 mole percent, preferably less than about 2 mole percent, more preferably, less than about 0.5 mole percent, most preferably, less than about 0.1 mole percent. In an embodiment of the present invention, the compound of formula (I) is present as a substantially pure form.

As used herein, unless otherwise noted, the term “substantially free of a corresponding salt form(s)” when used to described the compound of formula (I) shall mean that mole percent of the corresponding salt form(s) in the isolated base of formula (I) is less than about 5 mole percent, preferably less than about 2 mole percent, more preferably, less than about 0.5 mole percent, most preferably less than about 0.1 mole percent. In an embodiment of the present invention, the compound of formula (I) is present in a form which is substantially free of corresponding salt form(s).

As used herein, unless otherwise noted, the terms “treating”, “treatment” and the like, shall include the management and care of a subject or patient (preferably mammal, more preferably human) for the purpose of combating a disease, condition, or disorder and includes the administration of a compound of the present invention to prevent the onset of the symptoms or complications, alleviate the symptoms or complications, slow the progression of the disease or disorder, or eliminate the disease, condition, or disorder.

As used herein, unless otherwise noted, the term “prevention” shall include (a) reduction in the frequency of one or more symptoms; (b) reduction in the severity of one or more symptoms; (c) the delay or avoidance of the development of additional symptoms; and/or (d) delay or avoidance of the development of the disorder or condition.

One skilled in the art will recognize that wherein the present invention is directed to methods of prevention, a subject in need of thereof (i.e. a subject in need of prevention) shall include any subject or patient (preferably a mammal, more preferably a human) who has experienced or exhibited at least one symptom of the disorder, disease or condition to be prevented. Further, a subject in need thereof may additionally be a subject (preferably a mammal, more preferably a human) who has not exhibited any symptoms of the disorder, disease or condition to be prevented, but who has been deemed by a physician, clinician or other medical profession to be at risk of developing said disorder, disease or condition. For example, the subject may be deemed at risk of developing a disorder, disease or condition (and therefore in need of prevention or preventive treatment) as a consequence of the subject's medical history, including, but not limited to, family history, pre-disposition, co-existing (comorbid) disorders or conditions, genetic testing, and the like.

The term “subject” as used herein, refers to an animal, preferably a mammal, most preferably a human, who has been the object of treatment, observation or experiment. Preferably, the subject has experienced and/or exhibited at least one symptom of the disease or disorder to be treated and/or prevented.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

As used herein, the term “composition” is intended to encompass a product comprising the specified ingredients in the specified amounts, as well as any product which results, directly or indirectly, from combinations of the specified ingredients in the specified amounts.

As more extensively provided in this written description, terms such as “reacting” and “reacted” are used herein in reference to a chemical entity that is any one of: (a) the actually recited form of such chemical entity, and (b) any of the forms of such chemical entity in the medium in which the compound is being considered when named.

One skilled in the art will recognize that, where not otherwise specified, the reaction step(s) is performed under suitable conditions, according to known methods, to provide the desired product. One skilled in the art will further recognize that, in the specification and claims as presented herein, wherein a reagent or reagent class/type (e.g. base, solvent, etc.) is recited in more than one step of a process, the individual reagents are independently selected for each reaction step and may be the same of different from each other. For example wherein two steps of a process recite an organic or inorganic base as a reagent, the organic or inorganic base selected for the first step may be the same or different than the organic or inorganic base of the second step. Further, one skilled in the art will recognize that wherein a reaction step of the present invention may be carried out in a variety of solvents or solvent systems, said reaction step may also be carried out in a mixture of the suitable solvents or solvent systems.

One skilled in the art will recognize that wherein a reaction step of the present invention may be carried out in a variety of solvents or solvent systems, said reaction step may also be carried out in a mixture of the suitable solvents or solvent systems.

One skilled in the art will further recognize that the reaction or process step(s) as herein described are allowed to proceed for a sufficient period of time until the reaction is complete, as determined by any method known to one skilled in the art, for example, chromatography (e.g. HPLC). In this context a “completed reaction or process step” shall mean that the reaction mixture contains a significantly diminished amount of the starting material(s)/reagent(s) and a significantly reduced amount of the desired product(s), as compared to the amounts of each present at the beginning of the reaction.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

To provide a more concise description, some of the quantitative expressions herein are recited as a range from about amount X to about amount Y. It is understood that wherein a range is recited, the range is not limited to the recited upper and lower bounds, but rather includes the full range from about amount X through about amount Y, or any amount or range therein.

Examples of suitable solvents, bases, reaction temperatures, and other reaction parameters and components are provided in the detailed descriptions which follow herein. One skilled in the art will recognize that the listing of said examples is not intended, and should not be construed, as limiting in any way the invention set forth in the claims which follow thereafter.

As used herein, unless otherwise noted, the term “aprotic solvent” shall mean any solvent that does not yield a proton. Suitable examples include, but are not limited to DMF, 1,4-dioxane, THF, acetonitrile, pyridine, dichloroethane, dichloromethane, MTBE, toluene, acetone, and the like.

As used herein, unless otherwise noted, the term “leaving group” shall mean a charged or uncharged atom or group which departs during a substitution or displacement reaction. Suitable examples include, but are not limited to, Br, Cl, I, mesylate, tosylate, and the like.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

As used herein, unless otherwise noted, the term “nitrogen protecting group” shall mean a group which may be attached to a nitrogen atom to protect said nitrogen atom from participating in a reaction and which may be readily removed following the reaction. Suitable nitrogen protecting groups include, but are not limited to carbamates—groups of the formula —C(O)O—R wherein R is for example methyl, ethyl, t-butyl, benzyl, phenylethyl, CH₂═CH—CH₂—, and the like; amides—groups of the formula —C(O)—R′ wherein R′ is for example methyl, phenyl, trifluoromethyl, and the like; N-sulfonyl derivatives—groups of the formula —SO₂—R″ wherein R″ is for example tolyl, phenyl, trifluoromethyl, 2,2,5,7,8-pentamethylchroman-6-yl-, 2,3,6-trimethyl-4-methoxybenzene, and the like. Other suitable nitrogen protecting groups may be found in texts such as T. W. Greene & P. G. M. Wuts, Protective Groups in Organic Synthesis, John Wiley & Sons, 1991.

As used herein, unless otherwise noted, the term “oxygen protecting group” shall mean a group which may be attached to an oxygen atom to protect said oxygen atom from participating in a reaction and which may be readily removed following the reaction. Suitable oxygen protecting groups include, but are not limited to, acetyl, benzoyl, t-butyl-dimethylsilyl, trimethylsilyl (TMS), MOM, THP, and the like. Other suitable oxygen protecting groups may be found in texts such as T. W. Greene & P. G. M. Wuts, Protective Groups in Organic Synthesis, John Wiley & Sons, 1991.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

Additionally, chiral HPLC against a standard may be used to determine percent enantiomeric excess (% ee). The enantiomeric excess may be calculated as follows

[(Rmoles−Smoles)/(Rmoles+Smoles)]×100%

where Rmoles and Smoles are the R and S mole fractions in the mixture such that Rmoles+Smoles=1. The enantiomeric excess may alternatively be calculated from the specific rotations of the desired enantiomer and the prepared mixture as follows:

ee=([σ-obs]/[α-max])×100.

The present invention includes within its scope prodrugs of the compounds of this invention. In general, such prodrugs will be functional derivatives of the compounds which are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985.

For use in medicine, the salts of the compounds of this invention refer to non-toxic “pharmaceutically acceptable salts.” Other salts may, however, be useful in the preparation of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. Thus, representative pharmaceutically acceptable salts include, but are not limited to, the following: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

Representative acids which may be used in the preparation of pharmaceutically acceptable salts include, but are not limited to, the following: acids including acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, α-oxo-glutaric acid, glycolic acid, hipuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinc acid, nitric acid, oleic acid, orotic acid, oxalic acid, palmitic acid, pamoic acid, phosphoric acid, L-pyroglutamic acid, salicylic acid, 4-amino-salicylic acid, sebaic acid, stearic acid, succinic acid, sulfuric acid, tannic acid, (+)-L-tartaric acid, thiocyanic acid, p-toluenesulfonic acid and undecylenic acid.

Representative bases which may be used in the preparation of pharmaceutically acceptable salts include, but are not limited to, the following: bases including ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

Compounds of formula (I) of the present invention may be synthesized according to the general synthesis schemes described below. The preparation of the various starting materials used in the synthesis schemes which follow hereinafter is well within the skill of persons versed in the art.

### General Synthesis Schemes:

Compounds of formula (I) wherein R¹ is hydroxy-methyl- (i.e. —CH₂OH) may be prepared as described in Scheme 1, below.

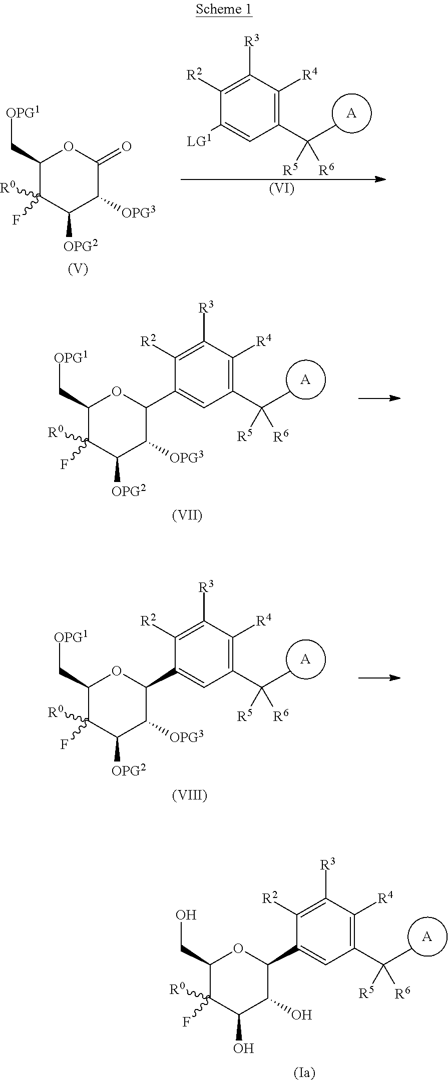

Accordingly, a suitably substituted compound of formula (V) wherein PG¹, PG² and PG³ are each an independently selected oxygen protecting group such as benzyl, trimethylsilyl (TMS), t-butyl-dimethyl-silyl (TBDMS), t-butyl-diphenyl-silyl (TBDPS), and the like, is reacted with a suitably substituted and optionally protected compound of formula (VI), wherein LG¹ is a suitably selected leaving group such as Br, Cl, I, mesylate, tosylate, trifluoromethanesulfonyl, and the like, wherein the compound of formula (VI) is pre-treated with a suitably selected organolithium or Grignard reagent such as n-BuLi, sec-BuLi, i-PrMgCl.LiCl, and the like (to yield the corresponding organometallic salt); in a suitably selected organic solvent such as THF, TMEDA (tetramethylethylenediamine), HMPA (hexamethylphosphoramide), and the like; at a temperature less than about room temperature, preferably at about −78° C.; to yield the corresponding compound of formula (VII).

The compound of formula (VII) is reacted with a suitably selected reducing agent such as a mixture of Et₃SiH and BF₃.Et₂O, Et₃SiH and TFA, and the like; in a suitably selected organic solvent such as DCM, a mixture of DCM and acetonitrile, CHCl₃, and the like; to yield the corresponding compound of formula (VIII).

The compound of formula (VIII) is de-protected (in one or more steps), according to known methods, to remove the PG¹, PG² and PG³ groups, and if present, any protecting groups introduced through the compound of formula (VI); to yield the corresponding compound of formula (Ia).

For example, wherein one or more of PG¹, PG² and/or PG³ are benzyl, said group(s) may be removed by reacting with a BCl₃ in the presence of 1,2,3,4,5-pentamethylbenzene, in an organic solvent such as DCM, at about −78° C.; or by reacting with hydrogen in the presence of Pd/C, in an organic solvent such as MeOH; or reacting with hydrogen, in the presence of Pd(OH)₂/C, in a mixture of organic solvents such as ethyl acetate and methanol. In another example, wherein one or more of PG¹, PG² and/or PG³ are a suitably selected silyl group such as trimethylsilyl (TMS), t-butyl-dimethyl-silyl (TBDMS), t-butyl-diphenyl-silyl (TBDPS), and the like, said group(s) may be removed by reacting with a suitably selected reagent or mixture of reagents such as n-Bu₄NF, HF-pyridine, KF, Bu₄NCl/KF.H₂O, and the like; wherein the reaction with HF, NH₄F—HF, Bu₄NCl/KF.H₂O, and the like is in a suitably selected organic solvent such as THF, 18-crown-6, methanol, acetonitrile, cyclohexane, and the like.

Compounds of formula (I) wherein R¹ is hydroxy-methyl- (i.e. —CH₂—OH) and wherein R⁵ and R⁶ are each hydrogen may alternatively be prepared as described in Scheme 2, below.

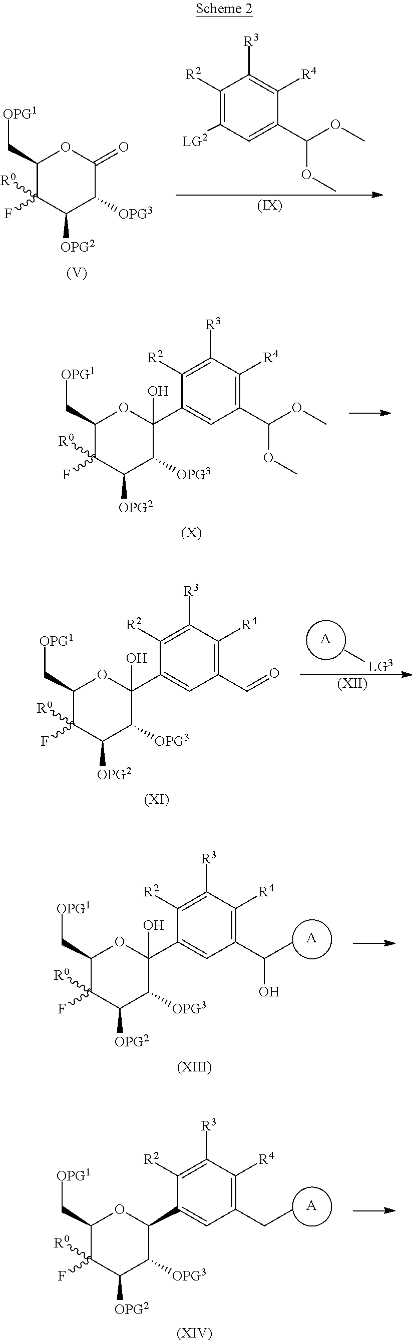

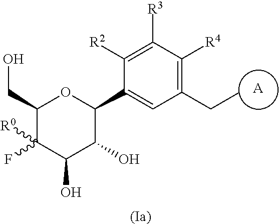

Accordingly, a suitably substituted compound of formula (V), for example a compound of formula (V) wherein PG¹, PG² an PG³ are each benzyl, is reacted with a suitably substituted compound of formula (IX), wherein LG² is a suitably selected leaving group such as Br, I, mesylate, tosylate, trifluoromethanesulfonyl, and the like, wherein the compound of formula (IX) is pre-treated with a suitably selected organolithium or Grignard reagent such as n-BuLi, s-BuLi, i-PrMgCl.LiCl, and the like; in a suitably selected organic solvent such as THF, diethyl ether, HMPA, and the like (to yield the corresponding organometallic salt); at a temperature less than about room temperature, preferably at about −78° C.; to yield the corresponding compound of formula (X).

The compound of formula (X) is reacted with a suitably selected acid such as HCl, diluted H₂SO₄, TFA, and the like; in a suitably selected organic solvent such as THF, diethyl ether, MeOH, and the like; to yield the corresponding compound of formula (XI).

The compound of formula (XI) is reacted with a suitably substituted, and optionally protected, compound of formula (XII), wherein LG³ is a suitably selected leaving group such as Br, I, mesylate, tosylate, trifluoromethanesulfonyl, and the like, wherein the compound of formula (XII) is pre-treated with a suitably selected organolithium reagent such as n-BuLi, sec-BuLi, t-BuLi, and the like (to yield the corresponding lithium salt); in a suitably selected organic solvent such as THF, diethyl ether, 2-methyl-THF, and the like; at a temperature less than about room temperature, preferably at about −78° C.; to yield the corresponding compound of formula (XIII).

The compound of formula (XIII) is reacted with a suitably selected reducing agent such as a mixture of Et₃SiH and BF₃.Et₂O, Et₃SiH and TFA, and the like; in a suitably selected organic solvent such as DCM, a mixture of DCM and acetonitrile, CHCl₃, THF, and the like; to yield the corresponding compound of formula (XIV).

The compound of formula (XIV) is de-protected according to known methods, or according to methods as described herein; to yield the corresponding compound of formula (Ia).

Compounds of formula (I) wherein R¹ is hydroxy-methyl- (i.e. —CH₂OH) may alternatively be prepared as described in Scheme 3, below.

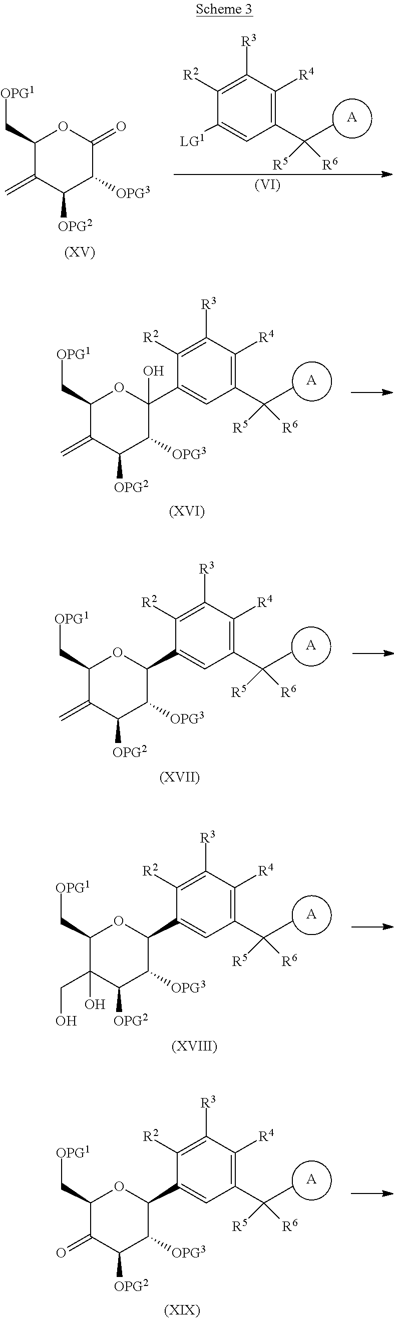

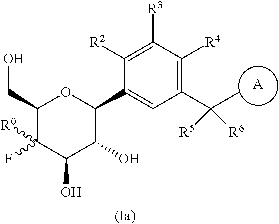

Accordingly, a suitably substituted compound of formula (XV), wherein PG¹, PG² and PG³ are each an independently selected oxygen protecting group such as benzyl, trimethylsilyl (TMS), t-butyl-dimethyl-silyl (TBDMS), t-butyl-diphenyl-silyl (TBDPS), and the like, a known compound or compound prepared by known methods is reacted with a suitably substituted compound of formula (VI); wherein LG is a suitably selected leaving group such as Br, Cl, I, mesylate, tosylate, trifluoromethanesulfonyl, and the like, wherein the compound of formula (VI) is pre-treated with a suitably selected organolithium or Grignard reagent such as n-BuLi, sec-BuLi, i-PrMgCl.LiCl, and the like (to yield the corresponding organometallic salt); in a suitably selected organic solvent such as THF, TMEDA (tetramethylethylenediamine), HMPA (hexamethylphosphoramide), and the like; at a temperature less than about room temperature, preferably at about −78° C.; to yield the corresponding compound of formula (XVI).

The compound of formula (XVI) is reacted with a suitably selected reducing agent such as a mixture of EtSiH and BF₃.Et₂O, Et₃SiH and TFA, and the like; in a suitably selected organic solvent such as DCM, a mixture of DCM and acetonitrile, CHCl₃, and the like; to yield the corresponding compound of formula (XVII).

The compound of formula (XVII) is reacted with a suitably selected oxidizing agent such as N-methyl-morpholine N-oxide, and the like; in the presence of a suitably selected co-oxidant such as OsO₄, OXONE® (i.e. potassium peroxymonosulfate), and the like; in a suitably selected organic solvent such as C(O)(CH₃)₂, acetonitrile, t-butanol, and the like; at a temperature in the range of from about 0° C. to about 40° C.; to yield the corresponding compound of formula (XVIII).

The compound of formula (XVIII) is reacted with a suitably selected oxidizing agent such as Martin's reagent (i.e Dess-Martin periodinane), NaIO₄, Pb(OAc)₄, and the like; in a suitably selected organic solvent such as CH₂Cl₂, THF, methanol, and the like; at a temperature in the range of from about 0° C. to about 25° C.; to yield the corresponding compound of formula (XIX).

The compound of formula (XIX) is then reacted (either di-fluorinated and de-protected or alkylated, fluorinated and de-protected) to yield the corresponding compound of formula (Ia), as described in Schemes 4 and 5 below.

Wherein the desired compound of formula (Ia) R⁰ is fluoro, the compound of formula (Ia) may be prepared as described in Scheme 4, below

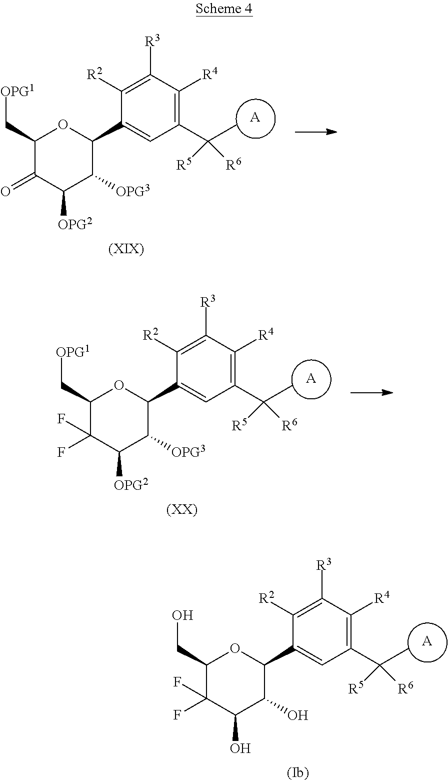

where the compound of formula (XIX) is reacted with a suitably selected source of fluoro such as BAST, DAST, morpholinosulfur trifluoride, and the like; in a suitably selected organic solvent such as DCM, benzene, CHCl₃, and the like; preferably at a temperature in the range of from about 0° C. to about room temperature; to yield the corresponding compound of formula (XX);

and the compound of formula (XX) is then de-protected, by reacting with a suitably selected Lewis acid such as BCl₃, and the like; in a suitably selected organic solvent such as DCM, and the like; at a temperature in the range of from about −78° C. to about 0° C.; to yield the corresponding compound of formula (Ib) (i.e. the corresponding compound of formula (Ia) wherein R⁰ is fluoro).

Wherein the desired compound of formula (Ia) R⁰ is is C₁₋₄alkyl (preferably methyl or ethyl), the compound of formula (Ia) may be prepared as described in Scheme 5, below

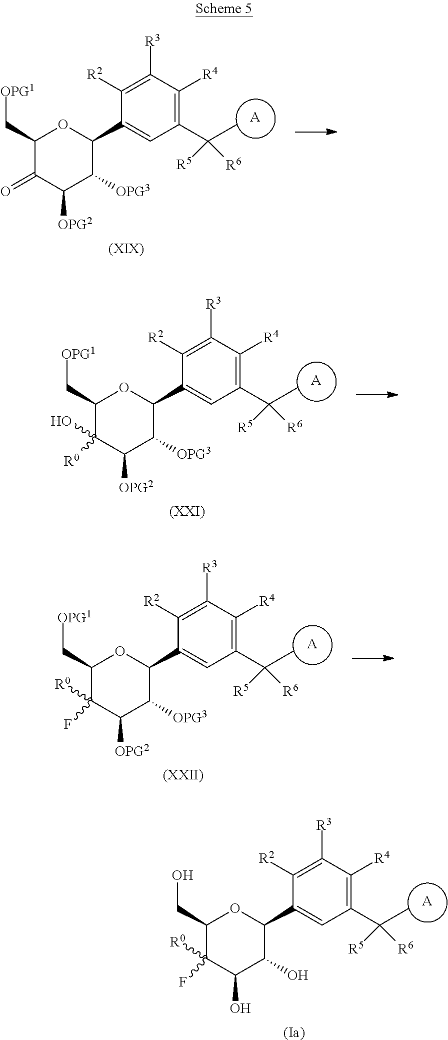

wherein the compound of formula (XIX) is reacted with a suitably selected alkylating agent, such as a C₁₋₄alkyl lithium, C₁₋₄alkyl MgBr, and the like; in a suitably selected organic solvent such as THF, diethyl ether, and the like; at a temperature in the range of from about −78° C. to about 0° C.; to yield the corresponding compound of formula (XXI); wherein R⁰ is the corresponding C₁₋₄ alkyl (for example wherein the alkylating agent is methyllithium, then R⁰ is methyl);

the compound of formula (XXI) is reacted with a suitably selected fluorinating agent such as BAST, DAST, morpholinosulfur trifluoride, and the like; in a suitably selected organic solvent such as DCM, CHCl₃, and the like; preferably at about room temperature; to yield the corresponding compound of formula (XXII);

and the compound of formula (XXII) is then de-protected by reacting with by reacting with a suitably selected Lewis acid such as BCl₃, and the like; in a suitably selected organic solvent such as DCM, and the like; at a temperature in the range of from about −78° C. to about 0° C.; to yield the corresponding compound of formula (Ia), wherein R⁰ is the corresponding C₁₋₄alkyl.

Compounds of formula (I) wherein R¹ is hydroxy substituted C₁₋₄alkyl (including, for example —CH₂OH, —CH(OH)CH₃, —C(OH)(CH₃)₂, and the like) may be prepared as described in Scheme 6, below.

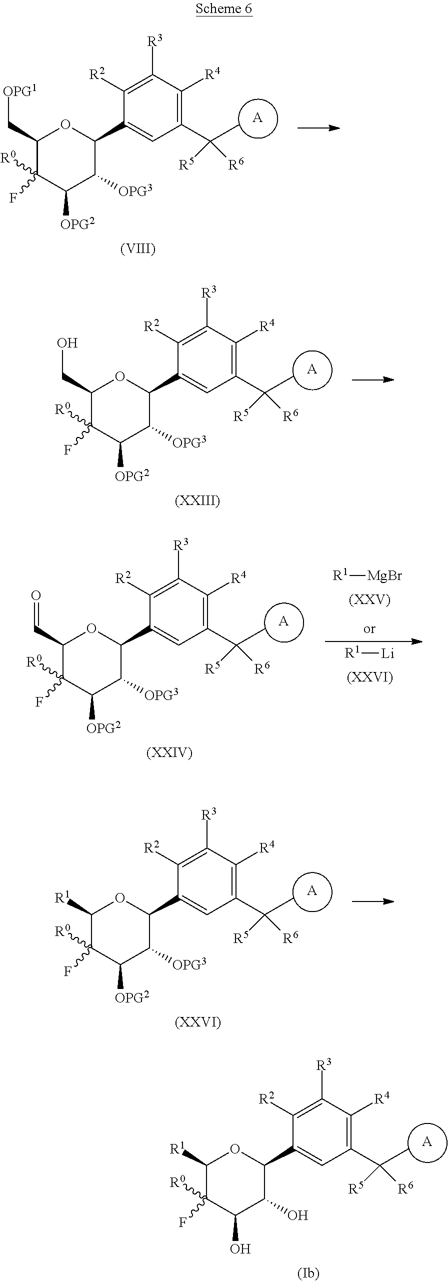

Accordingly, a suitably substituted compound of formula (VIII) (or a suitably substituted compound of formula (XIV), (XX) or (XXI)), prepared as described herein, is selectively de-protected according to known methods, to yield the corresponding compound of formula (XXIII). For example, wherein PG¹ benzyl, the compound of formula (VIII) is selectively deprotected under hydrogen atmosphere in the presence of catalyst such as 5% Pd on carbon, 10% Pd/C, 20% of Pd(OH)₂, and the like.

The compound of formula (XXIII) is reacted with a suitably selected oxidizing agent such as Dess-Martin reagent, DMSO/oxalyl chloride, PCC, and the like; in a suitably selected organic solvent such as 1,2-dichloroethane, chloroform, dichloromethane (DCM), and the like; to yield the corresponding compound of formula (XXIV) (where the hydroxymethyl (i.e. —CH₂OH) group is converted to the corresponding aldehyde (i.e —CH═O) group)

The compound of formula (XXIV) is reacted with a suitably selected substituted C₁₋₄alkylmagnesium bromide, a compound of formula (XXV) or a suitably selected substituted C₁₋₄alkyllithium, a compound of formula (XXVI), known compounds or compounds prepared by known methods, in a suitably selected organic solvent such as THF, 2-methyl tetrahydrofuran (2-Me-THF), diethyl ether, and the like; to yield the corresponding compound of formula (XXVII), where the aldehyde group is converted to the corresponding hydroxy-substituted C₁₋₄alkyl group.

The compound of formula (XXVII) is then de-protecting said compound as described herein, to remove the PG² and PG³ groups; to yield the compound of formula (Ib), wherein R¹ is hydroxy substituted C₁₋₄alkyl.

Compounds of formula (I) wherein R¹ and R^{1a} are taken together with the carbon atom to which they are bound to form cycloprop-1,1-diyl may be prepared as described in Scheme 7, below.

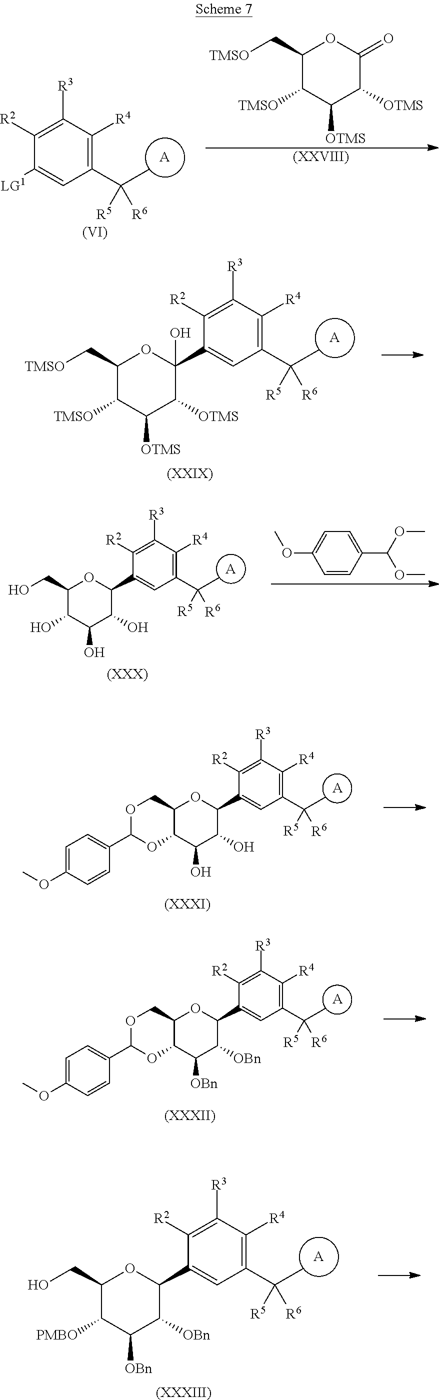

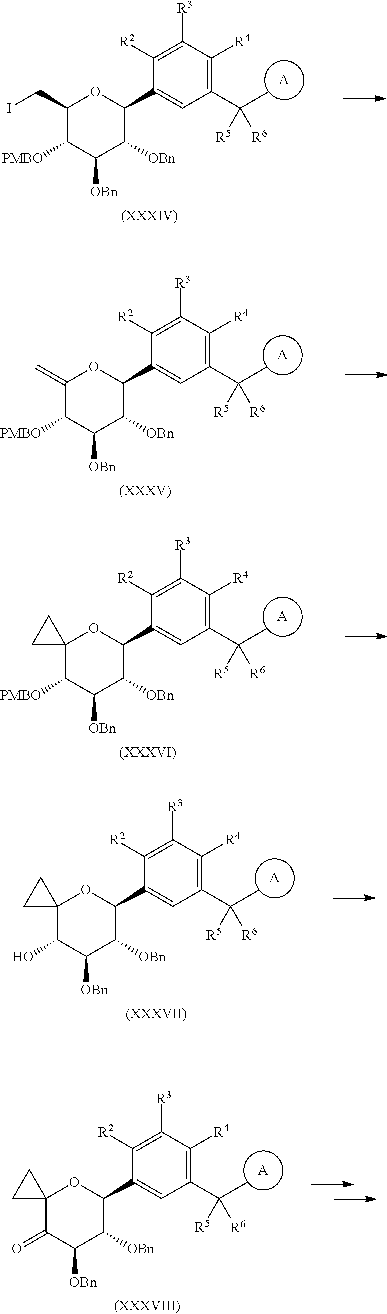

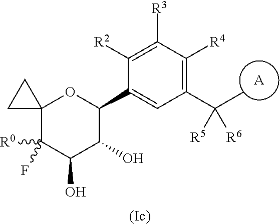

Accordingly, a compound of formula (XXVIII) (or the corresponding compound of formula (XXVIII) wherein the TMS protecting groups are replaced with TES protecting groups) is reacted with a suitably substituted a suitably substituted compound of formula (VI); wherein LG¹ is a suitably selected leaving group such as Br, Cl, I, mesylate, tosylate, trifluoromethanesulfonyl, and the like, wherein the compound of formula (VI) is pre-treated with a suitably selected organolithium or Grignard reagent such as n-BuLi, s-BuLi, i-PrMgCl.LiCl, and the like (to yield the corresponding organometallic salt); in a suitably selected organic solvent such as THF, TMEDA (tetramethylethylenediamine), HMPA (hexamethylphosphoramide), and the like; at a temperature less than about room temperature, preferably at about −78° C.; to yield the corresponding compound of formula (XXIX).

The compound of formula (XXIX) is reacted with a suitably selected reducing agent such as a mixture of Et₃SiH and BF₃.Et₂O, Et₃SiH and TFA, and the like; in a suitably selected organic solvent such as DCM, a mixture of DCM and acetonitrile, CHCl₃, and the like; to yield the corresponding compound of formula (XXX).

The compound of formula (XXX) is reacted with 1-(dimethoxymethyl)-4-methoxybenzene, a known compound; in the presence of a catalytic amount of a suitably selected acid (including, but not limited to a suitably selected Lewis acid) such as PPTS, D-camphorsulfonic acid, Cu(CF₃SO₃)₂, and the like; in a suitably selected organic solvent such as DMF, acetonitrile, CHCl₃, and the like; preferably at room temperature; to yield the corresponding compound of formula (XXXI).

The compound of formula (XXXI) is reacted with a suitably selected protecting agent, preferably benzylbromide, a known compound; in the presence of a suitably selected base such as NaH, K[N(Si(CH₃)₃)₂], and the like; in a suitably selected organic solvent such as DMF, THF, toluene, and the like; to yield the corresponding compound of formula (XXXII).

The compound of formula (XXXII) is reacted with a suitably selected reducing agent such as DIBAL-H, LiAlH₄/AlCl₃, NaBH₃CN/Me₃SiH, and the like; in a suitably selected organic solvent such as DCM, THF, diethyl ether, and the like; at a temperature in the range of from about −78° C. to about −20° C.; to yield the corresponding compound of formula (XXXIII).

The compound of formula (XXXIII) is reacted with a suitably selected source of iodine such as 12, and the like; in the presence of a suitably selected coupling agent such as PPh₃, and the like; preferably in the presence of imidazole; in a suitably selected organic solvent such as toluene, THF, acetonitrile, and the like; at a temperature in the range of from about 0° C. to about 80° C., for example, at about 70° C.; to yield the corresponding compound of formula (XXXIV).

The compound of formula (XXXIV) is reacted with a suitably selected base such as DBU, t-BuOK, NaH, and the like; in a suitably selected organic solvent such as DMF, THF, and the like; at a temperature in the range of from about 0° C. to about 80° C., for example, at about 70° C.; to yield the corresponding compound of formula (XXXV).

The compound of formula (XXXV) is reacted (under Smith-Simmons reaction conditions) with a suitably selected organozinc reagent such as diethylzinc, and the like; in the presence of a suitably selected dihaloalkane such as CH₂I₂, ClCH₂I, and the like; in a suitably selected organic solvent such as DCM, hexane, ClCH₂CH₂Cl, and the like; at a temperature in the range of from about 0° C. to about reflux temperature, for example, at about reflux temperature; to yield the corresponding compound of formula (XXXVI).

The compound of formula (XXXVI) is reacted with a suitably selected oxidizing agent such as DDQ, Ce(NH₄)₂(NO₃)₆, and the like or a suitably selected acid such as TFA, and the like; in the presence of a suitably selected reducing agent such as Et₃SiH, and the like; in a suitably selected organic solvent such as DCM, acetonitrile, and the like; to yield the corresponding compound of formula (XXXVII).

The compound of formula (XXXVII) is reacted with a suitably selected oxidizing agent such as Dess-Martin periodinane, PCC, DMSO/oxalyl chloride, and the like; in a suitably selected organic solvent such as DCM, CHCl₃, and the like; to yield the corresponding compound of formula (XXXVIII).

The compound of formula (XXXVIII) is reacted as described in Scheme 5, to yield the corresponding compound of formula (Ic) (i.e. the corresponding compound of formula (Ib) wherein R¹ and R^{1a} are taken together as cycloprop-1,1-diyl). More particularly, the compound of formula (XXXVIII) may be substituted for the compound of formula (XIX) in Scheme 4 and di-fluorinated and de-protected as therein described, to yield the corresponding compound of formula (I) wherein R⁰ is fluoro and R¹ and R^{1a} are taken together as cycloprop-1,1-diyl.

Compounds of formula (V) wherein R⁰ is fluoro may be prepared as described in Scheme 8, below.

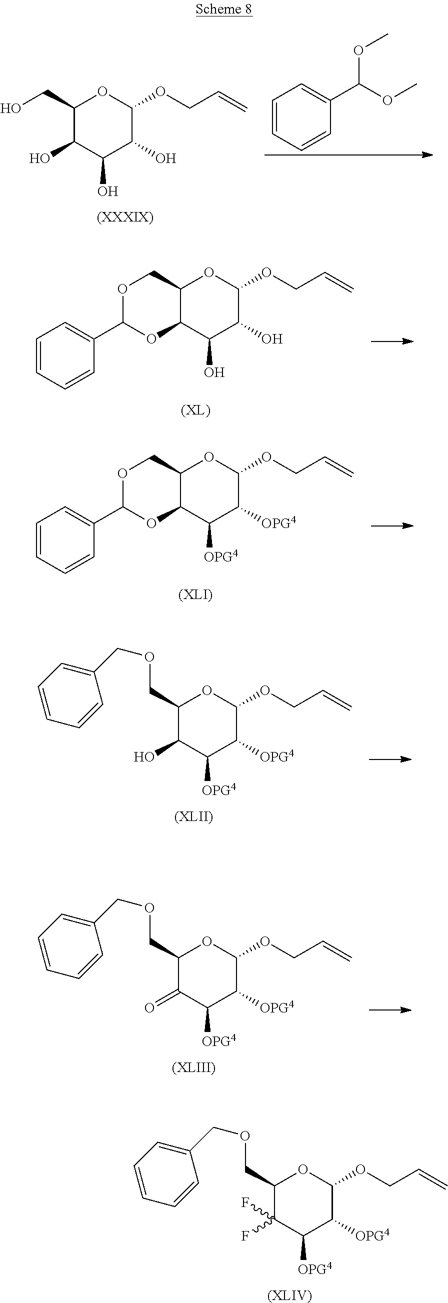

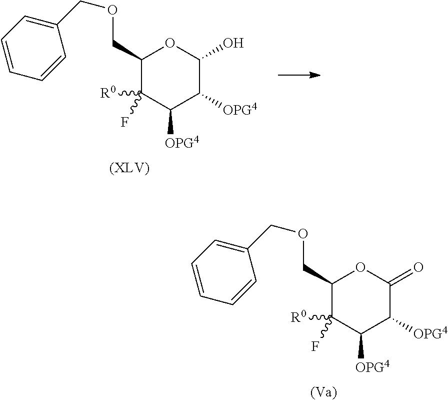

Accordingly, a suitably substituted compound of (XXXIX), a known compound or compound prepared by known methods is reacted with (dimethoxymethyl)benzene, a known compound; in the presence of a catalytic amount of an acid such as D-camphorsulfonic acid (CSA), p-CH₃C₆H₄SO₃H, HBF₄, and the like; in a suitably selected organic solvent such as chloroform, CH₂Cl₂, DMF, acetonitrile, toluene, and the like; at a temperature in the range of from about 0° C. to about 80° C., preferably at about reflux temperature; to yield the corresponding compound of formula (XL).

The compound of formula (XL) is protected, according to known methods, to yield the corresponding compound of formula (XLI), wherein each PG⁴ is the corresponding oxygen protecting group such as benzyl, p-methoxybenzyl (PMB), and the like. For example, the compound of formula (XL) is reacted with a suitably selected source of benzyl such as benzyl bromide, PMB-Cl, and the like; in the presence of a suitably selected base such as NaH, KH, KOH, and the like; in a suitably selected organic solvent such as DMF, THF, toluene, and the like; preferably at about room temperature; to yield the corresponding compound of formula (XLI), wherein each PG⁴ is benzyl.

The compound of formula (XLI) is reacted with a suitably selected reducing agent(s) such as NaBH₃CN, TFA/EtSiH, BH₃.THF/Cu(CF₃SO₃)₂, Et₃SiH/I₂, and the like; in a suitably selected organic solvent such as THF, acetonitrile, CH₂Cl₂, and the like; preferably at about room temperature; to yield the corresponding compound of formula (XLII).

The compound of formula (XLII) is reacted with a suitably selected oxidizing agent such as acetic anhydride/DMSO, PCC, Martin's reagent, and the like; in a suitably selected organic solvent such as acetonitrile, CH₂Cl₂, and the like; at a temperature in the range of from about −20° C. to about 25° C.; to yield the corresponding compound of formula (XLIII).

The compound of formula (XLIII) is reacted with a suitably selected source of fluoro such as BAST, DAST, and the like; in a suitably selected organic solvent such as DCM, CHCl₃, and the like; preferably at about room temperature; to yield the corresponding compound of formula (XLIV).

The compound of formula (XLIV) is reacted with a suitably base such as sodium acetate, NaOBu-t, t-BuOK, and the like; in the presence of a suitably selected reagent such as PdCl₂, HgO/HgCl₂, and the like; in a suitably selected organic solvent such as acetic acid, C(O)(CH₃)₂, DMSO, and the like; to yield the corresponding compound of formula (XLV). Alternatively, the compound of formula (XLIV) is reacted with a suitably selected reducing agent such as n-Bu₃SnH, and the like; in the presence of a suitably selected catalyst such as Pd(PPh₃)₄, and the like; optionally in the presence ZnCl₂, and the like; in a suitably selected solvent such as THF, 2-methyl-THF, toluene, and the like; preferably at about room temperature; to yield the corresponding compound of formula (XLV).

The compound of formula (XLV) is reacted with a suitably selected oxidizing agent such as acetic anhydride/DMSO, PCC, MnO₂, and the like; in a suitably selected organic solvent such as, CH₂Cl₂, AcOEt, and the like; at a temperature in the range of from about 0° C. to about 25° C.; to yield the corresponding compound of formula (Va).

Compounds of formula (V) wherein R⁰ is C₁₋₄alkyl (preferably methyl or ethyl) may be prepared as described in Scheme 9, below.

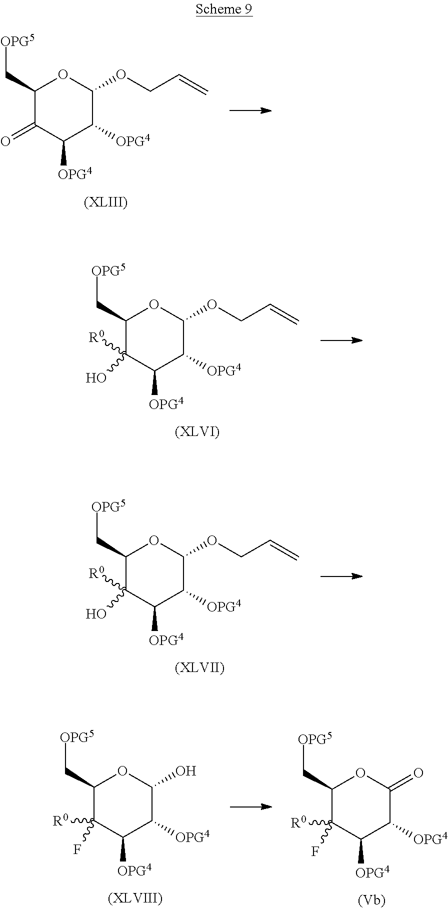

Accordingly, a suitably substituted compound of formula (XLIII), prepared for example as described in Scheme 8 above, is reacted with a suitably selected alkylating agent, such as a C₁₋₄ alkyl lithium, C₁₋₄ alkyl MgBr, and the like; in a suitably selected organic solvent such as THF, diethyl ether, and the like; at a temperature in the range of from about −78° C. to about 0° C.; to yield the corresponding compound of formula (XLVI); wherein R⁰ is the corresponding C₁₋₄alkyl (for example wherein the alkylating agent is methyliodide, then R⁰ is methyl).

The compound of formula (XLVI) is reacted with a suitably selected fluorinating agent such as BAST, DAST, 4-tert-butyl-2,6-dimethylphensulfurtrifluoride, HF-pyridine, and the like; in a suitably selected organic solvent such as DCM, toluene, ClCH₂CH₂Cl, and the like; preferably at about room temperature; to yield the corresponding compound of formula (XLVII).

The compound of formula (XLVII) is reacted with a suitably base such as sodium acetate, NaOBu-t, t-BuOK, and the like; in the presence of a suitably selected reagent such as PdCl₂, HgO/HgCl₂, and the like; in a suitably selected organic solvent such as acetic acid, C(O)(CH₃)₂, DMSO, and the like; to yield the corresponding compound of formula (XLVIII). Alternatively, the compound of formula (XLVII) is reacted with a suitably selected reducing agent such as n-Bu₃SnH, and the like; in the presence of a suitably selected catalyst such as Pd(PPh₃)₄, and the like; optionally in the presence ZnCl₂, and the like; in a suitably selected solvent such as THF, 2-methyl-THF, toluene, and the like; preferably at about room temperature; to yield the corresponding compound of formula (XLVIII).

The compound of formula (XLVIII) is reacted with a suitably selected oxidizing agent such as acetic anhydride/DMSO, PCC, MnO₂, and the like; in a suitably selected organic solvent such as, CH₂Cl₂, EtOAc, and the like; at a temperature in the range of from about 0° C. to about 25° C.; to yield the corresponding compound of formula (Vb).

One skilled in the art will recognize that compounds of formula (VI)

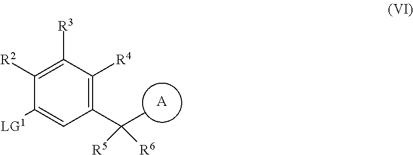

are known compounds, compounds which may be prepared by known methods or compounds which may be prepared according to the methods as described in the Schemes and Examples herein.

Compounds of formula (VI), particularly compounds of formula (VI) wherein R² and R³ or R³ and R⁴ are taken together with the carbon atoms to which they are bound to form a ring structure as herein defined may be prepared as described in Scheme 10, below.

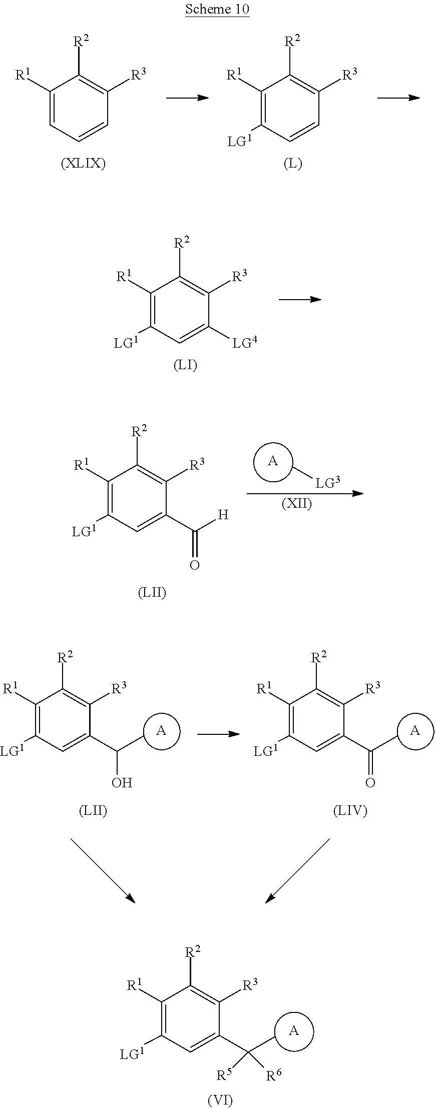

Accordingly, a suitably substituted compound of formula (XLIX), a known compound or compound prepared by known methods, is reacted with a first suitably selected halogenating reagent such as Br₂, NBS, and the like; in a suitably selected organic solvent such as DCM, THF, CH₃CN, and the like; to yield the corresponding compound of formula (L).

The compound of formula (L) is reacted with a second suitably selected halogenating reagent such as 12, NIS, and the like; wherein the first and second halogenating agents are selected such that the attached first and second halogen substituent groups (LG¹ and LG⁴ respectively) are orthogonal for metal-halogen exchange reaction; in a suitably selected organic solvent such as DCM, THF, CH₃CN and the like; to yield the corresponding compound of formula (LI).

The compound of formula (LI) is reacted with a suitably selected organolithium or Grignard reagent such as n-BuLi, sec-BuLi, i-PrMgCl.LiCl, and the like and the like; and the resulting intermediate (which is not isolated) is then immediately reacted with DMF; to yield the compound of formula (LII).

The compound of formula (XII), wherein LG³ is a suitably selected leaving group such as Br, Cl, I, and the like, is pre-treated (and admixed with) a suitably selected base such as n-BuLi, t-BuLi, sec-BuLi and the like (to yield the corresponding lithium salt); and then reacted with the compound of formula (LII); in a suitably selected organic solvent such as THF, diethyl ether, and the like; at a temperature in the range of from about −78° C. to about 20° C., preferably at about −78° C.; to yield the corresponding compound of formula of (LIII).

The compound of formula (LIII) is reacted with a suitably selected reducing agent such as triethylsilane, LAH, and the like; in the presence of suitably selected Lewis acid such as BF₃.Et₂O, TFA, AlCl₃, and the like; in a suitably selected organic solvent such as DCM, diethyl ether, and the like; to yield compound formula (VI), wherein R⁵ and R⁶ are each H.

Alternatively, the compound of formula (LIII) is reacted with a suitably selected oxidizing reagent such as PCC, MnO₂, Dess-Martin reagent, and the like; in a suitably selected organic solvent such as DCM, 1,2-dicholorethane, DMSO and the like; to yield the compound of formula (LIV).

The compound of formula (LIV) is reacted with a suitably selected reducing agent such as deuterium-substituted triethylsilane (triethylsilane-d4), deuterium-substituted LAH, and the like; in the presence of suitably selected Lewis acid such as BF₃.Et₂O, AlCl₃, and the like; in a suitably selected organic solvent such as DCM, diethyl ether, and the like; to yield compound formula (VI), wherein R⁵ and R⁶ are each deuterium.

Compounds of formula (VI), particularly compounds of formula (VI) wherein R², R³ and R⁴ are each independently a substituent group may be prepared as described in Scheme 11, below.

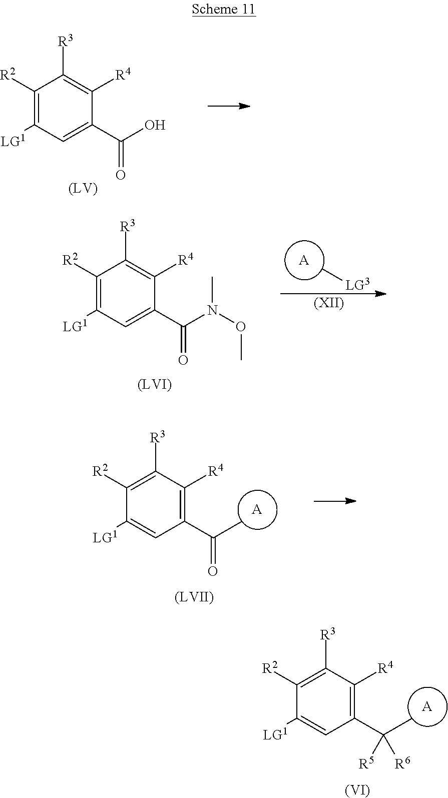

Accordingly, a suitably substituted compound of formula (LV), a known compound or compound prepared by known methods, is reacted with for example, a suitably selected coupling reagent such as HATU, EDCl/HOBt, and the like; in the presence of a suitably selected base such as Et₃N, DIPEA, and the like; in a suitably selected organic solvent such as DCM, DMF, and the like; to yield the corresponding compound of formula (LVI) (wherein the carboxylic acid substituent group on the compound of formula (LV) is converted to a Weinreb amide).

The compound of formula (XII), wherein LG³ is a suitably selected leaving group such as Br, I, and the like is pre-treated (and admixed with) a suitably selected base such as n-BuLi, t-BuLi, sec-BuLi and the like (i.e. to yield the corresponding lithium salt); in a suitably selected organic solvent such as THF, diethyl ether, and the like; at a temperature in the range of from about −78° C. to about 20° C., preferably at a temperature of about −78° C.; and then reacted with a suitably substituted compound of formula (LVI); a known compound or compound prepared by known methods, to yield the corresponding compound of formula (LVII).

The compound of formula (LVII) is reacted with a suitably selected reducing agent such as triethylsilane, LAH, and the like; in the presence of suitably selected Lewis acid such as BF₃.Et₂O, TFA, AlCl₃, and the like; in a suitably selected organic solvent such as DCM, diethyl ether, and the like; to yield the corresponding compound of formula compound formula (VI), wherein R⁵ and R⁶ are each hydrogen.

Alternatively, the compound of formula (LVII) is reacted with a suitably selected reducing agent such as deuterium-substituted triethylsilane (triethylsilane-d₄), deuterium-substituted LAH, and the like; in the presence of suitably selected Lewis acid such as BF₃.Et₂O, TFA, AlCl₃, and the like; in a suitably selected organic solvent such as DCM, diethyl ether, and the like; to yield the corresponding compound of formula (VI), wherein R⁵ and R⁶ are each deuterium.

### Pharmaceutical Compositions:

The present invention further comprises pharmaceutical compositions containing one or more compounds of formula (I) with a pharmaceutically acceptable carrier. Pharmaceutical compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions of this invention, one or more compounds of the present invention as the active ingredient is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, of from about 0.01 mg to about 1000 mg or any amount or range therein, and may be given at a dosage of from about 0.05 mg/day to about 300 mg/day, or any amount or range therein, preferably from about 0.1 mg/day to about 100 mg/day, or any amount or range therein, preferably from about 1 mg/day to about 50 mg/day, or any amount or range therein. The dosages, however, may be varied depending upon the requirement of the patients, the severity of the condition being treated and the compound being employed. The use of either daily administration or post-periodic dosing may be employed.

Preferably these compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from about 0.01 mg to about 1,000 mg, or any amount or range therein, of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of the present invention may be incorporated for administration orally or by injection include, aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The method of treating disorders mediated by SGLT activity, preferably dual SGLT1 and SGLT2 activity, described in the present invention may also be carried out using a pharmaceutical composition comprising any of the compounds as defined herein and a pharmaceutically acceptable carrier. The pharmaceutical composition may contain between about 0.01 mg and about 1000 mg of the compound, or any amount or range therein, preferably from about 0.05 mg to about 300 mg of the compound, or any amount or range therein, more preferably from about 0.1 mg to about 100 mg of the compound, or any amount or range therein, more preferably from about 0.1 mg to about 50 mg of the compound, or any amount or range therein; and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixirs, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitably flavored suspending or dispersing agents such as the synthetic and natural gums, for example, tragacanth, acacia, methylcellulose and the like. For parenteral administration, sterile suspensions and solutions are desired. Isotonic preparations which generally contain suitable preservatives are employed when intravenous administration is desired.

To prepare a pharmaceutical composition of the present invention, a compound of formula (I) as the active ingredient is intimately admixed with a pharmaceutical carrier according to conventional pharmaceutical compounding techniques, which carrier may take a wide variety of forms depending of the form of preparation desired for administration (e.g. oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

Compounds of this invention may be administered in any of the foregoing compositions and according to dosage regimens established in the art whenever treatment of disorders mediated by SGLT activity, preferably dual SGLT1 and SGLT2 activity, is required.

The daily dosage of the products may be varied over a wide range from about 0.01 mg to about 1,000 mg per adult human per day, or any amount or range therein. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250 and 500 milligrams of the active ingredient for the symptomatic adjustment of the dosage to the patient to be treated. An effective amount of the drug may be ordinarily supplied at a dosage level of from about 0.005 mg/kg to about 10 mg/kg of body weight per day, or any amount or range therein. Preferably, the range is from about 0.01 to about 5.0 mg/kg of body weight per day, or any amount or range therein, more preferably, from about 0.1 to about 1.0 mg/kg of body weight per day, or any amount or range therein, more preferably, from about 0.1 to about 0.5 mg/kg of body weight per day, or any amount or range therein. The compounds may be administered on a regimen of 1 to 4 times per day.

Optimal dosages to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trails including first-in-human, dose ranging and efficacy trials, in healthy patients and/or those suffering from a given disorder, may be completed according to methods well known in the clinical and medical arts.

The following Examples are set forth to aid in the understanding of the invention, and are not intended and should not be construed to limit in any way the invention set forth in the claims which follow thereafter.

In the Examples which follow, some synthesis products are listed as having been isolated as a residue. It will be understood by one of ordinary skill in the art that the term “residue” does not limit the physical state in which the product was isolated and may include, for example, a solid, an oil, a foam, a gum, a syrup, and the like.

Examples A and B which follow herein describe representative small scale and scale-up procedures for the preparation of the title compounds. Both the small scale and scale-up procedures were used (as disclosed below) in the preparation of at least one batch of the title intermediate compound.

### Example A: (Small Scale Intermediate Synthesis)

### (3R,4R,6R)-3,4-bis(benzyloxy)-6-((benzyloxy)methyl)-5,5-difluorotetrahydro-2H-pyran-2-one

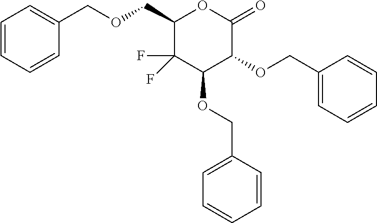

### Step 1: Preparation of (4aR,6S,7R,8R,8aR)-6-(allyloxy)-2-phenylhexahydropyrano[3,2-d][1,3]dioxine-7,8-diol

Small-scale synthesis: To a solution of allyl alpha-D-galactopyranoside (1.0 g, 4.51 mmol) in acetonitrile (25 ml) was added p-methoxybenzaldehyde dimethyl acetal (1.65 g, 9.08 mmol) followed by CSA (105 mg, 0.45 mmol). After 20 h, Et₃N (0.1 ml) was added and the solvent was removed under reduced pressure and the residue was partitioned between EtOAc and water. The aqueous layer was extracted with EtOAc three times and the combined organic extracts were washed with brine, dried with Na₂SO₄, filtered and concentrated. The residue was purified by flash column chromatography on silica gel (40 g column, EtOAc/heptane: 0>>>20%>>>100%, Teledyne ISCO Combiflash) to yield a white solid.

### Step 2: Preparation of (4aR,6S,7R,8S,8aS)-6-(allyloxy)-7,8-bis(benzyloxy)-2-phenylhexahydropyrano[3,2-d][1,3]dioxine

Small-scale synthesis (Batch C): To a mixture of (4aR,6S,7R,8R,8aR)-6-(allyloxy)-2-phenylhexahydropyrano[3,2-d][1,3]dioxine-7,8-diol (242.2 mg, 0.79 mmol), sodium hydride (125.7 mg, 3.14 mmol) and n-Bu₄NI (290.1 mg, 0.79 mmol) was added 8 ml of anhydrous DMF under argon atmosphere at room temperature and the resulting mixture was stirred at room temperature for 20 min. Neat benzyl bromide (0.30 ml, 2.51 mmol) was added and the reaction mixture was stirred at room temperature for 20 hours. The reaction was quenched with aq. NH₄Cl solution and extracted with EtOAc three times. The combined organic layers were washed with brine, dried with Na₂SO₄, filtered and concentrated. The residue was then purified by flash column chromatography on silica gel (12 g Combiflash column, EtOAc/heptane: 0>>>5%>>>15%) to yield a white solid. ¹H NMR (CHLOROFORM-d) δ: 7.49-7.55 (m, 2H), 7.26-7.44 (m, 13H), 5.86-5.99 (m, 1H), 5.48 (s, 1H), 5.28-5.35 (m, 1H), 5.21 (dd, J=10.6, 1.5 Hz, 1H), 4.97 (d, J=3.5 Hz, 1H), 4.81-4.89 (m, 2H), 4.72-4.77 (m, 1H), 4.67 (d, J=11.6 Hz, 1H), 4.12-4.24 (m, 3H), 3.97-4.11 (m, 4H), 3.64 (s, 1H). LC-MS (ES, m/z): 510.9 [M+Na]⁺

### Step 3: Synthesis of (2R,3S,4S,5R,6S)-6-(allyloxy)-4,5-bis(benzyloxy)-2-((benzyloxy)methyl)tetrahydro-2H-pyran-3-ol

(4aR,6S,7R,8S,8aS)-6-(allyloxy)-7,8-bis(benzyloxy)-2-phenylhexahydropyrano[3,2-d][1,3]dioxine (156 mg, 0.32 mmol) was dissolved in a mixture of dichloromethane and toluene (1 ml, 1/3 v/v) and then evaporated to dryness and further dried in vacuo for 2 h. The resulting residue was then dissolved in anhydrous THE (4 ml), followed by addition of MS (4 Å, 243 g). To the above suspension was added sodium cyanoborohydride (301.6 mg, 4.8 mmol). After stirring at 0° C. for 20 mins, a 1.0 M solution of HCl in diethyl ether (4.15 ml) was added dropwise and the reaction mixture was stirred at room temperature for 18 h then diluted with EtOAc, washed with saturated aqueous NaHCO₃ solution, water and brine. It was dried with Na₂SO₄, filtered and concentrated to yield a syrup, which was purified by flash column chromatography on silica gel (12 g RediSep Rf column, EtOAc/heptane: 0>>>5%>>>15%) to yield a colorless syrup (65.3 mg, yield: 41.7%). ¹H NMR (CHLOROFORM-d) δ: 7.30-7.42 (m, 15H), 5.94 (ddt, J=16.7, 11.1, 5.7 Hz, 1H), 5.31 (br d, J=17.4 Hz, 1H), 5.20 (br d, J=10.3 Hz, 1H), 4.87 (br s, 1H), 4.80 (br d, J=11.2 Hz, 2H), 4.63-4.74 (m, 2H), 4.53-4.62 (m, 2H), 4.15 (br d, J=4.9 Hz, 1H), 4.00-4.11 (m, 2H), 3.96 (br t, J=5.4 Hz, 1H), 3.85-3.92 (m, 2H), 3.63-3.78 (m, 2H). LC-MS (ES, m/z): 508.2 [M+NH₄]⁺

### Step 4: Preparation of (2R,4R,5R,6S)-6-(allyloxy)-4,5-bis(benzyloxy)-2-((benzyloxy)methyl)dihydro-2H-pyran-3(4H)-one

To a solution of (2R,3S,4S,5R,6S)-6-(allyloxy)-4,5-bis(benzyloxy)-2-((benzyloxy)methyl)tetrahydro-2H-pyran-3-ol (1.76 g, 2.87 mmol) in dichloromethane (50 mL) was added pyridinium chlorochromate (2.47 g, 11.5 mmol) in portions and the mixture was stirred at room temperature for 3 days. The mixture was passed through a pad of CELITE®, washed with dichloromethane and the filtrate was concentrated under reduced pressure. The residue was purified by flash column chromatography on silica gel (EtOAc/heptane: 0>>>15%) to yield a colorless syrup. LC-MS (ES, m/z): 506.4 [M+NH₄]⁺

### Step 5: Preparation of (2R,4R,5R,6S)-6-(allyloxy)-4,5-bis(benzyloxy)-2-((benzyloxy)methyl)-3,3-difluorotetrahydro-2H-pyran

Bis-(2-methoxyethyl)amino sulfur trifluoride (BAST or Deoxy-Fluoro®, 0.78 ml, 4.23 mmol) was added dropwise to (2R,4R,5R,6S)-6-(allyloxy)-4,5-bis(benzyloxy)-2-((benzyloxy)methyl)dihydro-2H-pyran-3(4H)-one (939.7 mg, 1.92 mmol) in DCM (10 ml) in a 100 ml plastic bottle and the mixture was stirred at room temperature for 18 h. Aqueous NaHCO₃ solution was slowly added and the organic layer was separated. The aqueous layer was extracted with dichloromethane three times and the combined organic layer was washed with brine, dried with Na₂SO₄, filtered, and concentrated to yield a yellow syrup, which was purified by flash column chromatography on silica gel (80 g column, Teledyne ISCO/Combiflash, EtOAc/heptane: 0>>>10%) to yield a colorless syrup. ¹H NMR (CHLOROFORM-d) δ: 7.36-7.44 (m, 2H), 7.24-7.35 (m, 13H), 5.88-6.00 (m, 1H), 5.28-5.36 (m, 1H), 5.20-5.25 (m, 1H), 4.76-4.91 (m, 4H), 4.50-4.65 (m, 3H), 4.01-4.23 (m, 4H), 3.87 (dd, J=10.9, 1.8 Hz, 1H), 3.62-3.73 (m, 2H). LC-MS (ES, m/z): 532.95 [M+Na]⁺

### Step 6: Preparation of (3R,4R,6R)-3,4-bis(benzyloxy)-6-((benzyloxy)methyl)-5,5-difluorotetrahydro-2H-pyran-2-ol

To a solution of (2R,4R,5R,6S)-6-(allyloxy)-4,5-bis(benzyloxy)-2-((benzyloxy)methyl)-3,3-difluorotetrahydro-2H-pyran (665.4 mg, 1.3 mmol) in 95% aq. HOAc (7.95 ml) was added NaOAc (534.5 mg, 6.52 mmol) and PdCl₂ (1155 mg, 6.52 mmol). The mixture was then stirred at ambient temperature for 24 h, filtered through a CELITE® pad, diluted with EtOAc, then washed with H₂O, aq. NaHCO₃, brine and dried with Na₂SO₄. The solvent was evaporated under reduced pressure to yield a brown syrup, which was purified by flash column chromatography on silica gel (12 g column, EtOAc/heptane: 0>>>15%) to yield a white solid. LC-MS (ES, m/z): 492.90 [M+Na]⁺

### Step 7: Preparation of (3R,4R,6R)-3,4-bis(benzyloxy)-6-((benzyloxy)methyl)-5,5-difluorotetrahydro-2H-pyran-2-one

To a solution of (3R,4R,6R)-3,4-bis(benzyloxy)-6-((benzyloxy)methyl)-5,5-difluorotetrahydro-2H-pyran-2-ol (1.0 g, 2.13 mmol) in DMSO (6 ml) was added acetic anhydride and the reaction mixture was stirred at room temperature for 3 days. Water (20 mL) was added and stirred for 1 h. EtOAc was added and the organic layer was washed with 1M NaHCO₃ solution three times and dried with Na₂SO₄. The combined organic layer was concentrated under reduced pressure and the residue was purified by flash column chromatography on silica gel (EtOAc/heptane: 0>>>10%, 40 g column) to yield a colorless syrup, which later became a white solid.

¹H NMR (CHLOROFORM-d) δ: 7.41-7.26 (m, 15H), 5.00 (d, J=11.12 Hz, 1H), 4.84 (d, J=11.62, 1H), 4.75-4.53 (m, 5H), 4.22 (dd, J=7.58 and 2.02 Hz, 1H), 4.01 (dd, J=15.66 and 8.08 Hz, 1H), 3.94 (dd, J=11.12 and 3.03 Hz, 1H), 3.79 (dd, J=11.12 and 6.57 Hz, 1H). LC-MS (ES, m/z): 491.25 [M+Na]⁺

### Example B: (Scale-Up Intermediate Synthesis)

### (3R,4R,6R)-3,4-bis(benzyloxy)-6-((benzyloxy)methyl)-5,5-difluorotetrahydro-2H-pyran-2-one

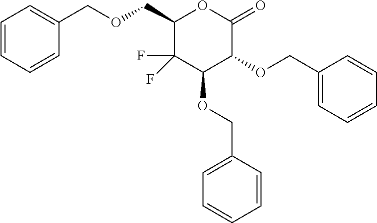

### Step 1: Preparation of (4aR,6S,7R,8R,8aR)-6-(allyloxy)-2-phenylhexahydropyrano[3,2-d][1,3]dioxine-7,8-diol

Into a 3-L 3-necked round-bottom flask purged and maintained with an inert atmosphere of nitrogen, was placed (2R,3R,4S,5R,6S)-2-(hydroxymethyl)-6-(prop-2-en-1-yloxy)oxane-3,4,5-triol (80 g, 363.27 mmol, 1.00 equiv), CAS (250 mg), (dimethoxymethyl)benzene (82 g, 538.80 mmol, 1.48 equiv), CHCl₃ (1.2 L). The resultant suspension was placed in a preheated oil bath (bath temp. 90° C.), and the distillate was collected. After approximately 15 mL was collected, the same volume of CHCl₃ was added. This process was repeated. The reaction progress was monitored by TLC. The resulting mixture was washed with water (1×500 mL) and saturated aqueous sodium bicarbonate (1×500 mL). The resulting mixture was washed with brine (1×500 mL). The mixture was dried over anhydrous sodium sulfate and concentrated under vacuum. The residue was washed with PE/EA (20:1) to yield (4aR,6S,7R,8R,8aR)-2-phenyl-6-(prop-2-en-1-yloxy)-hexahydro-2H-pyrano[3,2-d][1,3]dioxine-7,8-diol as a white solid. ¹H NMR: (300 MHz, CDCl₃): δ 7.54-7.47 (m, 2H), 7.41-7.34 (m, 3H), 5.99-5.86 (m, 1H), 5.55 (s, 1H), 5.34-5.21 (m, 2H), 5.09 (s, 1H), 4.29-4.21 (m, 3H), 4.11-4.05 (m, 2H), 3.93 (s, 2H), 3.74 (s, 1H). LC-MS (ES, m/z): 634.2 [2M+NH₄]⁺

### Step 2: Preparation of (4aR,6S,7R,8S,8aS)-6-(allyloxy)-7,8-bis(benzyloxy)-2-phenylhexahydropyrano[3,2-d][1,3]dioxine

Into a 3-L 3-necked round-bottom flask purged and maintained with an inert atmosphere of nitrogen, was placed (4aR,6S,7R,8R,8aR)-2-phenyl-6-(prop-2-en-1-yloxy)-hexahydro-2H-pyrano[3,2-d][1,3]dioxine-7,8-diol (100 g, 324.33 mmol, 1.00 equiv), N,N-dimethylformamide (1 L). This was followed by the addition of sodium hydride (39 g, 1.62 mol, 3.00 equiv, 60%), in portions at 0-10° C. The resulting solution was stirred for 30 minutes. To the mixture was then added BnBr (166 g, 970.57 mmol, 2.99 equiv) dropwise with stirring at 0-10° C. The resulting solution was stirred overnight at room temperature. The reaction was then poured into water/ice (3 L). The resulting solution was extracted with ethyl acetate (2×1 L) and the organic layers combined. The resulting mixture was washed with water (5×1 L) and brine (2×1 L). The mixture was dried over anhydrous sodium sulfate and concentrated under vacuum. The resulting mixture was washed with PE/EA (50:1) to yield (4aR,6S,7R,8S,8aS)-7,8-bis(benzyloxy)-2-phenyl-6-(prop-2-en-1-yloxy)-hexahydro-2H-pyrano[3,2-d][1,3]dioxine as a white solid. ¹H NMR: (300 MHz, CDCl₃): δ 7.46-7.43 (m, 2H), 7.35-7.18 (m, 13H), 5.86-8.80 (m, 1H), 5.41 (s, 1H), 5.27-5.12 (m, 2H), 4.90 (s, 1H), 4.81-4.75 (m, 2H), 4.69-4.58 (m, 2H), 4.16-3.91 (m, 7H), 3.57 (s, 1H). LC-MS (ES, m/z): 506.2 [M+NH₄]⁺

### Step 3: Synthesis of (2R,3S,4S,5R,6S)-6-(allyloxy)-4,5-bis(benzyloxy)-2-((benzyloxy)methyl)tetrahydro-2H-pyran-3-ol

Into a 3-L 3-necked round-bottom flask purged and maintained with an inert atmosphere of nitrogen, was placed (4aR,6S,7R,8S,8aS)-7,8-bis(benzyloxy)-2-phenyl-6-(prop-2-en-1-yloxy)-hexahydro-2H-pyrano[3,2-d][1,3]dioxine (128.3 g, 262.60 mmol, 1.00 equiv), dry dichloromethane (1.8 L), 4 Å MS (120 g). This was followed by the addition of triethylsilane (91.5 g, 786.92 mmol, 3.00 equiv) at −78° C. To the resulting mixture was added trifluoromethanesulfonic acid (78.9 g, 525.73 mmol, 2.00 equiv) dropwise with stirring at −78° C. The resulting solution was stirred at −78° C. for 1 h. The reaction was then quenched by the addition of water (1.5 L). The resulting solution was extracted with DCM (2×1.5 L) and the organic layers combined. The resulting mixture was washed with saturated aqueous sodium bicarbonate (1×1.5 L) and brine (2×1.5 L). The mixture was dried over anhydrous sodium sulfate and concentrated under vacuum. The residue was applied onto a silica gel column with ethyl acetate/petroleum ether (0:1-1:3) to yield (2R,3S,4S,5R,6S)-4,5-bis(benzyloxy)-2-[(benzyloxy)methyl]-6-(prop-2-en-1-yloxy)oxan-3-ol as light yellow oil. LC-MS (ES, m/z): 508.2 [M+NH₄]⁺

### Step 4: Preparation of (2R,4R,5R,6S)-6-(allyloxy)-4,5-bis(benzyloxy)-2-((benzyloxy)methyl)dihydro-2H-pyran-3(4H)-one

Into a 3-L 3-necked round-bottom flask purged and maintained with an inert atmosphere of nitrogen, was placed (2R, 3S, 4S, 5R, 6S)-4, 5-bis(benzyloxy)-2-[(benzyloxy)methyl]-6-(prop-2-en-1-yloxy)oxan-3-ol (85 g, 173.26 mmol, 1.00 equiv), DMSO (850 mL). This was followed by the addition of Ac₂O (420 mL) dropwise with stirring at 10° C. The resulting solution was stirred for 1 h at 30° C. The resulting solution was allowed to react, with stirring, for an additional 1 h at 60° C. The reaction was then quenched by the addition of water/ice (1.5 L). The resulting solution was extracted with ethyl acetate (2×1 L) and the organic layers combined. The resulting mixture was washed with water (5×1 L) and saturated aqueous sodium bicarbonate (1×1 L). The resulting mixture was washed with brine (2×1 L). The mixture was dried over anhydrous sodium sulfate and concentrated under vacuum. The residue was applied onto a silica gel column with ethyl acetate/petroleum ether (0:1-1:4) to yield (2R,4R,5R,6S)-4,5-bis(benzyloxy)-2-[(benzyloxy)methyl]-6-(prop-2-en-1-yloxy)oxan-3-one as light yellow oil. LC-MS (ES, m/z): 506.2 [M+NH₄]⁺

### Step 5: Preparation of (2R,4R,5R,6S)-6-(allyloxy)-4,5-bis(benzyloxy)-2-((benzyloxy)methyl)-3,3-difluorotetrahydro-2H-pyran

Into a 1-L 3-necked round-bottom flask purged and maintained with an inert atmosphere of nitrogen, was placed (2R,4R,5R,6S)-4,5-bis(benzyloxy)-2-[(benzyloxy)methyl]-6-(prop-2-en-1-yloxy)oxan-3-one (50 g, 102.34 mmol, 1.00 equiv), dichloromethane (500 mL), DAST (66 g, 409.94 mmol, 4.01 equiv). The resulting solution was stirred overnight at room temperature. The reaction was then quenched by the addition of water/ice (1 L). The resulting solution was extracted with DCM (2×500 mL) and the organic layers combined. The resulting mixture was washed with saturated aqueous sodium bicarbonate (1×500 mL) and brine (2×500 mL). The mixture was dried over anhydrous sodium sulfate and concentrated under vacuum. The residue was applied onto a silica gel column with ethyl acetate/petroleum ether (0:1-1:20) to yield (2R,4R,5R,6S)-4,5-bis(benzyloxy)-2-[(benzyloxy)methyl]-3,3-difluoro-6-(prop-2-en-1-yloxy)oxane as colorless oil. LC-MS (ES, m/z): 508.2

### Step 6: Preparation of (3R,4R,6R)-3,4-bis(benzyloxy)-6-((benzyloxy)methyl)-5,5-difluorotetrahydro-2H-pyran-2-ol

Into a 1-L 3-necked round-bottom flask purged and maintained with an inert atmosphere of nitrogen, was placed (2R,4R,5R,6S)-4,5-bis(benzyloxy)-2-[(benzyloxy)methyl]-3,3-difluoro-6-(prop-2-en-1-yloxy)oxane (53 g, 103.81 mmol, 1.00 equiv), PMHS (101.6 g, 311.66 mmol, 3.00 equiv), ZnCl₂ (14.1 g, 103.44 mmol, 1.00 equiv), tetrakis(triphenylphosphane) palladium (12 g, 10.38 mmol, 0.10 equiv), THE (530 mL). The resulting solution was stirred overnight at room temperature. The reaction was then quenched by the addition of water (1 L). The resulting solution was extracted with ethyl acetate (2×500 mL) and the organic layers combined. The resulting mixture was washed with brine (2×500 mL). The mixture was dried over anhydrous sodium sulfate and concentrated under vacuum. The residue was applied onto a silica gel column with ethyl acetate/petroleum ether (0:1-1:5) to yield (3R,4R,6R)-3,4-bis(benzyloxy)-6-[(benzyloxy)methyl]-5,5-difluorooxan-2-ol as a white solid. LC-MS (ES, m/z): 488.2 [M+NH₄]⁺

### Step 7: Preparation of (3R,4R,6R)-3,4-bis(benzyloxy)-6-((benzyloxy)methyl)-5,5-difluorotetrahydro-2H-pyran-2-one

Into a 500-mL round-bottom flask purged and maintained with an inert atmosphere of nitrogen, was placed a solution of (2S, 3R, 4R, 6R)-3,4-bis(benzyloxy)-6-[(benzyloxy)methyl]-5,5-difluorooxan-2-ol (10 g, 21.25 mmol, 1.00 equiv) in DMSO (100 mL). This was followed by the addition of acetic anhydride (25 mL) at 0˜10° C. The resulting solution was stirred overnight at room temperature. The reaction was then quenched by the addition of water (100 mL). The resulting solution was extracted with ethyl acetate (3×100 mL) and the organic layers combined. The resulting mixture was washed with water (3×300 mL). The resulting mixture was washed with aqueous sodium bicarbonate (3×300 mL). The resulting mixture was concentrated under vacuum. The residue was applied onto a silica gel column with ethyl acetate/petroleum ether (1:5) to yield (3R,4R,6R)-3,4-bis(benzyloxy)-6-[(benzyloxy)methyl]-5,5-difluorooxan-2-one as a white solid.

### Example 1: Compound #25

### (2S,3R,4R,6R)-2-(5-(benzo[b]thiophen-2-ylmethyl)-2-hydroxy-4-methylphenyl)-5,5-difluoro-6-(hydroxymethyl)tetrahydro-2H-pyran-3,4-diol

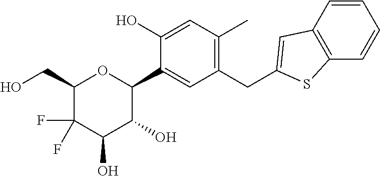 [SMILES: Cc1cc(O)c([C@@H]2O[C@H](CO)C(F)(F)[C@H](O)[C@H]2O)cc1Cc1cc2ccccc2s1]

To a mixture of 4-hydroxy-2-methylbenzaldehyde (2.72 g, 19.98 mmol, 1.00 equiv) in CH₃CN (30 mL) with K₂CO₃ (4.14 g, 29.95 mmol, 1.50 equiv) was added BnBr (3.8 g, 22.22 mmol, 1.11 equiv). The reaction mixture was stirred for 3 h at room temperature. Water was added and the mixture was extracted with EtOAc thrice. The mixture was then concentrated and the residue purified by chromatography on silica gel (10:1 PE/EA) to yield 4-(benzyloxy)-2-methylbenzaldehyde as white solid.

To a mixture of 4-(benzyloxy)-2-methylbenzaldehyde (2.26 g, 9.99 mmol, 1.00 equiv) in methanol (50 mL) was added pyridinium hydrobromide perbromide (90% purity, 3.52 g, 10 mmol, 1.00equiv) at 0° C. The reaction mixture was stirred for 12 h at room temperature. The mixture was concentrated and the resulting residue purified by on silica gel (10:1 PE/EA) to yield 4-(benzyloxy)-5-bromo-2-methylbenzaldehyde as a white solid.

To a mixture of 1-benzothiophene (900 mg, 6.71 mmol, 1.20 equiv) in tetrahydrofuran (20 mL) was added n-BuLi (2.5M in hexane, 2.7 mL, 1.2 equiv) dropwise with stirring at −78° C., the mixture was stirred for 20 mins at −78° C. After that, 4-(benzyloxy)-5-bromo-2-methylbenzaldehyde (1.7 g, 5.57 mmol, 1.00 equiv) in THE (5 mL) was added to the solution. The reaction mixture was stirred at −78° C. for 3h. NH₄Cl/H₂O was added and the mixture was extracted with EtOAc thrice. The combined extracts were washed with brine and dried over Na₂SO₄. The mixture was concentrated and the resulting residue purified by chromatography on silica gel (5:1 PE/EA) to yield benzo[b]thiophen-2-yl(4-(benzyloxy)-5-bromo-2-methylphenyl)methanol as a light yellow oil.

To a mixture of benzo[b]thiophen-2-yl(4-(benzyloxy)-5-bromo-2-methylphenyl)methanol (1 g, 2.28 mmol, 1.00 equiv) in dichloromethane (20 mL) with Et₃SiH (530 mg, 4.56 mmol, 2.00 equiv) was added CF₃CO₂H (520 mg, 4.56 mmol, 2.00 equiv) dropwise at 0° C. The reaction mixture was stirred for 4 h at 25° C. Sodium bicarbonate/H₂O was added and the mixture was extracted with DCM thrice. The combined extracts were washed with brine and dried over Na₂SO₄. The mixture was concentrated and the resulting residue purified by chromatography on silica gel (5:1 PE/EA) to yield 2-(4-(benzyloxy)-5-bromo-2-methylbenzyl)benzo[b]thiophene as a white solid.

To a mixture of 2-(4-(benzyloxy)-5-bromo-2-methylbenzyl)benzo[b]thiophene (3.47 g, 8.20 mmol, 0.91 equiv) in tetrahydrofuran (35 mL) was added n-butyllithium (3.3 mL, 1.00 equiv, 2.5 M/L) dropwise with stirring in 30 min. To this was added (3R,4S,6S)-3,4-bis(benzyloxy)-6-[(benzyloxy)methyl]-5-methylideneoxan-2-one (4 g, 9.00 mmol, 1.00 equiv). The resulting solution was stirred at −78° C. for 2 h in a dry ice bath. NH₄Cl/H₂O was added and the mixture was extracted with EtOAc thrice. The combined extracts were washed with brine and dried over Na₂SO₄. The mixture was concentrated and the resulting residue purified by chromatography on silica gel (3:1 PE/EA) to yield (3R,4S,6S)-2-(5-(benzo[b]thiophen-2-ylmethyl)-2-(benzyloxy)-4-methylphenyl)-3,4-bis(benzyloxy)-6-((benzyloxy)methyl)-5-methylenetetrahydro-2H-pyran-2-ol as a yellow oil.

To a mixture of (3R,4S,6S)-2-(5-(benzo[b]thiophen-2-ylmethyl)-2-(benzyloxy)-4-methylphenyl)-3,4-bis(benzyloxy)-6-((benzyloxy)methyl)-5-methylenetetrahydro-2H-pyran-2-ol (5 g, 6.34 mmol, 1.00 equiv) in dichloromethane (50 mL) was added BF₃.Et₂O (1.8 g, 12.68 mmol, 2.00 equiv), triethylsilane (2.2 g, 18.92 mmol, 3.00 equiv). The resulting solution was stirred for 1 h at 0° C. in a water/ice bath. Sodium bicarbonate/H₂O was added and the mixture was extracted with DCM thrice. The combined extracts were washed with brine and dried over Na₂SO₄. The mixture was concentrated and the resulting residue purified by chromatography on silica gel (5:1 PE/EA) to yield (2S,3R,4S,6S)-2-(5-(benzo[b]thiophen-2-ylmethyl)-2-(benzyloxy)-4-methylphenyl)-3,4-bis(benzyloxy)-6-((benzyloxy)methyl)-5-methylenetetrahydro-2H-pyran as a yellow oil.

To a mixture of (2S,3R,4S,6S)-2-(5-(benzo[b]thiophen-2-ylmethyl)-2-(benzyloxy)-4-methylphenyl)-3,4-bis(benzyloxy)-6-((benzyloxy)methyl)-5-methylenetetrahydro-2H-pyran (4.8 g, 6.21 mmol, 1.00 equiv) in acetone/H₂O (50/6 mL), was added OsO₄ (31 mg, 0.12 mmol, 0.02 equiv), NMO (2.17 g, 18.55 mmol, 3.00 equiv). The resulting solution was stirred for 2 h at 45° C. in an oil bath. NH₄Cl/H₂O was added and the mixture was extracted with EtOAc thrice. The combined extracts were washed with brine and dried over Na₂SO₄. The mixture was concentrated and the resulting residue purified by chromatography on silica gel (3:1 PE/EA) to yield (2R,4R,5S,6S)-6-(5-(benzo[b]thiophen-2-ylmethyl)-2-(benzyloxy)-4-methylphenyl)-4,5-bis(benzyloxy)-2-((benzyloxy)methyl)-3-(hydroxymethyl)tetrahydro-2H-pyran-3-ol as a yellow oil.

To a mixture of (2R,4R,5S,6S)-6-(5-(benzo[b]thiophen-2-ylmethyl)-2-(benzyloxy)-4-methylphenyl)-4,5-bis(benzyloxy)-2-((benzyloxy)methyl)-3-(hydroxymethyl)tetrahydro-2H-pyran-3-ol (3 g, 3.72 mmol, 1.00 equiv) in dichloromethane (30 mL) was added PhI(OAc)₂ (2.39 g, 7.42 mmol, 2.00 equiv). The resulting solution was stirred overnight at room temperature. NH₄Cl/H₂O was added and the mixture was extracted with EtOAc thrice. The combined extracts were washed with brine and dried over Na₂SO₄. The mixture was concentrated and the resulting residue purified by chromatography on silica gel (4:1 PE/EA) to yield (2R,4R,5S,6S)-6-(5-(benzo[b]thiophen-2-ylmethyl)-2-(benzyloxy)-4-methylphenyl)-4,5-bis(benzyloxy)-2-((benzyloxy)methyl)dihydro-2H-pyran-3(4H)-one as a yellow oil.

To a mixture of (2R,4R,5S,6S)-6-(5-(benzo[b]thiophen-2-ylmethyl)-2-(benzyloxy)-4-methylphenyl)-4,5-bis(benzyloxy)-2-((benzyloxy)methyl)dihydro-2H-pyran-3(4H)-one (600 mg, 0.77 mmol, 1.00 equiv) in dichloromethane (10 mL) was added DAST (623 mg, 3.87 mmol, 5.00 equiv). The resulting solution was stirred overnight at room temperature. NH₄Cl/H₂O was added and the mixture was extracted with EtOAc thrice. The combined extracts were washed with brine and dried over Na₂SO₄. The mixture was concentrated and the resulting residue purified by chromatography on silica gel (4:1 PE/EA) to yield (2R,4R,5S,6S)-6-(5-(benzo[b]thiophen-2-ylmethyl)-2-(benzyloxy)-4-methylphenyl)-4,5-bis(benzyloxy)-2-((benzyloxy)methyl)-3,3-difluorotetrahydro-2H-pyran as a yellow oil.

To a mixture of (2R,4R,5S,6S)-6-(5-(benzo[b]thiophen-2-ylmethyl)-2-(benzyloxy)-4-methylphenyl)-4,5-bis(benzyloxy)-2-((benzyloxy)methyl)-3,3-difluorotetrahydro-2H-pyran (1 g, 1.25 mmol, 1.00 equiv) in dichloromethane (20 mL) was added 1,2,3,4,5-pentamethylbenzene (1.86 g, 12.55 mmol, 10.00 equiv). This was followed by the addition of trichloroborane (15 mL, 10.00 equiv) dropwise with stirring at −78° C. The resulting solution was stirred at −78° C. for 1 h in a dry ice bath. Methanol (5 mL) was then added. The mixture was concentrated and the resulting residue purified by chromatography on a C18 reversed phase column to yield (2S,3R,4R,6R)-2-(5-(benzo[b]thiophen-2-ylmethyl)-2-hydroxy-4-methylphenyl)-5,5-difluoro-6-(hydroxymethyl)tetrahydro-2H-pyran-3,4-diol, the title compound, as a white solid.

¹H-NMR (300 MHz, CD₃OD) δ: 7.74 (d, J=7.8 Hz, 1H), 7.64 (d, J=7.5 Hz, 1H), 7.21-7.31 (m, 3H), 6.92 (s, 1H), 6.71 (s, 1H), 4.75 (d, J=9.2 Hz, 1H), 4.18 (d, J=1.2 Hz, 2H), 3.74-3.97 (m, 5H), 2.25 (s, 3H). MS(ES) m/z: 454.0 [M+NH₄]⁺

### Example 2: Compound #60

### (2S,3R,4R,6R)-2-(5-(benzo[b]thiophen-2-ylmethyl)-2-hydroxy-3,4-dimethylphenyl)-5,5-difluoro-6-(hydroxymethyl)tetrahydro-2H-pyran-3,4-diol

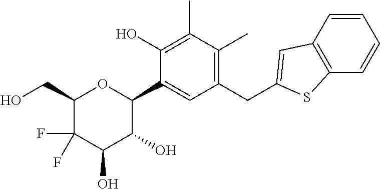 [SMILES: Cc1c(Cc2cc3ccccc3s2)cc([C@@H]2O[C@H](CO)C(F)(F)[C@H](O)[C@H]2O)c(O)c1C]

To a mixture of 4-methoxy-2,3-dimethylbenzaldehyde (3.2 g, 19.49 mmol, 1.00 equiv) in DCM (80 ml) was added dropwise tribromoborane (10 g, 39.92 mmol, 2.05 equiv) at −78° C. The reaction mixture was stirred at room temperature for 3 h. Water was added and the mixture was extracted with DCM thrice. The combined extracts were washed with water, saturated brine and dried over anhydrous Na₂SO₄. The mixture was concentrated and the resulting residue purified by chromatography on silica gel (1:5 EA/PE) to yield 4-hydroxy-2,3-dimethylbenzaldehyde as a white solid.

To a mixture of 4-hydroxy-2,3-dimethylbenzaldehyde (1.4 g, 9.32 mmol, 1.00 equiv) in MeOH (20 ml) was added pyridinium hydrobromide perbromide (3.0 g, 9.38 mmol, 1.01 equiv) at room temperature. The reaction mixture was stirred at room temperature for 5 h. Water was added and the mixture was extracted with EA thrice. The combined extracts were washed with water, saturated brine and dried over anhydrous Na₂SO₄. The mixture was concentrated and the resulting residue purified by chromatography on silica gel (1:4 EA/PE) to yield 5-bromo-4-hydroxy-2,3-dimethylbenzaldehyde as a white solid.

To a mixture of 5-bromo-4-hydroxy-2,3-dimethylbenzaldehyde (1.4 g, 6.11 mmol, 1.00 equiv) in DMF (15 ml) with potassium carbonate (1.3 g, 9.41 mmol, 1.54 equiv) was added BnBr (1.26 g, 7.37 mmol, 1.21 equiv) at 0° C. The reaction mixture was stirred at room temperature for 3 h. Water was added and the mixture was extracted with EA thrice. The combined extracts were washed with water, saturated brine and dried over anhydrous Na₂SO₄. The mixture was concentrated and the resulting residue purified by chromatography on silica gel (1:4 EA/PE) to yield 4-(benzyloxy)-5-bromo-2,3-dimethylbenzaldehyde as a white solid.

To a mixture of 1-benzothiophene (510 mg, 3.80 mmol, 1.21 equiv) in THE (15 ml) was added dropwise n-BuLi (2.5 M in n-hexane) (1.6 ml, 4 mmol, 1.27 equiv) at −78° C. under N₂. The reaction mixture was stirred at −78° C. for 30 min. 4-(benzyloxy)-5-bromo-2,3-dimethylbenzaldehyde (1.0 g, 3.13 mmol, 1.00 equiv) in THE (5 ml) was added dropwise at −78° C. The reaction mixture was stirred at −78° C. for 1 h. Saturated NH₄Cl (aq) was added and the mixture was extracted with EA thrice. The combined extracts were washed with water, saturated brine and dried over anhydrous Na₂SO₄. The mixture was concentrated and the resulting residue purified by chromatography on silica gel (1:4 EA/PE) to yield benzo[b]thiophen-2-yl(4-(benzyloxy)-5-bromo-2,3-dimethylphenyl)methanol as a white solid.

To a solution of benzo[b]thiophen-2-yl(4-(benzyloxy)-5-bromo-2,3-dimethylphenyl)methanol (1.4 g, 3.09 mmol, 1.00 equiv) in DCM (15 ml) with Et₃SiH (610 mg, 5.25 mmol, 1.70 equiv) was added dropwise TFA (640 mg, 5.66 mmol, 1.83 equiv) at 0° C. The reaction mixture was stirred at 0° C. for 1 h. Saturated NaHCO₃ (aq) was added and the mixture was extracted with DCM thrice. The combined extracts were washed with water, saturated brine and dried over anhydrous Na₂SO₄. The mixture was concentrated and the resulting residue purified by chromatography on silica gel (1:5 EA/PE) to yield 2-(4-(benzyloxy)-5-bromo-2,3-dimethylbenzyl)benzo[b]thiophene as a yellow oil.

To a solution of 2-(4-(benzyloxy)-5-bromo-2,3-dimethylbenzyl)benzo[b]thiophene (223 mg, 0.51 mmol, 1.00 equiv) in THE (3 ml) was added dropwise n-BuLi (2.5 M in n-hexane) (0.21 ml, 0.52 mmol, 1.03 equiv) at −78° C. under N₂. The reaction mixture was stirred at −78° C. for 30 min. (3R,4R,6R)-3,4-bis(benzyloxy)-6-[(benzyloxy)methyl]-5,5-difluorooxan-2-one (200 mg, 0.43 mmol, 0.84 equiv) in THE (0.5 ml) was added dropwise at −78° C. The reaction mixture was stirred at −78° C. for 2 h. Saturated NH₄Cl (aq) was added and the mixture was extracted with EA thrice. The combined extracts were washed with water, saturated brine and dried over anhydrous Na₂SO₄. The mixture was concentrated and the resulting residue purified by chromatography on silica gel (1:4 EA/PE) to yield (3R,4R,6R)-2-(5-(benzo[b]thiophen-2-ylmethyl)-2-(benzyloxy)-3,4-dimethylphenyl)-3,4-bis(benzyloxy)-6-((benzyloxy)methyl)-5,5-difluorotetrahydro-2H-pyran-2-ol as a yellow oil. MS (ES) m/z: 809 [M-OH]⁺.

To a solution of (3R,4R,6R)-2-(5-(benzo[b]thiophen-2-ylmethyl)-2-(benzyloxy)-3,4-dimethylphenyl)-3,4-bis(benzyloxy)-6-((benzyloxy)methyl)-5,5-difluorotetrahydro-2H-pyran-2-ol (180 mg, 0.22 mmol, 1.00 equiv) in DCM (3 ml) with Et₃SiH (50 mg, 0.43 mmol, 1.98 equiv) was added BF₃.Et₂O (46 mg, 0.43 mmol, 1.98 equiv) at 0° C. The reaction mixture was stirred at 0° C. for 1 h. Saturated NaHCO₃ (aq) was added and the mixture was extracted with DCM thrice. The combined extracts were washed with water, saturated brine and dried over anhydrous Na₂SO₄. The mixture was concentrated and the resulting residue purified by chromatography on silica gel (1:4 EA/PE) to yield (2R,4R,5S,6S)-6-(5-(benzo[b]thiophen-2-ylmethyl)-2-(benzyloxy)-3,4-dimethylphenyl)-4,5-bis(benzyloxy)-2-((benzyloxy)methyl)-3,3-difluorotetrahydro-2H-pyran as a colorless oil. MS (ES) m/z: 828 [M+NH₄]⁺.

To a solution of (2R,4R,5S,6S)-6-(5-(benzo[b]thiophen-2-ylmethyl)-2-(benzyloxy)-3,4-dimethylphenyl)-4,5-bis(benzyloxy)-2-((benzyloxy)methyl)-3,3-difluorotetrahydro-2H-pyran (81 mg, 0.10 mmol, 1.00 equiv) with 1,2,3,4,5-pentamethylbenzene (160 mg, 1.08 mmol, 10.81 equiv) in DCM (5 ml) was added BCl₃ (1 M in DCM) (1.6 ml, 1.6 mmol, 16 equiv) at −78° C. The reaction mixture was stirred at −78° C. for 30 min. The mixture was concentrated and the resulting residue purified by chromatography on C18 reverse column (0-70% MeCN/H₂O) to yield (2S,3R,4R,6R)-2-(5-(benzo[b]thiophen-2-ylmethyl)-2-hydroxy-3,4-dimethylphenyl)-5,5-difluoro-6-(hydroxymethyl)tetrahydro-2H-pyran-3,4-diol, the title compound, as a white solid.

¹H-NMR (300 MHz, CD₃OD) δ: 7.18 (d, J=7.8 Hz, 1H), 7.60 (d, J=7.8 Hz, 1H), 7.20-7.29 (m, 2H), 7.13 (s, 1H), 6.86 (s, 1H), 4.64 (d, J=9.6 Hz, 1H), 4.20 (s, 2H), 3.70-3.91 (m, 5H), 2.19 (s, 6H); MS (ES) m/z: 468 [M+NH₄]⁺.

Additional representative compounds of the present invention, detailed in Table 3, below, were similarly prepared according to the procedures described in Examples 1 and 2 above, selecting and substituting suitably substituted reagents and starting materials, as would be readily recognized by those skilled in the art.

TABLE 3
Representative Compounds of Formula (I)

| ID No. | Compound name/Analytical data |
|---|---|
| 27 | (2S,3R,4R,6R)-2-[5-(benzothiophen-2-ylmethyl)-4-chloro-2-hydroxy- |
| | phenyl]-5,5-difluoro-6-(hydroxymethyl)tetrahydropyran-3,4-diol |
| | ¹H NMR (METHANOL-d₄) δ: 7.72 (d, J = 8.1 Hz, 1H), 7.63 (d, J = 7.6 Hz, |
| | 1H), 7.37 (s, 1H), 7.18-7.30 (m, 2H), 6.98 (s, 1H), 6.89 (s, 1H), 4.72 (d, |
| | J = 9.6 Hz, 1H), 4.20-4.32 (m, 2H), 3.69-3.91 (m, 5H). MS(ESI⁺, m/z): |
| | [M + Na]⁺ m/z 479.0 |
| 26 | (2S,3R,4R,6R)-2-[5-(benzothiophen-2-ylmethyl)-4-ethyl-2-hydroxy- |
| | phenyl]-5,5-difluoro-6-(hydroxymethyl)tetrahydropyran-3,4-diol |
| | ¹H NMR (METHANOL-d₄) δ: 7.69 (br d, J = 7.6 Hz, 1H), 7.59 (d, J = 7.6 |
| | Hz, 1H), 7.17-7.28 (m, 3H), 6.88 (s, 1H), 6.73 (s, 1H), 4.73 (br d, J = 9.1 |
| | Hz, 1H), 4.14 (s, 2H), 3.74-3.94 (m, 5H), 2.58 (q, J = 7.6 Hz, 2H), 1.10 |
| | (t, J = 7.6 Hz, 3H). MS(ESI⁺, m/z): [M + Na]⁺ m/z 473.10 |
| 36 | (2S,3R,4R,6R)-2-[5-(benzothiophen-2-ylmethyl)-4-chloro-2-methoxy- |
| | phenyl]-5,5-difluoro-6-(hydroxymethyl)tetrahydropyran-3,4-diol |
| | ¹H NMR (ACETONITRILE-d₃) δ: 7.81 (s, 1H), 7.73 (d, J = 7.6 Hz, 1H), |
| | 7.47 (s, 1H), 7.26-7.38 (m, 2H), 7.09-7.14 (m, 2H), 4.75 (d, J = 10.1 Hz, |
| | 1H), 4.34 (s, 2H), 3.78-3.89 (m, 5H), 3.59-3.77 (m, 3H). MS(ESI⁺, |
| | m/z): [M + Na]⁺ m/z 479.10 |
| 35 | (2S,3R,4R,6R)-2-[5-(benzothiophen-2-ylmethyl)-2-methoxy-4-methyl- |
| | phenyl]-5,5-difluoro-6-(hydroxymethyl)tetrahydropyran-3,4-diol |
| | ¹H NMR (ACETONITRILE-d₃) δ: 7.81 (d, J = 8.1 Hz, 1H), 7.71 (d, J = 8.1 |
| | Hz, 1H), 7.24-7.38 (m, 3H), 7.02 (s, 1H), 6.89 (s, 1H), 4.75 (d, J = 9.1 |
| | Hz, 1H), 4.23 (s, 2H), 3.71-3.90 (m, 7H), 3.59-3.69 (m, 1H), 2.33 (s, |
| | 3H). MS(ESI⁺, m/z): [M + Na]⁺ m/z 459.00 |
| 40 | 2-[4-(benzothiophen-2-ylmethyl)-2-[(2S,3R,4R,6R)-5,5-difluoro-3,4- |
| | dihydroxy-6-(hydroxymethyl)tetrahydropyran-2-yl]-5-methyl- |
| | phenoxy]acetonitrile |
| | ¹H NMR (METHANOL-d₄) δ: 7.72 (d, J = 8.1 Hz, 1H), 7.62 (d, J = 8.1 Hz, |
| | 1H), 7.37 (s, 1H), 7.18-7.30 (m, 2H), 6.99 (s, 1H), 6.91 (s, 1H), 4.97 (s, |
| | 2H), 4.71 (d, J = 9.6 Hz, 1H), 4.22 (s, 2H), 3.71-3.93 (m, 5H), 2.33 (s, |
| | 3H). MS(ESI⁺, m/z): [M + Na]⁺ m/z 498.15 |

### Example 3: Compound #44

### (2S,3R,4R,6R)-2-(5-(chroman-6-ylmethyl)-2-hydroxy-4-methoxyphenyl)-5,5-difluoro-6-(hydroxymethyl)tetrahydro-2H-pyran-3,4-diol

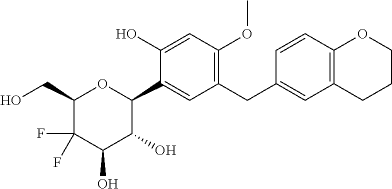 [SMILES: COc1cc(O)c([C@@H]2O[C@H](CO)C(F)(F)[C@H](O)[C@H]2O)cc1Cc1ccc2c(c1)CCCO2]

To a mixture of 6-bromochromane (2.0 g, 9.43 mmol, 1.00 equiv) in THE (20 mL) was added n-BuLi (2.5 M in hexane, 3.76 mL, 1.00 equiv) dropwise at −78° C. The reaction mixture was stirred at −78° C. for 30 min. To the mixture was then added a solution of 4-(benzyloxy)-5-bromo-2-methoxybenzaldehyde (2.74 g, 8.56 mmol, 0.91 equiv) in THE (2 mL) dropwise at −78° C. The reaction mixture was stirred for 2 h at −78° C. NH₄Cl/H₂O was added and the mixture was extracted with EtOAc thrice. The combined extracts were washed with brine and dried over Na₂SO₄. The mixture was concentrated and the resulting residue purified by chromatography on silica gel (3:1 PE/EA) to yield (4-(benzyloxy)-5-bromo-2-methoxyphenyl)(chroman-6-yl)methanol as a colorless oil.

To a mixture of (4-(benzyloxy)-5-bromo-2-methoxyphenyl)(chroman-6-yl)methanol (3.5 g, 7.69 mmol, 1.00 equiv) in dichloromethane (35 mL) with Et₃SiH (2.67 g, 22.96 mmol, 2.99 equiv) was added CF₃COOH (1.75 g, 15.35 mmol, 2.00 equiv) at 0° C. The reaction mixture was stirred for 2 h at 0° C. Sodium bicarbonate was added and the mixture was extracted with DCM thrice. The combined extracts were washed with brine and dried over Na₂SO₄. The mixture was concentrated and the resulting residue purified by chromatography on silica gel (4:1 PE/EA) to yield 6-(4-(benzyloxy)-5-bromo-2-methoxybenzyl)chromane as a colorless oil. MS (ES) m/z: 456, 458[M+NH₄]⁺.

To a mixture of 6-(4-(benzyloxy)-5-bromo-2-methoxybenzyl)chromane (309 mg, 0.703 mmol, 1.00 equiv) in tetrahydrofuran (4 mL) was added n-BuLi (2.5 M in hexane, 0.282 mL, 1.00 equiv) dropwise at −78° C., and The reaction mixture was stirred at −78° C. for 30 min. To this was added a solution of (3R,4R,6R)-3,4-bis(benzyloxy)-6-[(benzyloxy)methyl]-5,5-difluorooxan-2-one (300 mg, 0.64 mmol, 0.91 equiv) in tetrahydrofuran (2 mL) dropwise at −78° C. The reaction mixture was stirred for 2 h at −78° C. NH₄Cl/H₂O was added and the mixture was extracted with EtOAc thrice. The combined extracts were washed with brine and dried over Na₂SO₄. The mixture was concentrated and the resulting residue purified by chromatography on silica gel (3:1 PE/EA) to yield (3R,4R,6R)-3,4-bis(benzyloxy)-2-(2-(benzyloxy)-5-(chroman-6-ylmethyl)-4-methoxyphenyl)-6-((benzyloxy)methyl)-5,5-difluorotetrahydro-2H-pyran-2-ol as a colorless oil. MS (ES) m/z: 846 [M+NH₄]⁺

To a mixture of (3R,4R,6R)-3,4-bis(benzyloxy)-2-(2-(benzyloxy)-5-(chroman-6-ylmethyl)-4-methoxyphenyl)-6-((benzyloxy)methyl)-5,5-difluorotetrahydro-2H-pyran-2-ol (300 mg, 0.36 mmol, 1.00 equiv) in dichloromethane (5 mL) with Et₃SiH (84 mg, 0.72 mmol, 2.00 equiv) was added BF₃.Et₂O (77 mg, 0.54 mmol, 1.50 equiv) dropwise at 0° C. The reaction mixture was stirred for 2 h at 0° C. Sodium bicarbonate was added and the mixture was extracted with DCM twice. The combined extracts were washed with brine and dried over Na₂SO₄. The mixture was concentrated and the resulting residue purified by chromatography on silica gel (3:1 PE/EA) to yield 6-(4-(benzyloxy)-5-((2S,3S,4R,6R)-3,4-bis(benzyloxy)-6-((benzyloxy)methyl)-5,5-difluorotetrahydro-2H-pyran-2-yl)-2-methoxybenzyl)chromane as a colorless oil. MS (ES) m/z: 830 [M+NH₄]⁺

To a mixture of 6-(4-(benzyloxy)-5-((2S,3S,4R,6R)-3,4-bis(benzyloxy)-6-((benzyloxy)methyl)-5,5-difluorotetrahydro-2H-pyran-2-yl)-2-methoxybenzyl)chromane (500 mg, 0.62 mmol, 1.00 equiv) in methanol/EtOAc (5 mL/5 mL) was added Pd(OH)₂/C (500 mg) at room temperature, H₂ was introduced into. The reaction mixture was stirred overnight at room temperature. The solid was filtered out. The mixture was concentrated and the resulting residue purified by chromatography on C18 (10%-40% CH₃CN/H₂O) to yield (2S,3R,4R,6R)-2-(5-(chroman-6-ylmethyl)-2-hydroxy-4-methoxyphenyl)-5,5-difluoro-6-(hydroxymethyl)tetrahydro-2H-pyran-3,4-diol, the title compound, as a white solid.

¹H-NMR: (300 MHz, CD₃OD) δ: 7.04 (s, 1H), 6.86-6.82 (m, 2H), 6.56 (d, J=8.1 Hz, 1H), 6.44 (s, 1H), 4.65 (d, J=9.0 Hz, 1H), 4.10 (t, J=5.1 Hz, 2H), 3.67-3.90 (m, 10H), 2.71 (t, J=6.6 Hz, 2H), 1.93-1.98 (m, 2H). MS (ES) m/z: 470 [M+NH₄]⁺.

### Example 4: Compound #56

### (2S,3R,4R,6R)-2-(5-((2,3-dihydrobenzo[b][1,4]dioxin-6-yl)methyl)-2-hydroxy-4-methylphenyl)-5,5-difluoro-6-(hydroxymethyl)tetrahydro-2H-pyran-3,4-diol

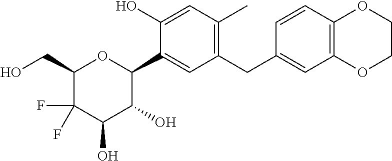 [SMILES: Cc1cc(O)c([C@@H]2O[C@H](CO)C(F)(F)[C@H](O)[C@H]2O)cc1Cc1ccc2c(c1)OCCO2]

To a mixture of 6-bromo-2,3-dihydro-1,4-benzodioxine (679 mg, 3.15 mmol, 1.2 equiv) in THE (15 ml) was added n-BuLi (2.5 M in n-hexane) (1.26 ml, 3.15 mmol, 1.2 equiv) dropwise at −78° C. under N₂. The reaction mixture was stirred at −78° C. for 30 min. 4-(Cenzyloxy)-5-bromo-2-methylbenzaldehyde (800 mg, 2.63 mmol, 1.00 equiv) in THE (15 ml) was added dropwise at −78° C. The reaction mixture was stirred at −78° C. for 1 h. Saturated NH₄Cl (aq) was added and the mixture was extracted with EA thrice. The combined extracts were washed with water, saturated brine and dried over anhydrous Na₂SO₄. The mixture was concentrated and the resulting residue purified by chromatography on silica gel (1:2 EA/PE) to yield (4-(benzyloxy)-5-bromo-2-methylphenyl)(2,3-dihydrobenzo[b][1,4]dioxin-6-yl)methanol as a yellow oil.

To a mixture of (4-(benzyloxy)-5-bromo-2-methylphenyl)(2,3-dihydrobenzo[b][1,4]dioxin-6-yl)methanol (1.9 g, 4.31 mmol, 1.00 equiv) in DCM (20 ml) with Et₃SiH (788 mg, 6.79 mmol, 1.57 equiv) was added BF₃.Et₂O (962 mg, 6.78 mmol, 1.57 equiv) at 0° C. The reaction mixture was stirred at 0° C. for 1h. Saturated NaHCO₃ (aq) was added and the mixture was extracted with DCM thrice. The combined extracts were washed with water, saturated brine and dried over anhydrous Na₂SO₄. The mixture was concentrated and the resulting residue purified by chromatography on silica gel (1:3 EA/PE) to yield 6-(4-(benzyloxy)-5-bromo-2-methylbenzyl)-2,3-dihydrobenzo[b][1,4]dioxine as a yellow oil. ¹H NMR (400 MHz, CD₃OD) δ 7.25-7.49 (m, 6H), 6.78-6.72 (m, 2H), 6.58-6.55 (m, 2H), 5.12 (s, 2H), 6.22 (s, 4H), 3.77 (s, 2H), 2.17 (s, 3H).

To a mixture of 6-(4-(benzyloxy)-5-bromo-2-methylbenzyl)-2,3-dihydrobenzo[b][1,4]dioxine (109.6 mg, 0.26 mmol, 1.00 equiv) in THE (3 ml) was added dropwise n-BuLi (2.5 M in n-hexane) (0.11 mL, 0.26 mmol, 1.0 equiv) at −78° C. under N₂. The reaction mixture was stirred at −78° C. for 30 min. (3R,4R,6R)-3,4-bis(benzyloxy)-6-[(benzyloxy)methyl]-5,5-difluorooxan-2-one (110 mg, 0.23 mmol, 0.91 equiv) in THE (0.5 ml) was added dropwise at −78° C. The reaction mixture was stirred at −78° C. for 1h. Saturated NH₄Cl (aq) was added and the mixture was extracted with EA thrice. The combined extracts were washed with water, saturated brine and dried over anhydrous Na₂SO₄. The mixture was concentrated and the resulting residue purified by chromatography on silica gel (1:3 EA/PE) to yield (3R,4R,6R)-3,4-bis(benzyloxy)-2-(2-(benzyloxy)-5-((2,3-dihydrobenzo[b][1,4]dioxin-6-yl)methyl)-4-methylphenyl)-6-((benzyloxy)methyl)-5,5-difluorotetrahydro-2H-pyran-2-ol as a yellow oil. MS (ES) m/z: 797 [M-OH]⁺.

To a mixture of (3R,4R,6R)-3,4-bis(benzyloxy)-2-(2-(benzyloxy)-5-((2,3-dihydrobenzo[b][1,4]dioxin-6-yl)methyl)-4-methylphenyl)-6-((benzyloxy)methyl)-5,5-difluorotetrahydro-2H-pyran-2-ol (58 mg, 0.07 mmol, 1.00 equiv) in DCM (2 ml) with Et₃SiH (17 mg, 0.15 mmol, 2.05 equiv) was added BF₃.Et₂O (20 mg, 0.14 mmol, 2.05 equiv) at 0° C. The reaction mixture was stirred at 0° C. for 1h. Saturated NaHCO₃ (aq) was added and the mixture was extracted with DCM thrice. The combined extracts were washed with water, saturated brine and dried over anhydrous Na₂SO₄. The mixture was concentrated and the resulting residue purified by chromatography on silica gel (1:3 EA/PE) to yield 6-(4-(benzyloxy)-5-((2S,3S,4R,6R)-3,4-bis(benzyloxy)-6-((benzyloxy)methyl)-5,5-difluorotetrahydro-2H-pyran-2-yl)-2-methylbenzyl)-2,3-dihydrobenzo[b][1,4]dioxine as a colorless oil. MS (ES) m/z: 816 [M+NH₄]⁺.

To a mixture of 6-(4-(benzyloxy)-5-((2S,3S,4R,6R)-3,4-bis(benzyloxy)-6-((benzyloxy)methyl)-5,5-difluorotetrahydro-2H-pyran-2-yl)-2-methylbenzyl)-2,3-dihydrobenzo[b][1,4]dioxine (50 mg, 0.06 mmol, 1.00 equiv) in MeOH (5 ml) was added Pd/C (10% in weight) (20 mg) at room temperature, H₂ was introduced into. The reaction mixture was stirred at room temperature for 16 h. The mixture was filtered, the filtrated was concentrated and the resulting residue purified by chromatography on C18 reverse column (0-70% MeCN/H₂O) to yield (2S,3R,4R,6R)-2-(5-((2,3-dihydrobenzo[b][1,4]dioxin-6-yl)methyl)-2-hydroxy-4-methylphenyl)-5,5-difluoro-6-(hydroxymethyl)tetrahydro-2H-pyran-3,4-diol, the title compound, as a white solid.

¹H NMR (300 MHz, CD₃OD) δ: 6.99 (s, 1H), 6.51-6.59 (m, 2H), 6.42-6.47 (m, 2H), 4.59 (d, J=9.2 Hz, 1H), 4.07 (s, 4H), 3.63-3.82 (m, 7H), 1.99 (s, 3H); MS (ES) m/z: 438 [M+NH₄]⁺.

### Example 5: Compound #111

### (2S,3R,4R,6R)-2-(4-chloro-5-((2,3-dihydrobenzofuran-6-yl)methyl)-2-hydroxyphenyl)-5,5-difluoro-6-(hydroxymethyl)-tetrahydro-2H-pyran-3,4-diol

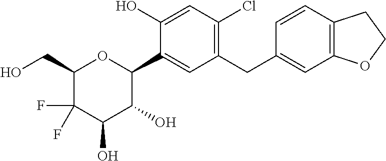 [SMILES: OC[C@H]1O[C@@H](c2cc(Cc3ccc4c(c3)OCC4)c(Cl)cc2O)[C@H](O)[C@@H](O)C1(F)F]

To a mixture of 1,2-dibromoethane (10.8 g, 58.02 mmol, 1.46 equiv) and K₂CO₃ (21.4 g, 155.07 mmol, 3.90 equiv) in acetonitril (150 mL) was added 2,5-dibromophenol (10 g, 40.0 mmol, 1.00 equiv) at room temperature. The reaction mixture was stirred for 4 h at 80° C. Water was added and the mixture was extracted with EtOAc thrice. The combined extracts were washed with brine and dried over Na₂SO₄. The mixture was concentrated and the resulting residue purified by chromatography on silica gel (4:1 PE/EA) to yield 1,4-dibromo-2-(2-bromoethoxy)benzene as a colorless oil. ¹H NMR (400 MHz, Chloroform-d) δ: 7.43 (d, J=8.9 Hz, 1H), 7.08-6.98 (m, 2H), 4.35 (t, J=6.4 Hz, 2H), 3.70 (t, J=6.4 Hz, 2H).

To a mixture of 1,4-dibromo-2-(2-bromoethoxy)benzene (8 g, 22.29 mmol, 1.00 equiv) in THE (50 mL) was added n-BuLi (10.78 mL, 2.5 M/L, 1.2 equiv) dropwise at −100° C. The reaction mixture was stirred at −100° C. for 3 h, NH₄Cl/H₂O was added and the mixture was extracted with EtOAc thrice. The combined extracts were washed with brine and dried over Na₂SO₄. The mixture was concentrated and the resulting residue purified by chromatography on silica gel (3:1 PE/EA) to yield 6-bromo-2,3-dihydrobenzofuran as a colorless oil. ¹H NMR (300 MHz, Chloroform-d) δ: 7.05 (m, 1H), 7.01-6.93 (m, 2H), 4.60 (t, J=8.7 Hz, 2H), 3.30-3.10 (m, 2H).

To a mixture of 6-bromo-2,3-dihydrobenzofuran (2 g, 10.05 mmol, 1.00 equiv) in tetrahydrofuran (25 mL) was added n-BuLi (4.04 mL, 2.5M/L) dropwise at −78° C. 30 min later, to this was added a solution of 4-(benzyloxy)-5-bromo-2-chlorobenzaldehyde (2.9 g, 8.91 mmol, 0.89 equiv) in tetrahydrofuran (2 mL). The reaction mixture was stirred at −78° C. for 2 h, NH₄Cl/H₂O was added and the mixture was extracted with EtOAc thrice. The combined extracts were washed with brine and dried over Na₂SO₄. The mixture was concentrated and the resulting residue purified by chromatography on silica gel (3:1 PE/EA) to yield (4-(benzyloxy)-5-bromo-2-chlorophenyl)(2,3-dihydrobenzofuran-6-yl)methanol as a colorless oil.

To a mixture of (4-(benzyloxy)-5-bromo-2-chlorophenyl)(2,3-dihydrobenzofuran-6-yl)methanol (4.08 g, 9.15 mmol, 1.00 equiv) in dichloromethane (40 mL) with Et₃SiH (2.14 g, 18.40 mmol, 2.01 equiv) was added trifluoroacetic acid (1.57 g, 13.77 mmol, 1.5 equiv) dropwise at 0° C. The reaction mixture was stirred for 1 h at 0° C., NaHCO₃/H₂O was added and the mixture was extracted with EtOAc thrice. The combined extracts were washed with brine and dried over Na₂SO₄. The mixture was concentrated and the resulting residue purified by chromatography on silica gel (4:1 PE/EA) to yield 6-(4-(benzyloxy)-5-bromo-2-chlorobenzyl)-2,3-dihydrobenzofuran as a white solid. MS (ES) m/z: 446 [M+NH₄]⁺.

To a mixture of 6-(4-(benzyloxy)-5-bromo-2-chlorobenzyl)-2,3-dihydrobenzofuran (438 mg, 1.02 mmol, 1.00 equiv) in tetrahydrofuran (5 mL) was added n-BuLi (0.41 mL, 2.5M/L) dropwise at −78° C. 30 min later, to the mixture was added a solution of (3R,4R,6R)-3,4-bis(benzyloxy)-6-[(benzyloxy)methyl]-5,5-difluorooxan-2-one (400 mg, 0.85 mmol, 0.84 equiv) in tetrahydrofuran (0.5 mL) dropwise at −78° C. The reaction mixture was stirred at −78° C. for 2 h, NH₄Cl/H₂O was added and the mixture was extracted with EtOAc thrice. The combined extracts were washed with brine and dried over Na₂SO₄. The mixture was concentrated and the resulting residue purified by chromatography on silica gel (3:1 PE/EA) to yield (3R,4R,6R)-3,4-bis(benzyloxy)-2-(2-(benzyloxy)-4-chloro-5-((2,3-dihydrobenzofuran-6-yl)methyl)phenyl)-6-((benzyloxy)methyl)-5,5-difluorotetrahydro-2H-pyran-2-ol as a colorless oil. MS (ES) m/z: 836[M+NH₄]⁺.

To a mixture of (3R,4R,6R)-3,4-bis(benzyloxy)-2-(2-(benzyloxy)-4-chloro-5-((2,3-dihydrobenzofuran-6-yl)methyl)phenyl)-6-((benzyloxy)methyl)-5,5-difluorotetrahydro-2H-pyran-2-ol (805 mg, 0.98 mmol, 1.00 equiv) in dichloromethane (10 mL) with Et₃SiH (248 mg, 2.13 mmol, 2.17 equiv) was added BF₃.Et₂O (227 mg) dropwise at 0° C. The reaction mixture was stirred for 1 h at 0° C., NaHCO₃/H₂O was added and the mixture was extracted with EtOAc thrice. The combined extracts were washed with brine and dried over Na₂SO₄. The mixture was concentrated and the resulting residue purified by chromatography on silica gel (3:1 PE/EA) to yield 6-(4-(benzyloxy)-5-((2S,3S,4R,6R)-3,4-bis(benzyloxy)-6-((benzyloxy)methyl)-5,5-difluorotetrahydro-2H-pyran-2-yl)-2-chlorobenzyl)-2,3-dihydrobenzofuran as a colorless oil. MS (ES) m/z: 820[M+NH₄]⁺.

To a mixture of 6-(4-(benzyloxy)-5-((2S,3S,4R,6R)-3,4-bis(benzyloxy)-6-((benzyloxy)methyl)-5,5-difluorotetrahydro-2H-pyran-2-yl)-2-chlorobenzyl)-2,3-dihydrobenzofuran (600 mg, 0.75 mmol, 1.00 equiv) in EA/MeOH (6/2 mL) was added Pd(OH)₂/C (500 mg), Hydrogen was introduced into this solution, The reaction mixture was stirred for 20 min at room temperature. The solids were filtered out. The resulting mixture was concentrated and the residue purified by Prep-HPLC to yield (2S,3R,4R,6R)-2-(4-chloro-5-((2,3-dihydrobenzofuran-6-yl)methyl)-2-hydroxyphenyl)-5,5-difluoro-6-(hydroxymethyl)tetrahydro-2H-pyran-3,4-diol, the title compound, as a white solid.

¹H NMR (300 MHz, Methanol-d₄) δ: 7.28 (s, 1H), 7.07 (d, J=7.5 Hz, 1H), 6.86 (s, 1H), 6.66 (d, J=9.0 Hz, 1H), 6.52 (s, 1H), 4.70 (d, J=9.3 Hz, 1H), 4.50 (t, J=9.0 Hz, 2H), 3.69-3.92 (m, 7H), 3.11 (t, J=7.8 Hz, 2H). MS (ES) m/z: 441[M−H]⁻.

Additional representative compounds of the present invention, detailed in Table 4, below, were similarly prepared according to the procedures described in Examples 3-5 above, selecting and substituting suitably substituted reagents and starting materials, as would be readily recognized by those skilled in the art.

TABLE 4
Representative Compounds of Formjula (I)

| ID No. | Compound name/Structural formula/Analysis data |
|---|---|
| 45 | (2S,3R,4R,6R)-2-[4-chloro-5-(2,3-dihydro-1,4-benzodioxin-6- |
| | ylmethyl)-2-hydroxy-phenyl]-5,5-difluoro-6- |
| | (hydroxymethyl)tetrahydropyran-3,4-diol |
| | ¹H NMR (METHANOL-d₄) δ: 7.21 (s, 1H), 6.85 (s, 1H), 6.67-6.71 (m, |
| | 1H), 6.58-6.64 (m, 2H), 4.69 (d, J = 9.6 Hz, 1H), 4.17 (s, 4H), 3.68- |
| | 3.92 ( m, 7H). MS (ESI⁺, m/z): [M + Na]⁺ m/z 481.05 |
| 48 | (2S,3R,4R,6R)-2-[4-chloro-5-(2,3-dihydro-1,4-benzodioxin-6- |
| | ylmethyl)-2-methoxy-phenyl]-5,5-difluoro-6- |
| | (hydroxymethyl)tetrahydropyran-3,4-diol |
| | ¹H NMR (METHANOL-d₄) δ: 7.27 (s, 1H), 7.02 (s, 1H), 6.68-6.73 (m, |
| | 1H), 6.59-6.65 (m, 2H), 4.73 (br d, J = 9.6 Hz, 1H), 4.18 (s, 4H), 3.65- |
| | 3.94 (m, 10H). MS (ESI⁺, m/z): [M + Na]⁺ m/z 495.15 |
| 54 | (2S,3R,4R,6R)-2-[5-(2,3-dihydro-1,4-benzodioxin-6-ylmethyl)-2- |
| | hydroxy-4-methoxy-phenyl]-5,5-difluoro-6- |
| | (hydroxymethyl)tetrahydropyran-3,4-diol |
| | ¹H NMR (MeOH) δ: 7.04 (s, 1H), 6.59-6.66 (m, 3H), 6.43 (s, 1H), 4.64 |
| | (d, J = 9.6 Hz, 1H), 4.16 (s, 4H), 3.69-3.90 (m, 10H). MS (ESI⁺, m/z): |
| | [M + Na]⁺ m/z 447.15 |
| 55 | (2S,3R,4R,6R)-2-[5-(2,3-dihydro-1,4-benzodioxin-6-ylmethyl)-2,4- |
| | dimethoxy-phenyl]-5,5-difluoro-6-(hydroxymethyl)tetrahydropyran-3,4- |
| | diol |
| | ¹H NMR (METHANOL-d₄) δ: 7.27-7.33 (m, 1H), 7.10 (s, 1H), 6.59- |
| | 6.68 (m, 3H), 4.69 (br d, J = 9.1 Hz, 1H), 4.16 (s, 4H), 3.69-3.89 (m, |
| | 13H). MS (ESI⁺, m/z): [M + Na]⁺ m/z 491.15 |
| 62 | (2S,3R,4R,6R)-2-[5-(2,3-dihydrobenzofuran-5-ylmethyl)-2-hydroxy-4- |
| | methoxy-phenyl]-5,5-difluoro-6-(hydroxymethyl)tetrahydropyran-3,4- |
| | diol |
| | ¹H NMR (MeOH) δ: 7.04 (s, 1H), 6.99 (s, 1H), 6.88 (d, J = 7.6 Hz, 1H), |
| | 6.55 (d, J = 8.1 Hz, 1H), 6.43 (s, 1H), 4.64 (d, J = 9.1 Hz, 1H), 4.46 (t, |
| | J = 8.8 Hz, 2H), 3.70-3.83 (m, 7H), 3.12 (d, J = 7.6 Hz, 2H). MS(ESI⁺, |
| | m/z): [M + Na]⁺ m/z 461.25 |

### Example 6: Compound #88

### (2S,3R,4R,6R)-2-(4-ethyl-2-hydroxy-5-(4-methoxybenzyl)phenyl)-5,5-difluoro-6-(hydroxymethyl)-tetrahydro-2H-pyran-3,4-diol

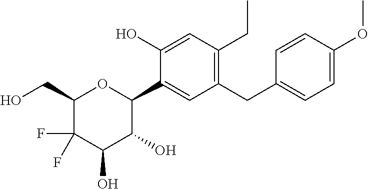 [SMILES: CCc1cc(O)c([C@@H]2O[C@H](CO)C(F)(F)[C@H](O)[C@H]2O)cc1Cc1ccc(OC)cc1]

To a mixture of 2-(4-(benzyloxy)-5-bromo-2-ethylphenyl)-1,3-dioxolane (2 g, 5.52 mmol, 1.00 equiv) in tetrahydrofuran/toluene (7/14 mL) was added n-BuLi (2.2 mL, 5.50 mmol, 1.00 equiv, 2.5 M in hexane) dropwise at −78° C. The reaction mixture was stirred for 30 min at −78° C. To the mixture was then added a solution of (3R,4S,6S)-3,4-bis(benzyloxy)-6-[(benzyloxy)methyl]-5-methylideneoxan-2-one (2.2 g, 4.95 mmol, 0.90 equiv) in tetrahydrofuran (3 mL) dropwise at −78° C. The reaction mixture was stirred for 2 h at −78° C. NH₄Cl/H₂O was added and the mixture was extracted with EtOAc thrice. The combined extracts were washed with brine and dried over Na₂SO₄. The mixture was concentrated and the resulting residue purified by chromatography on silica gel (3:1 PE/EA) to yield (3R,4S,6S)-3,4-bis(benzyloxy)-2-(2-(benzyloxy)-5-(1,3-dioxolan-2-yl)-4-ethylphenyl)-6-((benzyloxy)methyl)-5-methylenetetrahydro-2H-pyran-2-ol as a colorless oil.

To a mixture of (3R,4S,6S)-3,4-bis(benzyloxy)-2-(2-(benzyloxy)-5-(1,3-dioxolan-2-yl)-4-ethylphenyl)-6-((benzyloxy)methyl)-5-methylenetetrahydro-2H-pyran-2-ol (3.6 g, 4.94 mmol, 1.00 equiv) in THE (50 mL) was added 2N HCl (10 mL) dropwise at 0° C. The reaction mixture was stirred for 2 h at room temperature, Water was added and the mixture was extracted with EtOAc thrice. The combined extracts were washed with brine and dried over Na₂SO₄. The mixture was concentrated and the resulting residue purified by chromatography on silica gel (3:1 PE/EA) to yield 4-(benzyloxy)-5-((3R,4S,6S)-3,4-bis(benzyloxy)-6-((benzyloxy)methyl)-2-hydroxy-5-methylenetetrahydro-2H-pyran-2-yl)-2-ethylbenzaldehyde as a colorless oil.

To a mixture of 1-bromo-4-methoxybenzene (2 g, 10.75 mmol, 3.41 equiv) in THE (40 mL) was added n-BuLi (4.67 mL, 11.68 mmol, 3.71 equiv, 2.5 M in hexane) dropwise at −78° C. The reaction mixture was stirred for 30 min at −78° C. To the mixture was then added a solution of 4-(benzyloxy)-5-((3R,4S,6S)-3,4-bis(benzyloxy)-6-((benzyloxy)methyl)-2-hydroxy-5-methylenetetrahydro-2H-pyran-2-yl)-2-ethylbenzaldehyde (2.16 g, 3.15 mmol, 1.00 equiv) in THE (2 mL) dropwise at −78° C. The reaction mixture was stirred for 2 h at −78° C., NH₄Cl/H₂O was added and the mixture was extracted with EtOAc thrice. The combined extracts were washed with brine and dried over Na₂SO₄. The mixture was concentrated and the resulting residue purified by chromatography on silica gel (2:1 PE/EA) to yield (3R,4S,6S)-3,4-bis(benzyloxy)-2-(2-(benzyloxy)-4-ethyl-5-(hydroxy(4-methoxyphenyl)methyl)phenyl)-6-((benzyloxy)methyl)-5-methylenetetrahydro-2H-pyran-2-ol as a colorless oil.

To a mixture of (3R,4S,6S)-3,4-bis(benzyloxy)-2-(2-(benzyloxy)-4-ethyl-5-(hydroxy(4-methoxyphenyl)methyl)phenyl)-6-((benzyloxy)methyl)-5-methylenetetrahydro-2H-pyran-2-ol (2 g, 2.52 mmol, 1.00 equiv) in dichloromethane (25 mL) with Et₃SiH (1.16 g, 9.98 mmol, 3.96 equiv) was added BF₃.Et₂O (1.07 g, 7.54 mmol, 3.00 eq) dropwise at 0° C. The reaction mixture was stirred for 1 h at 0° C., NaHCO₃/H₂O was added and the mixture was extracted with DCM thrice. The combined extracts were washed with brine and dried over Na₂SO₄. The mixture was concentrated and the resulting residue purified by chromatography on silica gel (3:1 PE/EA) to yield (2S,3R,4S,6S)-3,4-bis(benzyloxy)-2-(2-(benzyloxy)-4-ethyl-5-(4-methoxybenzyl)phenyl)-6-((benzyloxy)methyl)-5-methylenetetrahydro-2H-pyran as a colorless oil.

To a mixture of (2S,3R,4S,6S)-3,4-bis(benzyloxy)-2-(2-(benzyloxy)-4-ethyl-5-(4-methoxybenzyl)phenyl)-6-((benzyloxy)methyl)-5-methylenetetrahydro-2H-pyran (1.2 g, 1.58 mmol, 1.00 equiv) and NMO (550 mg, 4.69 mmol, 2.98 equiv) in Acetone/H₂O (20/2 mL) was added OsO₄ (2 mL, 2.5 wt % in t-BuOH) dropwise. The reaction mixture was stirred for 16 h at room temperature, Na₂S₂O₃ was added and the mixture was extracted with EtOAc thrice. The combined extracts were washed with brine and dried over Na₂SO₄. The mixture was concentrated and the resulting residue purified by chromatography on silica gel (2:1 PE/EA) to yield (2R,4R,5S,6S)-4,5-bis(benzyloxy)-6-(2-(benzyloxy)-4-ethyl-5-(4-methoxybenzyl)phenyl)-2-((benzyloxy)methyl)-3-(hydroxymethyl)tetrahydro-2H-pyran-3-ol as a colorless oil. MS (ES) m/z: 812 [M+NH₄]⁺.

To a mixture of (2R,4R,5S,6S)-4,5-bis(benzyloxy)-6-(2-(benzyloxy)-4-ethyl-5-(4-methoxybenzyl)phenyl)-2-((benzyloxy)methyl)-3-(hydroxymethyl)tetrahydro-2H-pyran-3-ol (600 mg, 0.75 mmol, 1.00 equiv) in dichloromethane (5 mL) was added PhI(OAc)₂ (443 mg, 1.38 mmol, 1.82 equiv). The reaction mixture was stirred for 16 h at room temperature, H₂O was added and the mixture was extracted with EtOAc thrice. The combined extracts were washed with brine and dried over Na₂SO₄. The mixture was concentrated and the resulting residue purified by chromatography on silica gel (3:1 PE/EA) to yield (2R,4R,5S,6S)-4,5-bis(benzyloxy)-6-(2-(benzyloxy)-4-ethyl-5-(4-methoxybenzyl)phenyl)-2-((benzyloxy)methyl)dihydro-2H-pyran-3(4H)-one as a colorless oil. MS (ES) m/z: 780 [M+NH₄]⁺.

To a mixture of (2R,4R,5S,6S)-4,5-bis(benzyloxy)-6-(2-(benzyloxy)-4-ethyl-5-(4-methoxybenzyl)phenyl)-2-((benzyloxy)methyl)dihydro-2H-pyran-3(4H)-one (400 mg, 0.52 mmol, 1.00 equiv) in dichloromethane (10 mL) was added DAST (340 mg, 9.17 mmol, 17.48 equiv) at 0° C., The reaction mixture was stirred for 16 h at room temperature. MeOH was added and the mixture was extracted with DCM thrice. The combined extracts were washed with brine and dried over Na₂SO₄. The mixture was concentrated and the resulting residue purified by chromatography on silica gel (2:1 PE/EA) to yield (2R,4R,5S,6S)-4,5-bis(benzyloxy)-6-(2-(benzyloxy)-4-ethyl-5-(4-methoxybenzyl)phenyl)-2-((benzyloxy)methyl)-3,3-difluorotetrahydro-2H-pyran as a colorless oil. MS (ES) m/z: 802 [M+NH₄]⁺.

To a mixture of (2R,4R,5S,6S)-4,5-bis(benzyloxy)-6-(2-(benzyloxy)-4-ethyl-5-(4-methoxybenzyl)phenyl)-2-((benzyloxy)methyl)-3,3-difluorotetrahydro-2H-pyran (350 mg, 0.45 mmol, 1.00 equiv) in EA/MeOH (3/1 mL) was added Pd(OH)₂/C (100 mg), H₂ was introduced into. The reaction mixture was stirred at room temperature for 30 min. The mixture was filtered, the filtrate concentrated and the resulting residue purified by chromatography on C18 reverse column to yield (2S,3R,4R,6R)-2-(4-ethyl-2-hydroxy-5-(4-methoxybenzyl)phenyl)-5,5-difluoro-6-(hydroxymethyl)tetrahydro-2H-pyran-3,4-diol, the title compound, as a white solid.

¹H NMR (300 MHz, CD₃OD) δ: 7.11 (s, 1H), 7.02 (d, J=8.6 Hz, 2H), 6.79 (d, J=8.6 Hz, 2H), 6.69 (s, 1H), 4.70 (d, J=9.2 Hz, 1H), 3.71-3.95 (m, 10H), 2.50 (q, J=7.5 Hz, 2H), 1.05 (t, J=7.5 Hz, 3H). MS (ES) m/z: 423.1 [M−H]⁻.

### Example 7: Compound #99

### (2S,3R,4R,6R)-5,5-difluoro-2-(2-hydroxy-4-methyl-5-(4-(trifluoromethoxy)benzyl)phenyl)-6-(hydroxymethyl)-tetrahydro-2H-pyran-3,4-diol

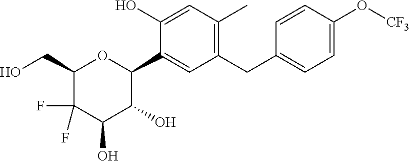 [SMILES: Cc1cc(O)c([C@@H]2O[C@H](CO)C(F)(F)[C@H](O)[C@H]2O)cc1Cc1ccc(OC(F)(F)F)cc1]

To a mixture of 2-(4-(benzyloxy)-5-bromo-2-methylphenyl)-1,3-dioxolane (2 g, 5.73 mmol, 1.00 equiv) in tetrahydrofuran/toluene (8/16 mL) was added n-BuLi (2.30 mL, 5.75 mmol, 1.00 equiv, 2.5 M in hexane) dropwise at −78° C., and the solution was stirred for 30 min at −78° C. To the mixture was then added (3R,4S,6S)-3,4-bis(benzyloxy)-6-[(benzyloxy)methyl]-5-methylideneoxan-2-one (2.55 g, 5.73 mmol, 1.00 equiv). The reaction mixture was stirred for 2 h at −78° C., NH₄Cl/H₂O was added and the mixture was extracted with EtOAc thrice. The combined extracts were washed with brine and dried over Na₂SO₄. The mixture was concentrated and the resulting residue purified by chromatography on silica gel (4:1 PE/EA) to yield (3R,4S,6S)-3,4-bis(benzyloxy)-2-(2-(benzyloxy)-5-(1,3-dioxolan-2-yl)-4-methylphenyl)-6-((benzyloxy)methyl)-5-methylenetetrahydro-2H-pyran-2-ol as a yellow oil.

To a mixture of (3R,4S,6S)-3,4-bis(benzyloxy)-2-(2-(benzyloxy)-5-(1,3-dioxolan-2-yl)-4-methylphenyl)-6-((benzyloxy)methyl)-5-methylenetetrahydro-2H-pyran-2-ol (3 g, 4.20 mmol, 1.00 equiv) in tetrahydrofuran (30 ml) was added 2N hydrogen chloride (6 mL). The reaction mixture was stirred for 1 h at room temperature, H₂O was added and the mixture was extracted with EtOAc thrice. The combined extracts were washed with brine and dried over Na₂SO₄. The mixture was concentrated and the resulting residue purified by chromatography on silica gel (3:1 PE/EA) to yield 4-(benzyloxy)-5-((3R,4S,6S)-3,4-bis(benzyloxy)-6-((benzyloxy)methyl)-2-hydroxy-5-methylenetetrahydro-2H-pyran-2-yl)-2-methylbenzaldehyde as a yellow oil.

To a mixture of 1-bromo-4-(trifluoromethoxy)benzene (2.37 g, 9.85 mmol, 3.00 equiv) in tetrahydrofuran (25 ml) was added n-BuLi (3.94 mL, 9.85 mmol, 3.00 equiv) dropwise at −78° C. The solution was stirred for 30 min at −78° C. To the mixture was then added 4-(benzyloxy)-5-((3R,4S,6S)-3,4-bis(benzyloxy)-6-((benzyloxy)methyl)-2-hydroxy-5-methylenetetrahydro-2H-pyran-2-yl)-2-methylbenzaldehyde (2.2 g, 3.28 mmol, 1.00 equiv). The reaction mixture was stirred at −78° C. for 2 h, NH₄Cl/H₂O was added and the mixture was extracted with EtOAc thrice. The combined extracts were washed with brine and dried over Na₂SO₄. The mixture was concentrated and the resulting residue purified by chromatography on silica gel (3:1 PE/EA) to yield (3R,4S,6S)-3,4-bis(benzyloxy)-2-(2-(benzyloxy)-5-(hydroxy(4-(trifluoromethoxy)phenyl)methyl)-4-methylphenyl)-6-((benzyloxy)methyl)-5-methylenetetrahydro-2H-pyran-2-ol as a yellow oil.

To a mixture of (3R,4S,6S)-3,4-bis(benzyloxy)-2-(2-(benzyloxy)-5-(hydroxy(4-(trifluoromethoxy)phenyl)methyl)-4-methylphenyl)-6-((benzyloxy)methyl)-5-methylenetetrahydro-2H-pyran-2-ol (2 g, 2.40 mmol, 1.00 equiv) in dichloromethane (20 mL) with Et₃SiH (1.12 g, 9.63 mmol, 4.01 equiv) was added BF₃.Et₂O (1.37 g, 9.65 mmol, 4.02 equiv) at 0° C. The reaction mixture was stirred for 1 h at 0° C., NaHCO₃/H₂O was added and the mixture was extracted with EtOAc thrice. The combined extracts were washed with brine and dried over Na₂SO₄. The mixture was concentrated and the resulting residue purified by chromatography on silica gel (4:1 PE/EA) to yield (2S,3R,4S,6S)-3,4-bis(benzyloxy)-2-(2-(benzyloxy)-4-methyl-5-(4-(trifluoromethoxy)benzyl)phenyl)-6-((benzyloxy)methyl)-5-methylenetetrahydro-2H-pyran as a yellow oil. MS (ES) m/z: 823.2 [M+Na]⁺.

To a mixture of (2S,3R,4S,6S)-3,4-bis(benzyloxy)-2-(2-(benzyloxy)-4-methyl-5-(4-(trifluoromethoxy)benzyl)phenyl)-6-((benzyloxy)methyl)-5-methylenetetrahydro-2H-pyran (1 g, 1.25 mmol, 1.00 equiv) in acetone/H₂O (20/3 ml) with NMO (439 mg, 3.75 mmol, 3.00 equiv) was added OsO₄ (6.35 mg, 0.02 equiv) at room temperature, The reaction mixture was stirred for 4 h at 45° C. Ice water was added and the mixture was extracted with EtOAc thrice. The combined extracts were washed with H₂O, brine and dried over Na₂SO₄. The mixture was concentrated and the resulting residue purified by chromatography on silica gel (4:1 PE/EA) to yield (2R,4R,5S,6S)-4,5-bis(benzyloxy)-6-(2-(benzyloxy)-4-methyl-5-(4-(trifluoromethoxy)benzyl)phenyl)-2-((benzyloxy)methyl)-3-(hydroxymethyl)tetrahydro-2H-pyran-3-ol as an off-white solid. MS (ES) m/z: 835.1 [M+H]⁺.

To a mixture of (2R,4R,5S,6S)-4,5-bis(benzyloxy)-6-(2-(benzyloxy)-4-methyl-5-(4-(trifluoromethoxy)benzyl)phenyl)-2-((benzyloxy)methyl)-3-(hydroxymethyl)tetrahydro-2H-pyran-3-ol (1.2 g, 1.44 mmol, 1.00 equiv) in dichloromethane (20 ml) was added PhI(OAc)₂ (930 mg, 2.89 mmol, 2.00 equiv) at room temperature, The reaction mixture was stirred for 2h at room temperature. Ice water was added and the mixture was extracted with EtOAc thrice. The combined extracts were washed with H₂O, brine and dried over Na₂SO₄, The mixture was concentrated and the resulting residue purified by chromatography on silica gel (3:1 PE/EA) to yield (2R,4R,5S,6S)-4,5-bis(benzyloxy)-6-(2-(benzyloxy)-4-methyl-5-(4-(trifluoromethoxy)benzyl)phenyl)-2-((benzyloxy)methyl)dihydro-2H-pyran-3(4H)-one yellow oil. MS (ES) m/z: 820.1 [M+NH₄]⁺

To a mixture of (2R,4R,5S,6S)-4,5-bis(benzyloxy)-6-(2-(benzyloxy)-4-methyl-5-(4-(trifluoromethoxy)benzyl)phenyl)-2-((benzyloxy)methyl)dihydro-2H-pyran-3(4H)-one (1 g, 1.25 mmol, 1.00 equiv) in dichloromethane (15 ml) was added DAST (1 g, 26.96 mmol, 5.00 equiv), The reaction mixture was stirred for overnight at room temperature. Ice water was added and the mixture was extracted with DCM thrice. The combined extracts were washed with H₂O, brine and dried over Na₂SO₄. The mixture was concentrated and the resulting residue purified by chromatography on silica gel (4:1 PE/EA) to yield (2R,4R,5S,6S)-4,5-bis(benzyloxy)-6-(2-(benzyloxy)-4-methyl-5-(4-(trifluoromethoxy)benzyl)phenyl)-2-((benzyloxy)methyl)-3,3-difluorotetrahydro-2H-pyran as a yellow solid. MS (ES) m/z: 842.1 [M+NH₄]⁺

To a mixture of (2R,4R,5S,6S)-4,5-bis(benzyloxy)-6-(2-(benzyloxy)-4-methyl-5-(4-(trifluoromethoxy)benzyl)phenyl)-2-((benzyloxy)methyl)-3,3-difluorotetrahydro-2H-pyran (300 mg, 0.36 mmol, 1.00 equiv) in EA/MeOH (9/3 mL) was added Pd(OH)₂/C (100 mg), H₂ was introduced into. The reaction mixture was stirred at room temperature for 1 h. The mixture was filtered, the filtrated concentrated and the resulting residue purified by chromatography on C18 reverse column to yield (2S,3R,4R,6R)-5,5-difluoro-2-(2-hydroxy-4-methyl-5-(4-(trifluoromethoxy)benzyl)phenyl)-6-(hydroxymethyl)tetrahydro-2H-pyran-3,4-diol, the title compound, as a white solid.

¹H NMR (300 MHz, Methanol-d₄) δ: 7.16-7.30 (m, 5H), 7.02 (s, 1H), 6.62 (s, 1H), 5.75 (d, J=6.1 Hz, 1H), 5.18 (s, 1H), 4.85 (t, J=5.8 Hz, 1H), 4.60 (d, J=9.5 Hz, 1H), 3.89 (s, 2H), 3.53-3.79 (m, 4H), 3.51-3.40 (m, 1H), 2.05 (s, 3H). MS (ES) m/z: 463.0 [M−H]⁻.

### Example 8: Compound #81

### (2S,3R,4R,6R)-2-(4-chloro-2-hydroxy-5-(4-methoxybenzyl)phenyl)-5,5-difluoro-6-(hydroxymethyl)-tetrahydro-2H-pyran-3,4-diol

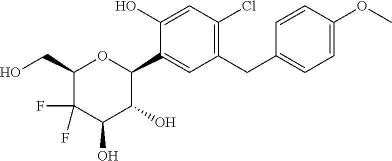 [SMILES: COc1ccc(Cc2cc([C@@H]3O[C@H](CO)C(F)(F)[C@H](O)[C@H]3O)c(O)cc2Cl)cc1]

To a mixture of 1-(benzyloxy)-2-bromo-5-chloro-4-(4-methoxybenzyl)benzene (6.36 g, 15.23 mmol, 1.10 equiv) in tetrahydrofuran/toluene (1:2) (90 mL) was added n-BuLi (6.1 mL, 1.10 equiv, 2.5N) dropwise at −78° C. The solution was stirred for 30 min at −78° C. To the mixture was then added a solution of (3R,4R,6R)-3,4-bis(benzyloxy)-6-[(benzyloxy)methyl]-5,5-difluorooxan-2-one (6.5 g, 13.87 mmol, 1.0 equiv) in tetrahydrofuran (50 mL) dropwise at −78° C. The reaction mixture was stirred at −78° C. for 1 h. NH₄Cl/H₂O was added and the mixture was extracted with EtOAc thrice. The combined extracts were washed with brine and dried over Na₂SO₄. The mixture was concentrated and the resulting residue purified by chromatography on silica gel (5:1 PE/EA) to yield (3R,4R,6R)-3,4-bis(benzyloxy)-2-(2-(benzyloxy)-4-chloro-5-(4-methoxybenzyl)phenyl)-6-((benzyloxy)methyl)-5,5-difluorotetrahydro-2H-pyran-2-ol as a yellow oil.

To a mixture of (3R,4R,6R)-3,4-bis(benzyloxy)-2-(2-(benzyloxy)-4-chloro-5-(4-methoxybenzyl)phenyl)-6-((benzyloxy)methyl)-5,5-difluorotetrahydro-2H-pyran-2-ol (9.0 g, 0.11 mmol, 1.00 equiv) in DCM/MeCN (1:1, 100 ml) with Et₃SiH (2.60 g, 0.22 mol, 2.00 equiv) was added BF₃.Et₂O (3.17 g, 0.22 mol, 2.00 equiv) dropwise at 0° C. The reaction mixture was stirred for 1 h at 0° C. sodium bicarbonate/H₂O was added and the mixture was extracted with DCM thrice. The combined extracts were washed with brine and dried over Na₂SO₄. The mixture was concentrated and the resulting residue purified by chromatography on silica gel (5:1 PE/EA) to yield (2R,4R,5S,6S)-4,5-bis(benzyloxy)-6-(2-(benzyloxy)-4-chloro-5-(4-methoxybenzyl)phenyl)-2-((benzyloxy)methyl)-3,3-difluorotetrahydro-2H-pyran as a yellow oil.

To a mixture of (2R,4R,5S,6S)-4,5-bis(benzyloxy)-6-(2-(benzyloxy)-4-chloro-5-(4-methoxybenzyl)phenyl)-2-((benzyloxy)methyl)-3,3-difluorotetrahydro-2H-pyran (4.0 g, 5.05 mmol, 1.00 equiv) in EA/MeOH (3:1) (100 mL) was added Pd(OH)₂/C (2.0 g). Hydrogen was introduced. The reaction mixture was stirred for 1 h at room temperature. The solids were filtered out. The mixture was concentrated and the resulting residue purified by chromatograph on a C18 reversed phase column to yield (2S,3R,4R,6R)-2-(4-chloro-2-hydroxy-5-(4-methoxybenzyl)phenyl)-5,5-difluoro-6-(hydroxymethyl)tetrahydro-2H-pyran-3,4-diol, the title compound, as a white solid.

¹H-NMR (300 MHz, CD₃OD) δ: 7.22 (s, 1H), 7.08 (d, J=8.7 Hz, 2H), 6.78-6.83 (m, 3H), 4.70 (d, J=9.5 Hz, 1H), 3.64-4.03 (m, 10H). MS(ES) 448.1 [M+NH₄]⁺

### Example 9: Compound #47

### (2S,3R,4R,6R)-2-(5-(4-ethoxybenzyl)-2-hydroxy-4-methylphenyl)-5,5-difluoro-6-(hydroxymethyl)tetrahydro-2H-pyran-3,4-diol

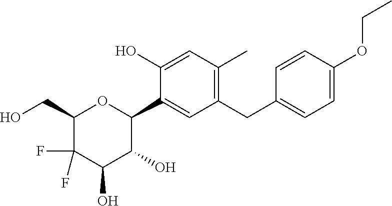 [SMILES: CCOc1ccc(Cc2cc([C@@H]3O[C@H](CO)C(F)(F)[C@H](O)[C@H]3O)c(O)cc2C)cc1]

To a mixture of 1-bromo-4-ethoxybenzene (2 g, 9.95 mmol, 1.00 equiv) in THE (20 mL) was added n-BuLi (2.5M in hexane, 3.98 mL, 1.00 equiv) dropwise at −78° C., the mixture was stirred at −78° C. for 30 min. 4-(Benzyloxy)-5-bromo-2-methylbenzaldehyde (3.025 g, 9.91 mmol, 1.00 equiv) in THE (10 mL) was then added to the solution. The reaction mixture was stirred at −78° C. for 2h. NH₄Cl/H₂O was added and the mixture was extracted with EtOAc thrice. The combined extracts were washed with brine and dried over Na₂SO₄. The mixture was concentrated and the resulting residue purified by chromatography on silica gel (3:1 PE/EA) to yield (4-(benzyloxy)-5-bromo-2-methylphenyl)(4-ethoxyphenyl)methanol as a yellow oil.

To a mixture of (4-(benzyloxy)-5-bromo-2-methylphenyl)(4-ethoxyphenyl)methanol (4 g, 9.36 mmol, 1.00 equiv) in DCM (40 mL) with Et₃SiH (1.09 g, 9.37 mmol, 1.00 equiv) was added BF₃.Et₂O (1.33 g, 9.37 mmol, 1.00 equiv) at 0° C. The reaction mixture was stirred at 0° C. for 1h. Sodium bicarbonate/H₂O was added and the mixture was extracted with dichloromethane thrice. The combined extracts were washed with brine and dried over Na₂SO₄. The mixture was concentrated and the resulting residue purified by chromatography on silica gel (4:1 PE/EA) to yield 1-(benzyloxy)-2-bromo-4-(4-ethoxybenzyl)-5-methylbenzene as a yellow oil.

To a mixture of 1-(benzyloxy)-2-bromo-4-(4-ethoxybenzyl)-5-methylbenzene (410 mg, 1.00 mmol, 1.00 equiv) in THE (5 mL) was added n-BuLi (2.5M in hexane, 0.41 mL, 1.00 equiv) dropwise at −78° C., the mixture was stirred at −78° C. for 20 min. (3R,4R,6R)-3,4-Bis(benzyloxy)-6-[(benzyloxy)methyl]-5,5-difluorooxan-2-one (500 mg, 1.07 mmol, 1.00 equiv) in THE (2 mL) was then added to the solution. The reaction mixture was stirred at −78° C. for 2h. NH₄Cl/H₂O was added and the mixture was extracted with EtOAc thrice. The combined extracts were washed with brine and dried over Na₂SO₄. The mixture was concentrated and the resulting residue purified by chromatography on silica gel (3:1 PE/EA) to yield (3R,4R,6R)-3,4-bis(benzyloxy)-2-(2-(benzyloxy)-5-(4-ethoxybenzyl)-4-methylphenyl)-6-((benzyloxy)methyl)-5,5-difluorotetrahydro-2H-pyran-2-ol as a yellow oil.

To a mixture of (3R,4R,6R)-3,4-bis(benzyloxy)-2-(2-(benzyloxy)-5-(4-ethoxybenzyl)-4-methylphenyl)-6-((benzyloxy)methyl)-5,5-difluorotetrahydro-2H-pyran-2-ol (1 g, 1.25 mmol, 1.00 equiv) in DCM (15 mL) with Et₃SiH (430 mg, 3.70 mmol, 3.00 equiv) was added BF₃.Et₂O (0.352 g, 2.48 mmol, 2.00 equiv) at 0° C. The reaction mixture was stirred at 0° C. for 1h. Sodium bicarbonate/H₂O was added and the mixture was extracted with dichloromethane thrice. The combined extracts were washed with brine and dried over Na₂SO₄. The mixture was concentrated and the resulting residue purified by chromatography on silica gel (3:1 PE/EA) to yield (2R,4R,5S,6S)-4,5-bis(benzyloxy)-6-(2-(benzyloxy)-5-(4-ethoxybenzyl)-4-methylphenyl)-2-((benzyloxy)methyl)-3,3-difluorotetrahydro-2H-pyran as a yellow oil.

To a solution of (2R,4R,5S,6S)-4,5-bis(benzyloxy)-6-(2-(benzyloxy)-5-(4-ethoxybenzyl)-4-methylphenyl)-2-((benzyloxy)methyl)-3,3-difluorotetrahydro-2H-pyran (400 mg, 0.51 mmol, 1.00 equiv) in EA/MeOH (20/10 mL) was added Pd(OH)₂/C (20 mg). H₂ was introduced in. The reaction mixture was stirred for 30 min at room temperature. The solids were filtered out. The filtrate was concentrated and the resulting residue purified by chromatography on silica gel (20:1 DCM/MeOH) to yield (2S,3R,4R,6R)-2-(5-(4-ethoxybenzyl)-2-hydroxy-4-methylphenyl)-5,5-difluoro-6-(hydroxymethyl)tetrahydro-2H-pyran-3,4-diol, the title compound, as a white solid.

¹H NMR (300 MHz, CD₃OD) δ: 7.13 (s, 1H), 7.02 (d, J=8.4 Hz, 2H), 6.80 (d, J=8.7 Hz, 2H), 6.66 (s, 1H), 4.72 (d, J=9.1 Hz, 1H), 3.99 (t, J=6.9 Hz, 1H), 3.75-3.93 (m, 7H), 2.13 (s, 3H), 1.38 (t, J=7.0 Hz, 3H). MS (ES) m/z: 407.1 [M−H]-.

### Example 10: Compound #91

### (2S,3R,4R,6R)-5,5-difluoro-2-(2-hydroxy-4-methoxy-5-(4-methoxybenzyl)phenyl)-6-(hydroxymethyl)-tetrahydro-2H-pyran-3,4-diol

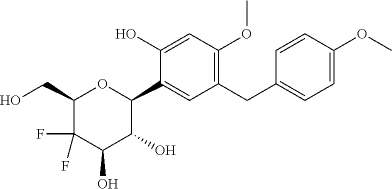 [SMILES: COc1ccc(Cc2cc([C@@H]3O[C@H](CO)C(F)(F)[C@H](O)[C@H]3O)c(O)cc2OC)cc1]

To a mixture of 1-(benzyloxy)-2-bromo-5-methoxy-4-(4-methoxybenzyl)benzene (2.64 g, 6.39 mmol, 1.00 equiv) in tetrahydrofuran (45 mL) was added n-BuLi (2.57 mL, 1.00 equiv, 2.5N) dropwise at −78° C. The solution was stirred for 30 min at −78° C. To the mixture was then added a solution of (3R,4R,6R)-3,4-bis(benzyloxy)-6-[(benzyloxy)methyl]-5,5-difluorooxan-2-one (3.0 g, 6.40 mmol, 1.00 equiv) in tetrahydrofuran (10 mL) dropwise at −78° C. The reaction mixture was stirred for 1 h at −78° C. NH₄Cl/H₂O was added and the mixture was extracted with EtOAc thrice. The combined extracts were washed with brine and dried over Na₂SO₄. The mixture was concentrated and the resulting residue purified by chromatography on silica gel (3:1 PE/EA) to yield (3R,4R,6R)-3,4-bis(benzyloxy)-2-(2-(benzyloxy)-4-methoxy-5-(4-methoxybenzyl)phenyl)-6-((benzyloxy)methyl)-5,5-difluorotetrahydro-2H-pyran-2-ol as a yellow oil.

To a mixture of (3R,4R,6R)-3,4-bis(benzyloxy)-2-(2-(benzyloxy)-4-methoxy-5-(4-methoxybenzyl)phenyl)-6-((benzyloxy)methyl)-5,5-difluorotetrahydro-2H-pyran-2-ol (5.0 g, 6.23 mmol, 1.00 equiv) in DCM/MeCN (100 mL) with Et₃SiH (1.45 g, 12.47 mmol, 2.00 equiv) was added BF₃.Et₂O (1.77 g, 12.46 mmol, 2.00 equiv) dropwise at −78° C. The reaction mixture was stirred for 1 h at 0° C. Sodium bicarbonate/H₂O was added and the mixture was extracted with dichloromethane thrice. The combined extracts were washed with brine and dried over Na₂SO₄. The mixture was concentrated and the resulting residue purified by chromatography on silica gel (5:1 PE/EA) to yield (2R,4R,5S,6S)-4,5-bis(benzyloxy)-6-(2-(benzyloxy)-4-methoxy-5-(4-methoxybenzyl)phenyl)-2-((benzyloxy)methyl)-3,3-difluorotetrahydro-2H-pyran as a yellow oil.

To a mixture of (2R,4R,5S,6S)-4,5-bis(benzyloxy)-6-(2-(benzyloxy)-4-methoxy-5-(4-methoxybenzyl)phenyl)-2-((benzyloxy)methyl)-3,3-difluorotetrahydro-2H-pyran (3.8 g, 4.83 mmol, 1.00 equiv) in EA/MeOH (3:1, 50 ml) was added Pd(OH)₂/C (3.8 g), then H₂(g) was introduced into. The reaction mixture was stirred for 1 h at room temperature. The solids were filtered out. The filtrate was concentrated and the resulting residue purified by chromatograph on a C18 reversed phase column to yield (2S,3R,4R,6R)-5,5-difluoro-2-(2-hydroxy-4-methoxy-5-(4-methoxybenzyl)phenyl)-6-(hydroxymethyl)tetrahydro-2H-pyran-3,4-diol, the title compound, as a white solid.

¹H-NMR (300 MHz, CD₃OD) δ: 7.05-7.09 (m, 3H), 6.78 (d, J=5.1 Hz, 2H), 6.45 (s, 1H), 4.65 (d, J=9.2 Hz, 1H), 3.94-3.68 (m, 13H). MS(ES) m/z 425.1[M−H]⁻

### Example 11: Compound #116

### (2S,3R,4R,6R)-2-(4-chloro-5-{[4-(fluoromethoxy)phenyl]methyl}-2-hydroxyphenyl)-5,5-difluoro-6-(hydroxymethyl)oxane-3,4-diol

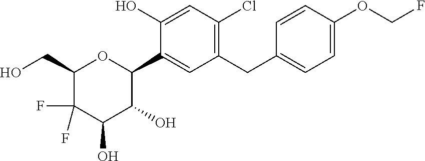 [SMILES: OC[C@H]1O[C@@H](c2cc(Cc3ccc(OCF)cc3)c(Cl)cc2O)[C@H](O)[C@@H](O)C1(F)F]

To a mixture of 2-chloro-4-hydroxybenzaldehyde (50 g, 319.35 mmol, 1.00 equiv) in dichloromethane (1.2 L) was added dibromane pyridine hydrobromide (112.8 g, 352.70 mmol, 1.10 equiv) at 0° C. The reaction mixture was stirred for 5 h at 0° C. H₂O was added and the mixture was extracted with EtOAc thrice. The combined extracts were washed with brine and dried over Na₂SO₄. The mixture was concentrated to yield 5-bromo-2-chloro-4-hydroxybenzaldehyde as a yellow solid.

To a mixture of 5-bromo-2-chloro-4-hydroxybenzaldehyde (76 g) in CH₃CN (1500 mL) with potassium carbonate (89.6 g, 648.29 mmol, 2.00 equiv) was added BnBr (83.3 g, 487.05 mmol, 1.50 equiv) dropwise. The reaction was heated to reflux for 2 h. H₂O was added and the mixture was extracted with EtOAc thrice. The combined extracts were washed with brine and dried over Na₂SO₄. The mixture was concentrated and the resulting residue purified by chromatography on silica gel (1:10 EA/PE) to yield 4-(benzyloxy)-5-bromo-2-chlorobenzaldehyde as a white solid. ¹H NMR (300 MHz, DMSO) δ: 10.1 (s, 1H), 8.03 (s, 1H), 7.35-7.51 (m, 6H), 5.39 (s, 2H).

To a mixture of 4-(benzyloxy)-5-bromo-2-chlorobenzaldehyde (20 g, 61.43 mmol, 1.00 equiv) in benzene (200 mL) with TsOH (1.1 g, 6.39 mmol, 0.10 equiv) was added ethane-1,2-diol (19.1 g, 307.73 mmol, 5.00 equiv) dropwise. The reaction was heated at 110° C. overnight. NaHCO₃/H₂O was added and the mixture extracted with ethyl acetate thrice. The combined extracts were washed with brine and dried over Na₂SO₄. The mixture was concentrated and the resulting residue purified by chromatography on silica gel (1:10 EA/PE) to yield 2-(4-(benzyloxy)-5-bromo-2-chlorophenyl)-1,3-dioxolane as a white solid. ¹H NMR (400 MHz, DMSO) δ: 7.71 (s, 1H), 7.40-7.53 (m, 4H), 7.34-7.37 (m, 2H), 5.93 (s, 1H), 5.38 (s, 2H), 4.04-4.12 (m, 2H), 3.92-4.02 (m, 2H).

To a mixture of 2-(4-(benzyloxy)-5-bromo-2-chlorophenyl)-1,3-dioxolane (6.96 g, 18.83 mmol, 1.20 equiv) in tetrahydrofuran/Toluene (20/40 mL) was added n-BuLi (2.5M in hexane, 7.3 mL, 1.15 equiv) dropwise at −78° C., and the mixture was stirred for 15 mins at −78° C. (3R,4S,6S)-3,4-Bis(benzyloxy)-6-[(benzyloxy)methyl]-5-methylideneoxan-2-one (7 g, 15.75 mmol, 1.00 equiv) in tetrahydrofuran (10 mL) was then added to the solution. The reaction mixture was stirred at −78° C. for 2 h. NH₄Cl/H₂O was added and the mixture was extracted with EtOAc thrice. The combined extracts were washed with brine and dried over Na₂SO₄. The mixture was concentrated and the resulting residue purified by chromatography on silica gel (1:5 EA/PE) to yield (3R,4S,6S)-3,4-bis(benzyloxy)-2-(2-(benzyloxy)-4-chloro-5-(1,3-dioxolan-2-yl)phenyl)-6-((benzyloxy)methyl)-5-methylenetetrahydro-2H-pyran-2-ol as a yellow oil.

To a mixture of (3R,4S,6S)-3,4-bis(benzyloxy)-2-(2-(benzyloxy)-4-chloro-5-(1,3-dioxolan-2-yl)phenyl)-6-((benzyloxy)methyl)-5-methylenetetrahydro-2H-pyran-2-ol (10.6 g, 14.42 mmol, 1.00 equiv) in tetrahydrofuran (50 mL) with 2N hydrogen chloride (8 ml) was added. The reaction mixture was stirred for 1 h at room temperature, Extracted with EtOAc thrice. The combined extracts were washed with brine and dried over Na₂SO₄. The mixture was concentrated and the resulting residue purified by chromatography on silica gel (1:10 EA/PE) to yield 4-(benzyloxy)-5-((3R,4S,6S)-3,4-bis(benzyloxy)-6-((benzyloxy)methyl)-2-hydroxy-5-methylenetetrahydro-2H-pyran-2-yl)-2-chlorobenzaldehyde as a white solid. MS (ES) m/z: 713.2[M+Na]⁺

To a mixture of 1-bromo-4-(fluoromethoxy)benzene (1.8 g, 8.78 mmol, 4.0 equiv) in tetrahydrofuran/Toluene (10/20 mL) was added n-BuLi (2.5M in hexane, 3.5 mL, 4.0 equiv) dropwise at −78° C., the mixture was stirred for 20 mins at −78° C. 4-(Benzyloxy)-5-((3R,4S,6S)-3,4-bis(benzyloxy)-6-((benzyloxy)methyl)-2-hydroxy-5-methylenetetrahydro-2H-pyran-2-yl)-2-chlorobenzaldehyde (1.5 g, 2.17 mmol, 1.00 equiv) in tetrahydrofuran (5 mL) was then added to the solution. The reaction mixture was stirred at −78° C. for 1h. NH₄Cl/H₂O was added and the mixture was extracted with EtOAc thrice. The combined extracts were washed with brine and dried over Na₂SO₄. The mixture was concentrated and the resulting residue purified by chromatography on silica gel (1:3 EA/PE) to yield (3R,4S,6S)-3,4-bis(benzyloxy)-2-(2-(benzyloxy)-4-chloro-5-((4-(fluoromethoxy)phenyl)(hydroxy)methyl)phenyl)-6-((benzyloxy)methyl)-5-methylenetetrahydro-2H-pyran-2-ol as a yellow oil. MS (ES) m/z: 807.4[M+Na]⁺

To a mixture of (3R,4S,6S)-3,4-bis(benzyloxy)-2-(2-(benzyloxy)-4-chloro-5-((4-(fluoromethoxy)phenyl)(hydroxy)methyl)phenyl)-6-((benzyloxy)methyl)-5-methylenetetrahydro-2H-pyran-2-ol (1.1 g, 1.35 mmol, 1.0 equiv) in dichloromethane (20 mL) with Et₃SiH (625 mg, 5.37 mmol, 4.00 equiv) was added TMSOTf (898 mg, 4.05 mmol, 3.00 equiv) dropwise at −78° C. The reaction mixture was stirred for 1 h at −78° C. Sodium bicarbonate/H₂O was added and the mixture was extracted with DCM thrice. The combined extracts were washed with brine and dried over Na₂SO₄. The mixture was concentrated and the resulting residue purified by chromatography on silica gel (1:10 EA/PE) to yield (2S,3R,4S,6S)-3,4-bis(benzyloxy)-2-(2-(benzyloxy)-4-chloro-5-(4-(fluoromethoxy)benzyl)phenyl)-6-((benzyloxy)methyl)-5-methylenetetrahydro-2H-pyran as a yellow oil.

To a mixture of (2S,3R,4S,6S)-3,4-bis(benzyloxy)-2-(2-(benzyloxy)-4-chloro-5-(4-(fluoromethoxy)benzyl)phenyl)-6-((benzyloxy)methyl)-5-methylenetetrahydro-2H-pyran (700 mg, 0.89 mmol, 1.00 equiv) in acetone/H₂O (20/2 mL) with NMO (313 mg, 2.67 mmol, 3.00 equiv) was added OsO₄ (1 mL, 0.25) dropwise at room temperature. The reaction mixture was stirred at 45° C. overnight. Na₂S₂O₃/H₂O was added and the mixture was extracted with ethyl acetate thrice. The combined extracts were washed with brine and dried over Na₂SO₄. The mixture was concentrated and the resulting residue purified by chromatography on silica gel (1:5 EA/PE) to yield (2R,4R,5S,6S)-4,5-bis(benzyloxy)-6-(2-(benzyloxy)-4-chloro-5-(4-(fluoromethoxy)benzyl)phenyl)-2-((benzyloxy)methyl)-3-(hydroxymethyl)tetrahydro-2H-pyran-3-ol as a yellow solid. MS (ES) m/z: 864.2[M+NH₄]⁺

To a mixture of (2R,4R,5S,6S)-4,5-bis(benzyloxy)-6-(2-(benzyloxy)-4-chloro-5-(4-(fluoromethoxy)benzyl)phenyl)-2-((benzyloxy)methyl)-3-(hydroxymethyl)tetrahydro-2H-pyran-3-ol (600 mg, 0.73 mmol, 1.00 equiv) in dichloromethane (18 mL) was added PhI(OAc)₂ (425.5 mg, 1.47 mmol, 2.00 equiv). The reaction mixture was stirred overnight at room temperature. The mixture was concentrated and the resulting residue purified by chromatography on silica gel (1:10 EA/PE) to yield (2R,4R,5S,6S)-4,5-bis(benzyloxy)-6-(2-(benzyloxy)-4-chloro-5-(4-(fluoromethoxy)benzyl)phenyl)-2-((benzyloxy)methyl)dihydro-2H-pyran-3(4H)-one as a yellow oil. MS (ES) m/z: 804.2[M+NH₄]⁺

To a mixture of (2R,4R,5S,6S)-4,5-bis(benzyloxy)-6-(2-(benzyloxy)-4-chloro-5-(4-(fluoromethoxy)benzyl)phenyl)-2-((benzyloxy)methyl)dihydro-2H-pyran-3(4H)-one (180 mg, 0.23 mmol, 1.00 equiv) in dichloromethane (3 mL) was added DAST (0.12 mL, 4.00 equiv) dropwise at 0° C. The reaction mixture was stirred overnight at room temperature. Sodium bicarbonate/H₂O was added and the mixture was extracted with dichloromethane thrice. The combined extracts were washed with brine and dried over Na₂SO₄. The mixture was concentrated and the resulting residue purified by chromatography on silica gel (1:10 EA/PE) to yield (2R,4R,5S,6S)-4,5-bis(benzyloxy)-6-(2-(benzyloxy)-4-chloro-5-(4-(fluoromethoxy)benzyl)phenyl)-2-((benzyloxy)methyl)-3,3-difluorotetrahydro-2H-pyran as a yellow oil. MS (ES) m/z: 831.6[M+Na]⁺.

To a mixture of (2R,4R,5S,6S)-4,5-bis(benzyloxy)-6-(2-(benzyloxy)-4-chloro-5-(4-(fluoromethoxy)benzyl)phenyl)-2-((benzyloxy)methyl)-3,3-difluorotetrahydro-2H-pyran (130 mg, 0.16 mmol, 1.00 equiv) in EA/MeOH (3:1, 3 mL) was added Pd(OH)₂/C (50 mg) at room temperature, and hydrogen was introduced. The reaction mixture was stirred for 20 min at room temperature. The solids were filtered out. The filtrate was concentrated and the resulting residue purified by chromatograph on a C18 reversed phase column to yield (2S,3R,4R,6R)-2-(4-chloro-5-(4-(fluoromethoxy)benzyl)-2-hydroxyphenyl)-5,5-difluoro-6-(hydroxymethyl)tetrahydro-2H-pyran-3,4-diol, the title compound, as a white solid.

¹H-NMR (MeOD, 300 MHz) δ: 7.25 (s, 1H), 7.15 (d, J=8.7 Hz, 2H), 6.98 (d, J=8.7 Hz, 2H), 6.87 (s, 1H), 5.79 (s, 1H), 6.61 (s, 1H), 4.71 (d, J=9.9 Hz, 1H), 3.98 (s, 2H), 3.71-3.83 (m, 5H). MS (ES) m/z: 447.0[M−H]⁻

### Example 12: Compound #127

### (2S,3R,4R,6R)-2-{4-chloro-5-[(4-ethylphenyl)methyl]-2-hydroxyphenyl}-5,5-difluoro-6-(hydroxymethyl)oxane-3,4-diol

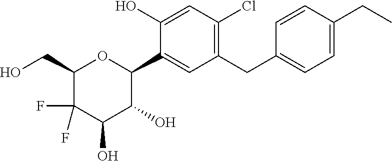 [SMILES: CCc1ccc(Cc2cc([C@@H]3O[C@H](CO)C(F)(F)[C@H](O)[C@H]3O)c(O)cc2Cl)cc1]

To a mixture of 1-bromo-4-ethylbenzene (1.1 g, 5.94 mmol, 1.20 equiv) in tetrahydrofuran ((20 mL) was added n-BuLi (2.5M in hexane, 2.0 mL, 1.10 equiv) dropwise at −78° C., the mixture was stirred for 30 mins at −78° C. 4-(Benzyloxy)-5-bromo-2-chlorobenzaldehyde (1.5 g, 4.61 mmol, 1.00 equiv) in tetrahydrofuran (10 mL) was then added to the solution. The reaction mixture was stirred at −78° C. for 2h. NH₄Cl/H₂O was added and the mixture was extracted with EtOAc thrice. The combined extracts were washed with brine and dried over Na₂SO₄. The mixture was concentrated and the resulting residue purified by chromatography on silica gel (5% EA/PE) to yield (4-(benzyloxy)-5-bromo-2-chlorophenyl)(4-ethylphenyl)methanol as a yellow oil. MS (ES) m/z: 414.9[M-OH]⁺

To a mixture of (4-(benzyloxy)-5-bromo-2-chlorophenyl)(4-ethylphenyl)methanol (840 mg, 1.95 mmol, 1.00 equiv) in dichloromethane (8 mL) with Et₃SiH (451 mg, 3.88 mmol, 2.00 equiv) was added CF₃COOH (332 mg, 2.94 mmol, 1.50 equiv) dropwise at 0° C. The reaction mixture was stirred for 1 h at 0° C. Sodium bicarbonate/H₂O was added and the mixture was extracted with DCM thrice. The combined extracts were washed with brine and dried over Na₂SO₄. The mixture was concentrated and the resulting residue purified by chromatography on silica gel (1:20 EA/PE) to yield 1-(benzyloxy)-2-bromo-5-chloro-4-(4-ethylbenzyl)benzene as a yellow solid. HNMR (300 MHz, Chloroform-d) δ: 7.26-7.52 (m, 6H), 7.12 (q, J=8.1 Hz, 4H), 6.98 (s, 1H), 5.13 (s, 2H), 3.98 (s, 2H), 2.64 (q, J=7.6 Hz, 2H), 1.24 (t, J=7.6 Hz, 3H).

To a mixture of 1-(benzyloxy)-2-bromo-5-chloro-4-(4-ethylbenzyl)benzene (540 mg, 1.30 mmol, 1.20 equiv) in tetrahydrofuran/toluene (3/6 mL) was added n-BuLi (2.5M in hexane, 0.52 mL, 1.20 equiv) dropwise at −78° C., the mixture was stirred at −78° C. for 30 min. (3R,4R,6R)-3,4-Bis(benzyloxy)-6-[(benzyloxy)methyl]-5,5-difluorooxan-2-one (510 mg, 1.09 mmol, 1.00 equiv) in tetrahydrofuran (1 mL) was then added to the solution. The reaction mixture was stirred at −78° C. for 2h. NH₄Cl/H₂O was added and the mixture was extracted with EtOAc thrice. The combined extracts were washed with brine and dried over Na₂SO₄. The mixture was concentrated and the resulting residue purified by chromatography on silica gel (1:5 EA/PE) to yield (3R,4R,6R)-3,4-bis(benzyloxy)-2-(2-(benzyloxy)-4-chloro-5-(4-ethylbenzyl)phenyl)-6-((benzyloxy)methyl)-5,5-difluorotetrahydro-2H-pyran-2-ol as a yellow oil. MS (ES) m/z: 787.3[M-OH]⁺

To a mixture of (3R,4R,6R)-3,4-bis(benzyloxy)-2-(2-(benzyloxy)-4-chloro-5-(4-ethylbenzyl)phenyl)-6-((benzyloxy)methyl)-5,5-difluorotetrahydro-2H-pyran-2-ol (700 mg, 0.87 mmol, 1.00 equiv) in dichloromethane (10 mL) with Et₃SiH (202 mg, 1.74 mmol, 2.00 equiv) was added BF₃.Et₂O (185 mg, 1.30 mmol, 1.50 equiv) at 0° C. The reaction mixture was stirred at 0° C. for 1h. Sodium bicarbonate/H₂O was added and the mixture was extracted with dichloromethane thrice. The combined extracts were washed with brine and dried over Na₂SO₄. The mixture was concentrated and the resulting residue purified by chromatography on silica gel (1:10 EA/PE) to yield (2R,4R,5S,6S)-4,5-bis(benzyloxy)-6-(2-(benzyloxy)-4-chloro-5-(4-ethylbenzyl)phenyl)-2-((benzyloxy)methyl)-3,3-difluorotetrahydro-2H-pyran as a light yellow oil. MS (ES) m/z: 811.2[M+Na⁺]

To a mixture of (2R,4R,5S,6S)-4,5-bis(benzyloxy)-6-(2-(benzyloxy)-4-chloro-5-(4-ethylbenzyl)phenyl)-2-((benzyloxy)methyl)-3,3-difluorotetrahydro-2H-pyran (550 mg, 0.70 mmol, 1.00 equiv) in EA/MeOH (3:1, 10 mL) was added Pd(OH)₂/C (100 mg) at room temperature, and hydrogen was introduced. The reaction mixture was stirred for 20 min at room temperature. The solids were filtered out. The filtrate was concentrated and the resulting residue purified by chromatograph on a C18 reversed phase column to yield (2S,3R,4R,6R)-2-(4-chloro-5-(4-ethylbenzyl)-2-hydroxyphenyl)-5,5-difluoro-6-(hydroxymethyl)tetrahydro-2H-pyran-3,4-diol, the title compound, as a white solid.

¹H-NMR (MeOD, 300 MHz) δ: 7.23 (s, 1H), 7.08 (s, 4H), 6.87 (s, 1H), 4.70 (d, J=9.0 Hz, 1H), 3.71-3.97 (m, 7H), 2.59 (d, J=7.2 Hz, 2H), 1.20 (t, J=6.9 Hz, 3H). MS (ES) m/z: 427.0[M−H]⁻

### Example 13: Compound #131

### (2S,3R,4R,6R)-2-(5-(4-ethylbenzyl)-2-hydroxy-4-methylphenyl)-5,5-difluoro-6-(hydroxymethyl)tetrahydro-2H-pyran-3,4-diol

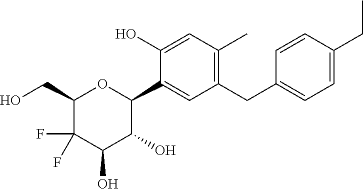 [SMILES: CCc1ccc(Cc2cc([C@@H]3O[C@H](CO)C(F)(F)[C@H](O)[C@H]3O)c(O)cc2C)cc1]

To a mixture of 1-bromo-4-ethylbenzene (1.5 g, 8.11 mmol, 1.10 equiv) in THE (15 mL) was added n-BuLi (2.5M in hexane, 3.40 mL, 1.15 equiv) dropwise at −78° C., the mixture was stirred at −78° C. for 30 min. 4-(Benzyloxy)-5-bromo-2-methylbenzaldehyde (2.25 g, 7.37 mmol, 1.00 equiv) in THE (5 mL) was then added to the solution. The reaction mixture was stirred at −78° C. for 2h. NH₄Cl/H₂O was added and the mixture was extracted with EtOAc thrice. The combined extracts were washed with brine and dried over Na₂SO₄. The mixture was then concentrated to yield (4-(benzyloxy)-5-bromo-2-methylphenyl)(4-ethylphenyl)methanol as a light yellow oil.

To a mixture of (4-(benzyloxy)-5-bromo-2-methylphenyl)(4-ethylphenyl)methanol (3.22 g) in DCM (50 mL) with Et₃SiH (2.73 g, 23.48 mmol, 3.00 equiv) was added BF₃.Et₂O (2.23 g, 15.70 mmol, 2.01 equiv) at 0° C. The reaction mixture was stirred at 0° C. for 2h. Sodium bicarbonate/H₂O was added and the mixture was extracted with dichloromethane thrice. The combined extracts were washed with brine and dried over Na₂SO₄. The mixture was concentrated and the resulting residue purified by chromatography on silica gel (5:1 PE/EA) to yield 1-(benzyloxy)-2-bromo-4-(4-ethylbenzyl)-5-methylbenzene as a light yellow oil. 1H NMR (300 MHz, Chloroform-d) δ: 7.46-7.54 (m, 2H), 7.25-7.45 (m, 4H), 7.09-7.16 (m, 2H), 6.98-7.06 (m, 2H), 6.78 (s, 1H), 5.14 (s, 2H), 3.87 (s, 2H), 2.63 (q, J=7.6 Hz, 2H), 2.19 (s, 3H), 1.23 (t, J=7.7 Hz, 3H).

To a mixture of 1-(benzyloxy)-2-bromo-4-(4-ethylbenzyl)-5-methylbenzene (328 mg, 0.83 mmol, 1.30 equiv) in THE (5 mL) was added n-BuLi (2.5M in hexane, 0.33 mL, 1.29 equiv) dropwise at −78° C., and the mixture was stirred at −78° C. for 30 min. (3R,4R,6R)-3,4-Bis(benzyloxy)-6-[(benzyloxy)methyl]-5,5-difluorooxan-2-one (300 mg, 0.64 mmol, 1.00 equiv) in THE (1 mL) was then added to the solution. The reaction was stirred at −78° C. for 2h. NH₄Cl/H₂O was added and the mixture was extracted with EtOAc thrice. The combined extracts were washed with brine and dried over Na₂SO₄. The mixture was concentrated and the resulting residue purified by chromatography on silica gel (3:1 PE/EA) to yield (3R,4R,6R)-3,4-bis(benzyloxy)-2-(2-(benzyloxy)-5-(4-ethylbenzyl)-4-methylphenyl)-6-((benzyloxy)methyl)-5,5-difluorotetrahydro-2H-pyran-2-ol as a light yellow oil. MS (ES) m/z: 767.5 [M-OH]⁺

To a mixture of (3R,4R,6R)-3,4-bis(benzyloxy)-2-(2-(benzyloxy)-5-(4-ethylbenzyl)-4-methylphenyl)-6-((benzyloxy)methyl)-5,5-difluorotetrahydro-2H-pyran-2-ol (420 mg, 0.54 mmol, 1.00 equiv) in DCM (10 mL) with Et₃SiH (186.4 mg, 1.60 mmol, 3.00 equiv) was added BF₃.Et₂O (152 mg, 1.07 mmol, 2.00 equiv) at 0° C. The reaction mixture was stirred at 0° C. for 1h. Sodium bicarbonate/H₂O was added and the mixture was extracted with dichloromethane thrice. The combined extracts were washed with brine and dried over Na₂SO₄. The mixture was concentrated and the resulting residue purified by chromatography on silica gel (5:1 PE/EA) to yield (2R,4R,5S,6S)-4,5-bis(benzyloxy)-6-(2-(benzyloxy)-5-(4-ethylbenzyl)-4-methylphenyl)-2-((benzyloxy)methyl)-3,3-difluorotetrahydro-2H-pyran as a light yellow oil. MS (ES) m/z: 791.6 [M+Na]⁺

To a solution of (2R,4R,5S,6S)-4,5-bis(benzyloxy)-6-(2-(benzyloxy)-5-(4-ethylbenzyl)-4-methylphenyl)-2-((benzyloxy)methyl)-3,3-difluorotetrahydro-2H-pyran (210 mg, 0.27 mmol, 1.00 equiv) in EA/MeOH (3:1) (5 mL) was added Pd(OH)₂/C (120 mg). H₂ was then introduced. The reaction mixture was stirred for 30 min at room temperature. The solids were filtered out. The mixture was concentrated and the resulting residue purified by chromatography on C18 (5%-50% CH3CN/H₂O) to yield (2S,3R,4R,6R)-2-(5-(4-ethylbenzyl)-2-hydroxy-4-methylphenyl)-5,5-difluoro-6-(hydroxymethyl)tetrahydro-2H-pyran-3,4-diol, the title compound, as a white solid.

¹H NMR (300 MHz, CD₃OD) δ: 7.13 (s, 1H), 6.98-7.11 (m, 4H), 6.66 (s, 1H), 4.71 (d, J=9.2 Hz, 1H), 3.70-3.96 (m, 7H), 2.60 (q, J=7.6 Hz, 2H), 2.11 (s, 3H), 1.21 (t, J=7.6 Hz, 3H). MS (ES) m/z: 407.1 [M−H]⁻.

### Example 14: Compound #124

### (2S,3R,4R,6R)-2-(5-(4-cyclopropylbenzyl)-2-hydroxy-4-methylphenyl)-5,5-difluoro-6-(hydroxymethyl)-tetrahydro-2H-pyran-3,4-diol

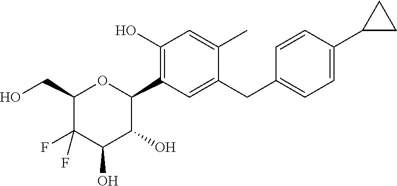 [SMILES: Cc1cc(O)c([C@@H]2O[C@H](CO)C(F)(F)[C@H](O)[C@H]2O)cc1Cc1ccc(C2CC2)cc1]

To a mixture of 1-bromo-4-cyclopropylbenzene (2.200 g, 11.16 mmol, 1.00 equiv) in THE (50 mL) was added n-BuLi (2.5 M in hexane, 4.9 mL, 12.25 mmol, 1.10 equiv) dropwise at −78° C. under N₂. The solution was stirred for 15 min at −78° C. To the mixture was then added a solution of 1-bromo-4-cyclopropylbenzene (3.100 g, 10.16 mmol, 0.91 equiv) in THE (5 mL) dropwise at −78° C. The reaction mixture was stirred for 2 h at −78° C. NH₄Cl/H₂O was added and the mixture was extracted with EtOAc thrice. The combined extracts were concentrated and chromatograph on silica gel (20:1 PE/EA) to yield (4-(benzyloxy)-5-bromo-2-methylphenyl)(4-cyclopropylphenyl)methanol as a colorless oil.

To a mixture of (4-(benzyloxy)-5-bromo-2-methylphenyl)(4-cyclopropylphenyl)methanol (4.000 g, 9.45 mmol, 1.00 equiv) in dichloromethane (20 mL) with Et₃SiH (3.0 mL) was added trifluoroacetic acid (1.0 mL). The reaction mixture was stirred for 1 h at 0° C. Sodium bicarbonate (aq.) was added and the mixture was extracted with DCM thrice. The combined extracts were concentrated and chromatograph on silica gel (10:1 PE/EA) to yield 1-(benzyloxy)-2-bromo-4-(4-cyclopropylbenzyl)-5-methylbenzene as a white solid.

To a mixture of 1-(benzyloxy)-2-bromo-4-(4-cyclopropylbenzyl)-5-methylbenzene (0.600 g, 1.47 mmol, 1.00 equiv) in THE (12 mL) and toluene (24.0 mL) was added n-BuLi (2.5 M in hexane, 0.6 mL, 1.5 mmol, 1.02 equiv) dropwise at −78° C. under N₂. The solution was stirred for 15 min at −78° C. To the mixture was then added a solution of (3R,4S,6S)-3,4-bis(benzyloxy)-6-[(benzyloxy)methyl]-5-methylideneoxan-2-one (0.600 g, 1.35 mmol, 0.92 equiv) in tetrahydrofuran (5.0 mL) dropwise at −78° C. The reaction mixture was stirred for 2 h at −78° C. NH₄Cl/H₂O was added and the mixture was extracted with EtOAc thrice. The combined extracts were concentrated and chromatograph on silica gel (10:1 PE/EA) to yield (3R,4S,6S)-3,4-bis(benzyloxy)-2-(2-(benzyloxy)-5-(4-cyclopropylbenzyl)-4-methylphenyl)-6-((benzyloxy)methyl)-5-methylenetetrahydro-2H-pyran-2-ol as a colorless oil. MS (ES) m/z: 790 [M+NH₄]⁺.

To a mixture of (3R,4S,6S)-3,4-bis(benzyloxy)-2-(2-(benzyloxy)-5-(4-cyclopropylbenzyl)-4-methylphenyl)-6-((benzyloxy)methyl)-5-methylenetetrahydro-2H-pyran-2-ol (0.600 g, 0.78 mmol, 1.00 equiv) in dichloromethane (10 mL) with Et₃SiH (0.250 g, 2.15 mmol, 2.77 equiv) was added boron fluoride ethyl ether (0.300 g, 2.12 mmol, 2.72 equiv). The reaction mixture was stirred for 1 h at 0° C. Sodium bicarbonate (aq.) was added and the mixture was extracted with EA thrice. The combined extracts were concentrated and chromatograph on silica gel (10:1 PE/EA) to yield (2S,3R,4S,6S)-3,4-bis(benzyloxy)-2-(2-(benzyloxy)-5-(4-cyclopropylbenzyl)-4-methylphenyl)-6-((benzyloxy)methyl)-5-methylenetetrahydro-2H-pyran as a colorless oil. MS (ES) m/z: 774 [M+NH₄]⁺.

To a mixture of (2S,3R,4S,6S)-3,4-bis(benzyloxy)-2-(2-(benzyloxy)-5-(4-cyclopropylbenzyl)-4-methylphenyl)-6-((benzyloxy)methyl)-5-methylenetetrahydro-2H-pyran (0.450 g, 0.59 mmol, 1.00 equiv) in acetone (20 mL) with NMO (0.225 g, 1.92 mmol, 3.23 equiv) was added OsO₄ (25%, in t-BuOH, 0.6 mL). The reaction mixture was stirred for 16 h at 25° C. Sodium thiosulfate (aq.) was added and the mixture was extracted with EA thrice. The combined extracts were concentrated and chromatograph on silica gel (3:1 PE/EA) to yield (2R,4R,5S,6S)-4,5-bis(benzyloxy)-6-(2-(benzyloxy)-5-(4-cyclopropylbenzyl)-4-methylphenyl)-2-((benzyloxy)methyl)-3-(hydroxymethyl)tetrahydro-2H-pyran-3-ol as a colorless oil. MS (ES) m/z: 808 [M+NH₄]⁺.

To a mixture of (2R,4R,5S,6S)-4,5-bis(benzyloxy)-6-(2-(benzyloxy)-5-(4-cyclopropylbenzyl)-4-methylphenyl)-2-((benzyloxy)methyl)-3-(hydroxymethyl)tetrahydro-2H-pyran-3-ol (0.340 g, 0.43 mmol, 1.00 equiv) in DCM (5 mL) was added PhI(OAc)₂ (0.280 g, 0.87 mmol, 2.55 equiv). The reaction mixture was stirred for 16 h at 25° C. Water was added and the mixture was extracted with EA thrice. The combined extracts were concentrated and chromatograph on silica gel (10:1 PE/EA) to yield (2R,4R,5S,6S)-4,5-bis(benzyloxy)-6-(2-(benzyloxy)-5-(4-cyclopropylbenzyl)-4-methylphenyl)-2-((benzyloxy)methyl)dihydro-2H-pyran-3(4H)-one as a colorless oil. MS (ES) m/z: 776 (M+NH₄)⁺.

To a mixture of (2R,4R,5S,6S)-4,5-bis(benzyloxy)-6-(2-(benzyloxy)-5-(4-cyclopropylbenzyl)-4-methylphenyl)-2-((benzyloxy)methyl)dihydro-2H-pyran-3(4H)-one (0.300 g, 0.40 mmol, 1.00 equiv) in DCM (10 mL) was added DAST (0.300 g, 1.86 mmol, 4.71 equiv). The reaction mixture was stirred for 16 h at 25° C. Sodium bicarbonate (aq.) was added and the mixture was extracted with EA thrice. The combined extracts were concentrated and chromatograph on silica gel (10:1 PE/EA) to yield (2R,4R,5S,6S)-4,5-bis(benzyloxy)-6-(2-(benzyloxy)-5-(4-cyclopropylbenzyl)-4-methylphenyl)-2-((benzyloxy)methyl)-3,3-difluorotetrahydro-2H-pyran as a colorless oil. MS (ES) m/z: 798 [M+NH₄]⁺.

To a mixture of (2R,4R,5S,6S)-4,5-bis(benzyloxy)-6-(2-(benzyloxy)-5-(4-cyclopropylbenzyl)-4-methylphenyl)-2-((benzyloxy)methyl)-3,3-difluorotetrahydro-2H-pyran (0.250 g, 0.32 mmol, 1.00 equiv) in dichloromethane (10 mL) with 1,2,3,4,5-pentamethylbenzene (0.500 g, 3.37 mmol, 10.54 equiv) was added BCl₃ (1M, 5.0 mL, 5 mmol, 15.63 equiv) dropwise at −78° C. The reaction mixture was stirred for 1 h at −78° C. Methanol was added. The mixture was concentrated and the resulting residue purified by chromatography on C18 (10%-50% CH₃CN/H₂O) g to yield (2S,3R,4R,6R)-2-(5-(4-cyclopropylbenzyl)-2-hydroxy-4-methylphenyl)-5,5-difluoro-6-(hydroxymethyl)tetrahydro-2H-pyran-3,4-diol, the title compound, as a white solid.

¹H NMR (300 MHz, Methanol-d₄) δ: 7.13 (s, 1H), 6.97 (q, J=8.2 Hz, 4H), 6.65 (s, 1H), 4.71 (d, J=9.1 Hz, 1H), 3.72-3.97 (m, 7H), 2.11 (s, 3H), 1.86 (m, 1H), 0.85-0.97 (m, 2H), 0.58-0.70 (m, 2H); MS (ES) m/z: 419 [M−H]⁻.

### Example 15: Compound #171

### (2S,3R,4R,6R)-2-(4-chloro-2-hydroxy-5-(4-methylbenzyl)phenyl)-5,5-difluoro-6-(hydroxymethyl)-tetrahydro-2H-pyran-3,4-diol

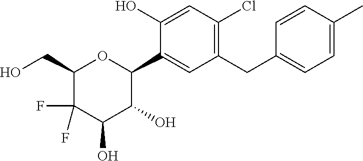 [SMILES: Cc1ccc(Cc2cc([C@@H]3O[C@H](CO)C(F)(F)[C@H](O)[C@H]3O)c(O)cc2Cl)cc1]

To a mixture of 1-bromo-4-methylbenzene (527 mg, 3.08 mmol, 1.00 equiv) in tetrahydrofuran (10 mL) was added n-BuLi (1.6M in hexane, 1.93 mL, 3.08 mmol, 1.00 equiv) dropwise at −78° C., and the mixture was stirred for 30 mins at −78° C. 4-(Benzyloxy)-5-bromo-2-chlorobenzaldehyde (1 g, 3.07 mmol, 1.00 equiv) in tetrahydrofuran (4 mL) was added to the solution. The reaction mixture was stirred at −78° C. for 2h. Ice/water was added and the mixture was extracted with EtOAc twice. The combined extracts were dried over Na₂SO₄. The mixture was concentrated and the resulting residue purified by chromatography on silica gel (3:1 PE/EA) to yield (4-(benzyloxy)-5-bromo-2-chlorophenyl)(p-tolyl)methanol as a yellow oil.

To a mixture of (4-(benzyloxy)-5-bromo-2-chlorophenyl)(p-tolyl)methanol (1.1 g, 2.63 mmol, 1.00 equiv) in dichloromethane (15 mL) with Et₃SiH (930 mg, 8.00 mmol, 3.00 equiv) was added BF₃.Et₂O (747 mg, 5.26 mmol, 2.00 equiv) dropwise at 0° C. The reaction mixture was stirred for 1 h at 0° C. Water/ice was added and the mixture was extracted with DCM twice. The combined extracts were washed with water and dried over Na₂SO₄. The mixture was concentrated and the resulting residue purified by chromatography on silica gel (4:1 PE/EA) to yield 1-(benzyloxy)-2-bromo-5-chloro-4-(4-methylbenzyl)benzene as a yellow oil. 1H-NMR (300 MHz, CDCl₃) δ: 7.23-7.49 (m, 6H), 7.08 (q, J=8.2 Hz, 4H), 6.96 (s, 1H), 5.10 (s, 2H), 3.95 (s, 2H), 2.32 (s, 3H).

To a mixture of 1-(benzyloxy)-2-bromo-5-chloro-4-(4-methylbenzyl)benzene (600 mg, 1.49 mmol, 1.00 equiv) in tetrahydrofuran (10 mL) was added n-BuLi (1.6M in hexane, 0.93 mL, 1.49 mmol, 1.00 equiv) dropwise at −78° C., and the mixture was stirred for 30 mins at −78° C. (3R,4R,6R)-3,4-Bis(benzyloxy)-6-(benzyloxymethyl)-5,5-difluoro-tetrahydropyran-2-one (663 mg, 1.41 mmol, 0.95 equiv) in tetrahydrofuran (3 mL) was then added to the solution. The reaction mixture was stirred at −78° C. for 2h. Water/ice was added and the mixture was extracted with EtOAc twice. The combined extracts were washed with brine and dried over Na₂SO₄. The mixture was concentrated and the resulting residue purified by chromatography on silica gel (2:1 PE/EA) to yield (3R,4R,6R)-3,4-bis(benzyloxy)-2-(2-(benzyloxy)-4-chloro-5-(4-methylbenzyl)phenyl)-6-((benzyloxy)methyl)-5,5-difluorotetrahydro-2H-pyran-2-ol as a yellow oil.

To a mixture of (3R,4R,6R)-3,4-bis(benzyloxy)-2-(2-(benzyloxy)-4-chloro-5-(4-methylbenzyl)phenyl)-6-((benzyloxy)methyl)-5,5-difluorotetrahydro-2H-pyran-2-ol (400 mg, 0.51 mmol, 1.00 equiv) in dichloromethane (10 mL) with Et₃SiH (176 mg, 1.52 mmol, 3.00 equiv) was added BF₃.Et₂O (144 mg, 1.01 mmol, 2.00 equiv) dropwise at 0° C. The reaction mixture was stirred at 0° C. for 1h. Water/ice was added and the mixture was extracted with DCM twice. The combined extracts were washed with water and dried over Na₂SO₄. The mixture was concentrated and the resulting residue purified by chromatography on silica gel (3:1 PE/EA) to yield (2R,4R,5S,6S)-4,5-bis(benzyloxy)-6-(2-(benzyloxy)-4-chloro-5-(4-methylbenzyl)phenyl)-2-((benzyloxy)methyl)-3,3-difluorotetrahydro-2H-pyran as a yellow oil.

To a mixture of (2R,4R,5S,6S)-4,5-bis(benzyloxy)-6-(2-(benzyloxy)-4-chloro-5-(4-methylbenzyl)phenyl)-2-((benzyloxy)methyl)-3,3-difluorotetrahydro-2H-pyran (250 mg, 0.32 mmol, 1.00 equiv) in dichloromethane (5 mL) with 1,2,3,4,5-pentamethylbenzene (448 mg, 3.22 mmol, 10.00 equiv) was added BCl₃ (1M in DCM, 3.22 mL, 3.22 mmol, 10.00 equiv) dropwise at −78° C. The reaction mixture was stirred for 1h at −78° C. Methanol (10 mL) was added to quench the reaction. The mixture was concentrated and the solids were filtered out. The resulting mixture was purified by chromatography on a C18 reversed phase column H₂O/ACN (45%-75%) to yield (2S,3R,4R,6R)-2-(4-chloro-2-hydroxy-5-(4-methylbenzyl)phenyl)-5,5-difluoro-6-(hydroxymethyl)tetrahydro-2H-pyran-3,4-diol as a white solid.

¹H-NMR (300 MHz, CD₃OD) δ: 7.24 (s, 1H), 7.07 (s, 4H), 6.88 (s, 1H), 4.72 (d, J=9.6 Hz, 1H), 3.98 (s, 2H), 3.69-3.92 (m, 5H), 2.30 (s, 3H); MS(ES) m/z: 432[M+NH₄]⁺

### Example 16: Compound #163

### (2S,3R,4R,6R)-2-(4-chloro-5-(4-chlorobenzyl)-2-hydroxyphenyl)-5,5-difluoro-6-(hydroxymethyl)-tetrahydro-2H-pyran-3,4-diol

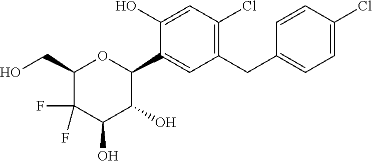 [SMILES: OC[C@H]1O[C@@H](c2cc(Cc3ccc(Cl)cc3)c(Cl)cc2O)[C@H](O)[C@@H](O)C1(F)F]

To a mixture of 1-bromo-4-chlorobenzene (0.644 g, 3.36 mmol, 1.00 equiv) in THE (20 mL) was added n-BuLi (2.5 M in hexane, 1.3 mL, 3.25 mmol, 0.97 equiv) dropwise at −78° C. The reaction mixture was stirred for 15 min at −78° C. To the mixture was then added a solution of 4-(benzyloxy)-5-bromo-2-chlorobenzaldehyde (1.000 g, 3.07 mmol, 0.91 equiv) in tetrahydrofuran (5.0 mL) dropwise at −78° C. The reaction mixture was stirred for 2 h at −78° C. NH₄Cl/H₂O was added and the mixture was extracted with EtOAc thrice. The combined extracts were concentrated and chromatograph on silica gel (50:1-10:1 PE/EA) to yield (4-(benzyloxy)-5-bromo-2-chlorophenyl)(4-chlorophenyl)methanol as a yellow oil.

To a mixture of (4-(benzyloxy)-5-bromo-2-chlorophenyl)(4-chlorophenyl)methanol (1.200 g, 2.74 mmol, 1.00 equiv) in dichloromethane (10 mL) with Et₃SiH (0.9 mL) was added trifluoroacetic acid (0.4 mL). The reaction mixture was stirred for 1 h at 0° C. Sodium bicarbonate (aq.) was added and the mixture was extracted with DCM thrice. The combined extracts were concentrated and chromatograph on silica gel (50:1-10:1 PE/EA) to yield 1-(benzyloxy)-2-bromo-5-chloro-4-(4-chlorobenzyl)benzene as a white solid.

To a mixture of 1-(benzyloxy)-2-bromo-5-chloro-4-(4-chlorobenzyl)benzene (0.300 g, 0.71 mmol, 1.00 equiv) in THE (3 mL) and toluene (6.0 mL) was added n-BuLi (2.5 M in hexane, 0.3 mL, 0.75 mmol, 1.06 equiv) dropwise at −78° C. under N₂. The solution was stirred for 15 min at −78° C. To the mixture was then added a solution of (3R, 4R, 6R)-3,4-bis(benzyloxy)-6-[(benzyloxy)methyl]-5,5-difluorooxan-2-one (0.300 g, 0.64 mmol, 0.90 equiv) in tetrahydrofuran (2 mL) dropwise at −78° C. The reaction mixture was stirred for 2 h at −78° C. NH₄Cl/H₂O was added and the mixture was extracted with EtOAc thrice. The combined extracts were concentrated and chromatograph on silica gel (50:1-10:1 PE/EA) to yield (3R,4R,6R)-3,4-bis(benzyloxy)-2-(2-(benzyloxy)-4-chloro-5-(4-chlorobenzyl)phenyl)-6-((benzyloxy)methyl)-5,5-difluorotetrahydro-2H-pyran-2-ol as a yellow oil. MS (ES) m/z: 828 [M+NH₄]⁺.

To a mixture of (3R,4R,6R)-3,4-bis(benzyloxy)-2-(2-(benzyloxy)-4-chloro-5-(4-chlorobenzyl)phenyl)-6-((benzyloxy)methyl)-5,5-difluorotetrahydro-2H-pyran-2-ol (0.380 g, 0.47 mmol, 1.00 equiv) in dichloromethane (5 mL) with Et₃SiH (0.2 mL) was added boron fluoride ethyl ether (0.1 mL). The reaction mixture was stirred for 1 h at 0° C. Sodium bicarbonate (aq.) was added and the mixture was extracted with DCM thrice. The combined extracts were concentrated and purified by chromatography on silica gel (50:1-3:1 PE/EA) to yield (2R,4R,5S,6S)-4,5-bis(benzyloxy)-6-(2-(benzyloxy)-4-chloro-5-(4-chlorobenzyl)phenyl)-2-((benzyloxy)methyl)-3,3-difluorotetrahydro-2H-pyran as a yellow oil. MS (ES) m/z: 812 [M+NH₄]⁺.

To a mixture of (2R,4R,5S,6S)-4,5-bis(benzyloxy)-6-(2-(benzyloxy)-4-chloro-5-(4-chlorobenzyl)phenyl)-2-((benzyloxy)methyl)-3,3-difluorotetrahydro-2H-pyran (0.170 g, 0.21 mmol, 1.00 equiv) in dichloromethane (5 mL) with 1,2,3,4,5-pentamethylbenzene (0.340 g, 2.29 mmol, 10.74 equiv) was added BCl₃ (1 M, 3.4 mL, 3.4 mmol, 16.19 equiv) dropwise at −78° C. The reaction mixture was stirred for 1 h at −78° C. Methanol was added. The mixture was concentrated and the resulting residue purified by chromatography on a C18 reversed phase column to yield (2S,3R,4R,6R)-2-(4-chloro-5-(4-chlorobenzyl)-2-hydroxyphenyl)-5,5-difluoro-6-(hydroxymethyl)tetrahydro-2H-pyran-3,4-diol, the title compound, as a white solid.

¹H NMR (300 MHz, Methanol-d₄) δ: 7.22-7.30 (m, 3H), 7.13-7.21 (m, 2H), 6.88 (s, 1H), 4.73 (d, J=9.7 Hz, 1H), 4.01 (d, J=2.6 Hz, 2H), 3.67-3.95 (m, 5H); MS (ES) m/z: 452 [M+NH₄]⁺.

### Example 17: Compound #31

### (2S,3R,4R,6R)-2-[5-(benzothiophen-2-ylmethyl)-2-hydroxy-4-methoxy-phenyl]-5,5-difluoro-6-(hydroxymethyl)tetrahydropyran-3,4-diol

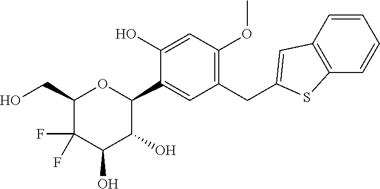 [SMILES: COc1cc(O)c([C@@H]2O[C@H](CO)C(F)(F)[C@H](O)[C@H]2O)cc1Cc1cc2ccccc2s1]

The 1.6 M of n-hexane solution of n-butyllithium (1.71 ml) was added dropwise to a solution of 2-(4-(benzyloxy)-5-bromo-2-methoxybenzyl)benzo[b]thiophene (1200 mg, 2.73 mmol) in THE (6 ml) at 78° C. under an atmosphere of argon, and the mixture was stirred for 45 min. The mixture was then transferred via cannula to a cooled solution (−78° C.) of (3R,4S,6S)-3,4-bis(benzyloxy)-6-((benzyloxy)methyl)-5-methylenetetrahydro-2H-pyran-2-one (1214 mg, 2.73 mmol) in THE (8 ml). The resulting reaction mixture was stirred for 80 min without temperature adjustment. An aqueous solution of saturated aqueous NH₄Cl solution (8 ml) was added to the resulting mixture and the organic layer was separated. The aqueous layer was extracted with EtOAc three times (35 mL each time). The combined organic extracts were washed with saturated saline solution and dried over Na₂SO₄. After filtration, the solvent was evaporated from the filtrate under reduced pressure to yield a white foam.

Without additional purification, the isolated white foam was dissolved in DCM (20 mL) and ACN (20 mL) and the resulting solution was cooled to 0° C., evacuated, and re-filled with argon. To the resulting mixture was added triethylsilane (1.44 mL), followed by dropwise addition of BF₃.Et₂O (0.69 ml). The mixture was stirring at temperature for 50 min, then quenched with saturated NaHCO₃ solution. The organic layer was separated and the aqueous layer was extracted with EtOAc three times (30 mL each). The combined organic extracts were concentrated to yield a mixture of alpha and beta-anomers, which were further purified by flash column chromatography on silica gel (80 g, EtOAc/heptane: 0>>>10%) to yield (2R,3R,4S,6S)-2-(5-(benzo[b]thiophen-2-ylmethyl)-2-(benzyloxy)-4-methoxyphenyl)-3,4-bis(benzyloxy)-6-((benzyloxy)methyl)-5-methylenetetrahydro-2H-pyran as a white solid.

A solution of osmium tetroxide (2.5% in t-BuOH, 200 μl) was added to a solution of (2R,3R,4S,6S)-2-(5-(benzo[b]thiophen-2-ylmethyl)-2-(benzyloxy)-4-methoxyphenyl)-3,4-bis(benzyloxy)-6-((benzyloxy)methyl)-5-methylenetetrahydro-2H-pyran (622.2 mg, 0.789 mmol) and NMO (277 mg, 2.37 mmol) in acetone (15 ml) and water (1.5 ml) at room temperature. The reaction mixture was stirred at 45° C. for 16 h. The solvent was evaporated and the resulting residue was quenched with sodium metabisulfite, diluted with water, and extracted with EtOAc three times. The organic layer was washed with brine, dried with Na₂SO₄, filtered and the solvent concentrated under reduced pressure. The resulting residue was purified by flash column chromatography on silica gel (12 g, EtOAc/heptane; 0>>>10%>>>30%) to yield (2R,4R,5S,6R)-6-(5-(benzo[b]thiophen-2-ylmethyl)-2-(benzyloxy)-4-methoxyphenyl)-4,5-bis(benzyloxy)-2-((benzyloxy)methyl)-3-(hydroxymethyl)tetrahydro-2H-pyran-3-ol as a white solid.

(2R,4R,5S,6R)-6-(5-(benzo[b]thiophen-2-ylmethyl)-2-(benzyloxy)-4-methoxyphenyl)-4,5-bis(benzyloxy)-2-((benzyloxy)methyl)-3-(hydroxymethyl)tetrahydro-2H-pyran-3-ol (322.3 mg, 0.392 mmol) was dissolved in DCM (10 mL). To the resulting mixture was then added phenyl-I3-iodanediyl diacetate (193.1 mg, 0.59 mmol) and the mixture was stirred at room temperature for 16 h. The solvent was removed under reduced pressure and the resulting residue was purified by flash column chromatography on silica gel (12 g column, EtOAc/heptane: 0>>>5%>>>10%) to yield (2R,4R,5S,6R)-6-(5-(benzo[b]thiophen-2-ylmethyl)-2-(benzyloxy)-4-methoxyphenyl)-4,5-bis(benzyloxy)-2-((benzyloxy)methyl)dihydro-2H-pyran-3(4H)-one as a white solid.

Bis-(2-methoxyethyl)amino sulfur trifluoride (0.12 ml, 0.67 mmol) was added dropwise to (2R,4R,5S,6R)-6-(5-(benzo[b]thiophen-2-ylmethyl)-2-(benzyloxy)-4-methoxyphenyl)-4,5-bis(benzyloxy)-2-((benzyloxy)methyl)dihydro-2H-pyran-3(4H)-one (220 mg, 0.28 mmol) in DCM (5 mL) (in a 50 ml plastic bottle) and the resulting mixture was stirred for 16 h at room temperature. Aqueous NaHCO₃ solution was slowly added to the resulting mixture. The organic layer was separated and the aqueous layer was extracted with DCM three times. The combined organic layer was washed with brine, dried with Na₂SO₄, filtered, and concentrated to yield a yellow syrup, which was purified by flash column chromatography on silica gel (40 g column, Teledyne ISCO/Combiflash, EtOAc/heptane: 0>>>10%) to yield (2R,4R,5S,6R)-6-(5-(benzo[b]thiophen-2-ylmethyl)-2-(benzyloxy)-4-methoxyphenyl)-4,5-bis(benzyloxy)-2-((benzyloxy)methyl)-3,3-difluorotetrahydro-2H-pyran as a white foam.

(2R,4R,5S,6R)-6-(5-(benzo[b]thiophen-2-ylmethyl)-2-(benzyloxy)-4-methoxyphenyl)-4,5-bis(benzyloxy)-2-((benzyloxy)methyl)-3,3-difluorotetrahydro-2H-pyran (146.8 mg, 0.18 mmol) and 1,2,3,4,5-pentamethylbenzene (267.7 mg, 1.81 mmol) were dissolved in DCM (4 mL), and the resulting mixture was cooled to −78° C. To the mixture was then added BCl₃ (0.90 mL, 1M in DCM) dropwise, under argon, and the resulting pink mixture was stirred at −78° C. for 1.5 h, then quenched with MeOH (5 ml). The solvent was removed under reduced pressure and the resulting residue was purified by flash column chromatography on silica gel (12 g, EtOAc/heptane: 0>>>10%>>>100%) to yield the title compound—(2S,3R,4R,6R)-2-(5-(benzo[b]thiophen-2-ylmethyl)-2-hydroxy-4-methoxyphenyl)-5,5-difluoro-6-(hydroxymethyl)tetrahydro-2H-pyran-3,4-diol—as a white solid.

¹H NMR (METHANOL-d₄) δ: 7.69 (d, J=8.1 Hz, 1H), 7.61 (d, J=7.6 Hz, 1H), 7.15-7.28 (m, 3H), 6.96 (s, 1H), 6.48 (s, 1H), 4.67 (d, J=9.1 Hz, 1H), 4.04-4.13 (m, 3H), 3.73-3.88 (m, 6H). MS(ESI⁺, m/z): [M+Na]⁺ m/z 475.05

Additional representative compounds of the present invention, detailed in Table 5, below, were similarly prepared according to the procedures described in Examples 6-16 above, selecting and substituting suitably substituted reagents and starting materials, as would be readily recognized by those skilled in the art.

TABLE 5
Representative Compounds of Formula (I)

| ID No. | Compound name/Structural formula/Analysis data |
|---|---|
| 89 | (2S,3R,4R,6R)-2-[4-ethyl-2-methoxy-5-[(4- |
| | methoxyphenyl)methyl]phenyl]-5,5-difluoro-6- |
| | (hydroxymethyl)tetrahydropyran-3,4-diol |
| | ¹H NMR (METHANOL-d₄) δ: 7.16 (s, 1H), 7.01 (br d, J = 8.6 Hz, 2H), |
| | 6.74-6.84 (m, 3H), 4.75 (br d, J = 9.1 Hz, 1H), 3.68-3.92 (m, 13H), 2.56 |
| | (q, J = 7.2 Hz, 2H), 1.01-1.10 (m, 3H). MS(ESI⁺, m/z): [M + NH₄]⁺ m/z |
| | 456.15 |
| 96 | (2S,3R,4R,6R)-2-[5-[[4-(difluoromethoxy)phenyl]methyl]-2-hydroxy-4- |
| | methyl-phenyl]-5,5-difluoro-6-(hydroxymethyl)tetrahydropyran-3,4-diol |
| | ¹H NMR (METHANOL-d₄) δ: 7.12-7.19 (m, 3H), 7.03 (d, J = 8.6 Hz, |
| | 2H), 6.55-6.95 (m, 2H), 4.73 (d, J = 9.1 Hz, 1H), 3.74-3.96 (m, 7H), |
| | 2.11 (s, 3H). MS(ESI⁺, m/z): [M + Na]⁺ m/z 469.20 |
| 108 | (2S,3R,4R,6R)-5,5-difluoro-2-[2-hydroxy-5-[(4- |
| | methoxyphenyl)methyl]-4-methyl-phenyl]-6- |
| | (hydroxymethyl)tetrahydropyran-3,4-diol |
| | ¹H NMR (MeOH) δ: 7.13 (s, 1H), 7.03 (br d, J = 8.6 Hz, 2H), 6.77-6.84 |
| | (m, 2H), 6.66 (s, 1H), 4.71 (br d, J = 9.1 Hz, 1H), 3.73-3.95 (m, 10H), |
| | 2.11 (s, 3H). MS(ESI⁺, m/z): [M + Na]⁺ m/z 433.20 |

### Example 18: Compound #23

### (2S,3R,4R,5S,6R)-5-fluoro-2-(5-(4-(fluoromethoxy)benzyl)-2-hydroxy-4-methylphenyl)-6-(hydroxymethyl)-5-methyl-tetrahydro-2H-pyran-3,4-diol

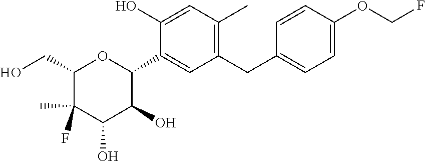 [SMILES: Cc1cc(O)c([C@H]2O[C@@H](CO)[C@](C)(F)[C@@H](O)[C@@H]2O)cc1Cc1ccc(OCF)cc1]

To a mixture of 1-bromo-4-(fluoromethoxy)benzene (3 g, 14.63 mmol, 1.00 equiv) in THF (30 mL) was added n-BuLi (5.85 mL, 14.63 mmol, 1.00 equiv, 2.5M in hexane) dropwise at −78° C., and the mixture was stirred for 30 mins at −78° C. 4-(Benzyloxy)-5-bromo-2-methyl benzaldehyde (4.46 g, 14.61 mmol, 1.00 equiv) in tetrahydrofuran (10 mL) was then added to the solution. The reaction mixture was stirred at −78° C. for 2h. NH₄Cl/H₂O was added and the mixture was extracted with EtOAc thrice. The combined extracts were washed with water twice and dried over Na₂SO₄. The mixture was concentrated and the resulting residue purified by chromatography on silica gel (3:1 PE/EA) to yield (4-(benzyloxy)-5-bromo-2-methylphenyl)(4-(fluoromethoxy)phenyl)methanol as a yellow oil.

To a mixture of (4-(benzyloxy)-5-bromo-2-methylphenyl)(4-(fluoromethoxy)phenyl)methanol (4 g, 9.27 mmol, 1.00 equiv) in dichloromethane (40 mL) with Et₃SiH (3.2 g, 27.52 mmol, 3.00 equiv) was added TMSOTf (4.1 g, 18.44 mmol, 2.00 equiv) dropwise at −78° C. The reaction mixture was stirred for 1h at −78° C. NaHCO₃/H₂O was added and the mixture was extracted with DCM thrice. The combined extracts were washed with brine and dried over Na₂SO₄. The mixture was concentrated and the resulting residue purified by chromatography on silica gel (4:1 PE/EA) to yield 1-(benzyloxy)-2-bromo-4-(4-(fluoromethoxy)benzyl)-5-methylbenzene as a yellow oil.

To a mixture of 1-(benzyloxy)-2-bromo-4-(4-(fluoromethoxy)benzyl)-5-methylbenzene (2 g, 4.82 mmol, 1.00 equiv) in THE (25 mL) was added n-BuLi (1.93 mL, 4.82 mmol, 1.00 equiv, 2.5M in hexane) dropwise at −78° C., and the mixture was stirred for 20 mins at −78° C. (3R,4S,6S)-3,4-Bis(benzyloxy)-6-[(benzyloxy)methyl]-5-methylideneoxan-2-one (2.14 g, 4.82 mmol, 1.00 equiv) in tetrahydrofuran (5 mL) was then added to the solution. The reaction mixture was stirred at −78° C. for 2h. NH₄Cl/H₂O was added and the mixture was extracted with EtOAc thrice. The combined extracts were washed with brine and dried over Na₂SO₄. The mixture was concentrated and the resulting residue purified by chromatography on silica gel (3:1 PE/EA) to yield (3R,4S,6S)-3,4-bis(benzyloxy)-2-(2-(benzyloxy)-5-(4-(fluoromethoxy)benzyl)-4-methylphenyl)-6-((benzyloxy)methyl)-5-methylenetetrahydro-2H-pyran-2-ol as a yellow oil.

To a mixture of (3R,4S,6S)-3,4-bis(benzyloxy)-2-(2-(benzyloxy)-5-(4-(fluoromethoxy)benzyl)-4-methylphenyl)-6-((benzyloxy)methyl)-5-methylenetetrahydro-2H-pyran-2-ol (1.5 g, 1.92 mmol, 1.00 equiv) in dichloromethane (15 mL) with Et₃SiH (670 mg, 5.76 mmol, 3.00 equiv) was added TMSOTf (853 mg, 3.84 mmol, 2.00 equiv) dropwise at −78° C. The reaction mixture was stirred for 1 h at −78° C. Sodium bicarbonate/H₂O was added and the mixture was extracted with DCM thrice. The combined extracts were washed with brine and dried over Na₂SO₄. The mixture was concentrated and the resulting residue purified by chromatography on silica gel (3:1 PE/EA) to yield (2S,3R,4S,6S)-3,4-bis(benzyloxy)-2-(2-(benzyloxy)-5-(4-(fluoromethoxy)benzyl)-4-methylphenyl)-6-((benzyloxy)methyl)-5-methylenetetrahydro-2H-pyran as a yellow oil.

To a mixture of (2S,3R,4S,6S)-3,4-bis(benzyloxy)-2-(2-(benzyloxy)-5-(4-(fluoromethoxy)benzyl)-4-methylphenyl)-6-((benzyloxy)methyl)-5-methylenetetrahydro-2H-pyran (1.2 g, 1.57 mmol, 1.00 equiv) in acetone (20 mL) and H₂O (5 mL) with NMO (551 mg, 4.71 mmol, 3.00 equiv) was added OsO₄ (319 mg, 0.03 mmol, 0.02 equiv, 2.5% in t-BuOH). The reaction mixture was stirred for 1h at 45° C. Water was added and extracted with EtOAc thrice. The combined extracts were washed with brine and dried over Na₂SO₄. The mixture was concentrated and the resulting residue purified by chromatography on silica gel (3:1 PE/EA) to yield (2R,4R,5S,6S)-4,5-bis(benzyloxy)-6-(2-(benzyloxy)-5-(4-(fluoromethoxy)benzyl)-4-methylphenyl)-2-((benzyloxy)methyl)-3-(hydroxymethyl)tetrahydro-2H-pyran-3-ol as a yellow oil.

To a mixture of (2R,4R,5S,6S)-4,5-bis(benzyloxy)-6-(2-(benzyloxy)-5-(4-(fluoromethoxy)benzyl)-4-methylphenyl)-2-((benzyloxy)methyl)-3-(hydroxymethyl)tetrahydro-2H-pyran-3-ol (1.1 g, 1.38 mmol, 1.00 equiv) in dichloromethane (15 mL) was added PhI(OAc)₂ (889 mg, 2.76 mmol, 2.00 equiv). The reaction mixture was stirred at room temperature overnight. Ice/water was added and extracted with DCM thrice. The combined extracts were washed with brine and dried over Na₂SO₄. The mixture was concentrated and the resulting residue purified by chromatography on silica gel (3:1 PE/EA) to yield (2R,4R,5S,6S)-4,5-bis(benzyloxy)-6-(2-(benzyloxy)-5-(4-(fluoromethoxy)benzyl)-4-methylphenyl)-2-((benzyloxy)methyl)dihydro-2H-pyran-3(4H)-one as a yellow oil.

To a mixture of (2R,4R,5S,6S)-4,5-bis(benzyloxy)-6-(2-(benzyloxy)-5-(4-(fluoromethoxy)benzyl)-4-methylphenyl)-2-((benzyloxy)methyl)dihydro-2H-pyran-3(4H)-one (1 g, 1.30 mmol, 1.00 equiv) in THE (15 mL) was added methyllithium (2.6 mL, 2.60 mmol, 2.00 equiv, 1M in Et₂O) dropwise at −78° C. in 10 min. The reaction mixture was stirred for 2h at −78° C. NH₄Cl/H₂O was added and the mixture was extracted with EtOAc thrice. The combined extracts were washed with brine and dried over Na₂SO₄. The mixture was concentrated and the resulting residue purified by chromatography on silica gel (3:1 PE/EA) to yield (2R,4R,5S,6S)-4,5-bis(benzyloxy)-6-(2-(benzyloxy)-5-(4-(fluoromethoxy)benzyl)-4-methylphenyl)-2-((benzyloxy)methyl)-3-methyltetrahydro-2H-pyran-3-ol as a yellow oil.

To a mixture of (2R,4R,5S,6S)-4,5-bis(benzyloxy)-6-(2-(benzyloxy)-5-(4-(fluoromethoxy)benzyl)-4-methylphenyl)-2-((benzyloxy)methyl)-3-methyltetrahydro-2H-pyran-3-ol (1 g, 1.28 mmol, 1.00 equiv) in dichloromethane (15 mL) was added DAST (2.05 g, 12.77 mmol, 10.00 equiv). The reaction mixture was stirred overnight at room temperature. Sodium bicarbonate/H₂O was added and the mixture was extracted with DCM thrice. The combined extracts were washed with brine and dried over Na₂SO₄. The mixture was concentrated and the resulting residue purified by chromatography on silica gel (3:1 PE/EA) to yield (2R,3R,4R,5S,6S)-4,5-bis(benzyloxy)-6-(2-(benzyloxy)-5-(4-(fluoromethoxy)benzyl)-4-methylphenyl)-2-((benzyloxy)methyl)-3-fluoro-3-methyltetrahydro-2H-pyran as a yellow oil.

A solution of (2R,3R,4R,5S,6S)-4,5-bis(benzyloxy)-6-(2-(benzyloxy)-5-(4-(fluoromethoxy)benzyl)-4-methylphenyl)-2-((benzyloxy)methyl)-3-fluoro-3-methyltetrahydro-2H-pyran (200 mg, 0.25 mmol, 1.00 equiv) and Pd(OH)₂/C (50 mg) in EtOAc (6 mL) and MeOH (2 mL) was stirred under an atmosphere of hydrogen for 30 min at room temperature. The solids were filtered out. The filtrate was concentrated and then purified by chromatograph on a C18 reversed phase column to yield (2S,3R,4R,5S,6R)-5-fluoro-2-(5-(4-(fluoromethoxy)benzyl)-2-hydroxy-4-methylphenyl)-6-(hydroxymethyl)-5-methyltetrahydro-2H-pyran-3,4-diol, the title compounds, as a white solid.

¹H-NMR (300 MHz, CD₃OD) δ: 7.10 (d, J=8.7 Hz, 3H), 6.98 (d, J=8.7 Hz, 2H), 6.67 (s, 1H), 5.80 (s, 1H), 5.62 (s, 1H), 4.58 (d, J=9.5 Hz, 1H), 3.90 (d, J=1.1 Hz, 2H), 3.63-3.86 (m, 5H), 2.13 (s, 3H), 1.36 (d, J=22.9 Hz, 3H); MS(ES) m/z: 423.1[M−H]⁻

### Example 19: Compound #6

### (2S,3R,4R,5S,6R)-2-(5-((2,3-dihydrobenzo[b][1,4]dioxin-6-yl)methyl)-2-hydroxy-4-methoxyphenyl)-5-fluoro-6-(hydroxymethyl)-5-methyl-tetrahydro-2H-pyran-3,4-diol

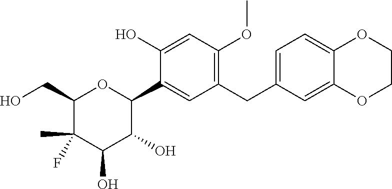 [SMILES: COc1cc(O)c([C@@H]2O[C@H](CO)[C@@](C)(F)[C@H](O)[C@H]2O)cc1Cc1ccc2c(c1)OCCO2]

To a mixture of 6-bromo-2,3-dihydrobenzo[b][1,4]dioxine (3.7 g, 17.21 mmol, 1.11 equiv) in THE (80 mL) was added n-BuLi (2.5 M in hexane, 7.5 mL, 1.20 equiv) dropwise at −78° C. The reaction mixture was stirred for 1 h at −78° C. To the reaction mixture was added a solution of 4-(benzyloxy)-5-bromo-2-methoxybenzaldehyde (5.0 g, 15.57 mmol, 1.00 equiv) in THE (20 mL) dropwise at −78° C. The reaction mixture was stirred for 3 h at −78° C. NH₄Cl/H₂O was added and the mixture was extracted with EtOAc thrice. The combined extracts were washed with brine and dried over Na₂SO₄. The filtrate was concentrated to yield (4-(benzyloxy)-5-bromo-2-methoxyphenyl)(2,3-dihydrobenzo[b][1,4]dioxin-6-yl)methanol as a yellow oil. MS (ES) m/z: 441.1 [M-OH]⁺

To a mixture of (4-(benzyloxy)-5-bromo-2-methoxyphenyl)(2,3-dihydrobenzo[b][1,4]dioxin-6-yl)methanol (7.8 g, 17.10 mmol, 1.00 equiv) in dichloromethane (60 mL) with Et₃SiH (4.0 g, 34.40 mmol, 2.02 equiv) was added CF₃CO₂H (3.93 g, 34.47 mmol, 2.02 equiv) at 0° C. The reaction mixture was then stirred for 2 h at 0° C. Sodium bicarbonate was added and the mixture was extracted with DCM thrice. The combined extracts were washed with brine and dried over Na₂SO₄. The mixture was concentrated and the resulting residue purified by chromatography on silica gel (3:1 PE/EA) to yield 6-(4-(benzyloxy)-5-bromo-2-methoxybenzyl)-2,3-dihydrobenzo[b][1,4]dioxine as a yellow oil. ¹H NMR (400 MHz, CDCl₃) δ: 7.38-7.45 (m, 2H), 7.30-7.35 (m, 2H), 7.21-7.28 (m, 1H), 7.14 (s, 1H), 6.65-6.71 (m, 1H), 6.52-6.62 (m, 2H), 6.41 (s, 1H), 5.06 (s, 2H), 4.15 (s, 4H), 3.67 (s, 5H).

To a mixture of 6-(4-(benzyloxy)-5-bromo-2-methoxybenzyl)-2,3-dihydrobenzo[b][1,4]dioxine (4.0 g, 9.06 mmol, 1.10 equiv) in tetrahydrofuran (50 mL) was then added n-BuLi (2.5 M in hexane, 4.0 mL, 10.0 mmol, 1.20 equiv) dropwise at −78° C. The solution was stirred for 1 h at −78° C. To the reaction mixture was added a solution of (3R,4S,6S)-3,4-bis(benzyloxy)-6-[(benzyloxy)methyl]-5-methylideneoxan-2-one (3.67 g, 8.26 mmol, 1.00 equiv) in tetrahydrofuran (20 mL) dropwise at −78° C. The reaction mixture was stirred for 3 h at −78° C. NH₄Cl/H₂O was added and the mixture was extracted with EtOAc twice. The combined extracts were washed with brine and dried over Na₂SO₄. The mixture was concentrated and the resulting residue purified by chromatography on silica gel (1:1 PE/EA) to yield (3R,4S,6S)-3,4-bis(benzyloxy)-2-(2-(benzyloxy)-5-((2,3-dihydrobenzo[b][1,4]dioxin-6-yl)methyl)-4-methoxyphenyl)-6-((benzyloxy)methyl)-5-methylenetetrahydro-2H-pyran-2-ol as a yellow oil. MS (ES) m/z: 852.4 [M+2Na]⁺

To a mixture of (3R,4S,6S)-3,4-bis(benzyloxy)-2-(2-(benzyloxy)-5-((2,3-dihydrobenzo[b][1,4]dioxin-6-yl)methyl)-4-methoxyphenyl)-6-((benzyloxy)methyl)-5-methylenetetrahydro-2H-pyran-2-ol (1.4 g, 1.73 mmol, 1.00 equiv) in dichloromethane (20 mL) with Et₃SiH (0.4 g, 3.44 mmol, 1.98 equiv) was added TMSOTf (0.58 g, 2.61, 1.51 equiv) dropwise at −78° C. The reaction mixture was stirred for 2 h at −78° C. Sodium bicarbonate was added and the mixture was extracted with DCM twice. The combined extracts were washed with brine and dried over Na₂SO₄. The mixture was concentrated and the resulting residue purified by chromatography on silica gel (2:1 PE/EA) to yield 6-(4-(benzyloxy)-5-((2S,3R,4S,6S)-3,4-bis(benzyloxy)-6-((benzyloxy)methyl)-5-methylenetetrahydro-2H-pyran-2-yl)-2-methoxybenzyl)-2,3-dihydrobenzo[b][1,4]dioxine as a colorless oil. MS (ES) m/z: 813.4 [M+Na]⁺

To a mixture of 6-(4-(benzyloxy)-5-((2S,3R,4S,6S)-3,4-bis(benzyloxy)-6-((benzyloxy)methyl)-5-methylenetetrahydro-2H-pyran-2-yl)-2-methoxybenzyl)-2,3-dihydrobenzo[b][1,4]dioxine (1.7 g, 2.15 mmol, 1.00 equiv) in acetone/water (35 mL/3 mL) with NMO (755 mg, 6.44 mmol, 3.00 equiv) was added OsO₄ (2.5% in t-Butanol, 0.54 mL, 0.043 mmol, 0.02 equiv). The reaction mixture was stirred overnight at room temperature. Sodium thiosulfate was added and the mixture was extracted with EtOAc twice. The combined extracts were washed with brine and dried over Na₂SO₄. The mixture was concentrated and the resulting residue purified by chromatography on silica gel (1:1 PE/EA) to yield (2R,4R,5S,6S)-4,5-bis(benzyloxy)-6-(2-(benzyloxy)-5-((2,3-dihydrobenzo[b][1,4]dioxin-6-yl)methyl)-4-methoxyphenyl)-2-((benzyloxy)methyl)-3-(hydroxymethyl)tetrahydro-2H-pyran-3-ol as a colorless oil. MS (ES) m/z: 842.3 [M+NH₄]⁺

To a mixture of (2R,4R,5S,6S)-4,5-bis(benzyloxy)-6-(2-(benzyloxy)-5-((2,3-dihydrobenzo[b][1,4]dioxin-6-yl)methyl)-4-methoxyphenyl)-2-((benzyloxy)methyl)-3-(hydroxymethyl)tetrahydro-2H-pyran-3-ol (1.57 g, 1.90 mmol, 1.00 equiv) in dichloromethane (30 mL) was added PhI(OAc)₂ (1.4 g, 4.35 mmol, 2.29 equiv). The reaction mixture was stirred overnight at room temperature. Sodium thiosulfate was added and the mixture was extracted with DCM thrice. The combined extracts were washed with brine and dried over Na₂SO₄. The mixture was concentrated and the resulting residue purified by chromatography on silica gel (2:1 PE/EA) to yield (2R,4R,5S,6S)-4,5-bis(benzyloxy)-6-(2-(benzyloxy)-5-((2,3-dihydrobenzo[b][1,4]dioxin-6-yl)methyl)-4-methoxyphenyl)-2-((benzyloxy)methyl)dihydro-2H-pyran-3(4H)-one as a light yellow oil. MS (ES) m/z: 810.3 [M+NH₄]⁺

To a mixture of (2R,4R,5S,6S)-4,5-bis(benzyloxy)-6-(2-(benzyloxy)-5-((2,3-dihydrobenzo[b][1,4]dioxin-6-yl)methyl)-4-methoxyphenyl)-2-((benzyloxy)methyl)dihydro-2H-pyran-3(4H)-one (910 mg, 1.15 mmol, 1.00 equiv) in tetrahydrofuran (18 mL) was added CH₃Li (1.0 M in ether, 3.5 mL, 3.5 mmol, 3.00 equiv) dropwise with stirring at −78° C. The reaction mixture was stirred for 2 h at −78° C. NH₄Cl/H₂O was added and the mixture was extracted with EtOAc thrice. The combined extracts were washed with brine and dried over Na₂SO₄. The mixture was concentrated and the resulting residue purified by chromatography on silica gel (2:1 PE/EA) to yield (2R,4R,5S,6S)-4,5-bis(benzyloxy)-6-(2-(benzyloxy)-5-((2,3-dihydrobenzo[b][1,4]dioxin-6-yl)methyl)-4-methoxyphenyl)-2-((benzyloxy)methyl)-3-methyltetrahydro-2H-pyran-3-ol as a light yellow oil. MS (ES) m/z: 831.5 [M+Na]⁺

To a mixture of (2R,4R,5S,6S)-4,5-bis(benzyloxy)-6-(2-(benzyloxy)-5-((2,3-dihydrobenzo[b][1,4]dioxin-6-yl)methyl)-4-methoxyphenyl)-2-((benzyloxy)methyl)-3-methyltetrahydro-2H-pyran-3-ol (740 mg, 0.91 mmol, 1.00 equiv) in dichloromethane (15 mL) was added DAST (1.5 g, 9.32 mmol, 10.2 equiv) dropwise at 0° C. The reaction mixture was stirred overnight at room temperature. Sodium bicarbonate was added and the mixture was extracted with DCM thrice. The combined extracts were washed with brine and dried over Na₂SO₄. The mixture was concentrated and the resulting residue purified by chromatography on silica gel (4:1 PE/EA) to yield 6-(4-(benzyloxy)-5-((2S,3S,4R,6R)-3,4-bis(benzyloxy)-6-((benzyloxy)methyl)-5-fluoro-5-methyltetrahydro-2H-pyran-2-yl)-2-methoxybenzyl)-2,3-dihydrobenzo[b][1,4]dioxine as a yellow oil. MS (ES) m/z: 828.3 [M+NH₄]⁺

To a mixture of 6-(4-(benzyloxy)-5-((2S,3S,4R,6R)-3,4-bis(benzyloxy)-6-((benzyloxy)methyl)-5-fluoro-5-methyltetrahydro-2H-pyran-2-yl)-2-methoxybenzyl)-2,3-dihydrobenzo[b][1,4]dioxine (470 mg, 0.58 mmol, 1.00 equiv) in methanol/EtOAc (1 mL/5 mL) was added Pd(OH)₂/C (200 mg) at room temperature, and then H₂ was introduced. The reaction mixture was stirred overnight at room temperature. The black solid was filtered out, and the organic mixture (filtrate) was concentrated and purified by prep-HPLC (10%-50% CH₃CN/H₂O) to yield (2S,3R,4R,5S,6R)-2-(5-((2,3-dihydrobenzo[b][1,4]dioxin-6-yl)methyl)-2-hydroxy-4-methoxyphenyl)-5-fluoro-6-(hydroxymethyl)-5-methyltetrahydro-2H-pyran-3,4-diol, the title compound, as a white solid.

¹H NMR (400 MHz, CD₃OD) δ: 7.00 (s, 1H), 6.61-6.68 (m, 3H), 6.45 (s, 1H), 4.50-4.53 (d, J=9.6 Hz, 1H), 4.18 (s, 4H), 3.56-3.81 (m, 10H), 1.35 (d, J=22.8 Hz, 3H); MS (ES) m/z: 468.1 [M+NH₄]⁺.

Additional representative compounds of the present invention, detailed in Table 6, below, were similarly prepared according to the procedures described in Examples 18 and 19 above, selecting and substituting suitably substituted reagents and starting materials, as would be readily recognized by those skilled in the art.

TABLE 6
Representative Compounds of Formula (I)

| ID No. | Compound name/Structural formula/Analysis data |
|---|---|
| 16 | (2S,3R,4R,5S,6R)-2-[4-ethyl-2-hydroxy-5-[(4- |
| | methoxyphenyl)methyl]phenyl]-5-fluoro-6-(hydroxymethyl)-5-methyl- |
| | tetrahydropyran-3,4-diol |
| | ¹H NMR (METHANOL-d₄) δ: 7.04 (s, 1H), 7.00 (d, J = 8.6 Hz, 2H), 6.78 |
| | (d, J = 8.6 Hz, 2H), 6.67 (s, 1H), 4.54 (d, J = 9.6 Hz, 1H), 3.85 (s, 2H), |
| | 3.56-3.82 (m, 8H), 2.48 (q, J = 7.6 Hz, 2H), 1.32 (d, J = 22.7 Hz, 3H), |
| | 1.03 (t, J = 7.6 Hz, 3H). MS(ESI⁺, m/z): [M + NH₄]⁺ m/z 438.25 |
| 3 | (2S,3R,4R,5S,6R)-2-[4-chloro-5-(2,3-dihydro-1,4-benzodioxin-6- |
| | ylmethyl)-2-hydroxy-phenyl]-5-fluoro-6-(hydroxymethyl)-5-methyl- |
| | tetrahydropyran-3,4-diol |
| | ¹H NMR (METHANOL-d₄) δ: 7.14 (s, 1H), 6.83-6.85 (m, 1H), 6.67-6.72 |
| | (m, 1H), 6.57-6.64 (m, 2H), 4.53 (d, J = 9.6 Hz, 1H), 4.17 (s, 4H), 3.51- |
| | 3.91 (m, 7H), 1.31 (d, J = 22.7 Hz, 3H). MS(ESI⁺, m/z): [M + Na]⁺ m/z |
| | 477.15 |
| 10 | (2S,3R,4R,5S,6R)-2-[5-(benzothiophen-2-ylmethyl)-4-chloro-2- |
| | hydroxy-phenyl]-5-fluoro-6-(hydroxymethyl)-5-methyl-tetrahydropyran- |
| | 3,4-diol |
| | ¹H NMR (METHANOL-d₄) δ: 7.72 (d, J = 8.1 Hz, 1H), 7.64 (d, J = 8.1 Hz, |
| | 1H), 7.32 (s, 1H), 7.20-7.29 (m, 2H), 6.98 (s, 1H), 6.88 (s, 1H), 4.56 |
| | (d, J = 9.6 Hz, 1H), 4.23-4.31 (m, 2H), 3.51-3.81 (m, 5H), 1.30 (d, |
| | J = 22.7 Hz, 3H). MS(ESI⁺, m/z): [M + Na]⁺ m/z 475.05 |
| 2 | (2S,3R,4R,5S,6R)-2-[5-(chroman-6-ylmethyl)-2-hydroxy-4-methoxy- |
| | phenyl]-5-fluoro-6-(hydroxymethyl)-5-methyl-tetrahydropyran-3,4-diol |
| | ¹H NMR (METHANOL-d₄) δ: 6.97 (s, 1H), 6.78-6.86 (m, 2H), 6.55 (d, |
| | J = 8.6 Hz, 1H), 6.42 (s, 1H), 4.49 (d, J = 9.6 Hz, 1H), 4.04-4.15 (m, 2H), |
| | 3.53-3.82 (m, 10H), 2.69 (t, J = 6.3 Hz, 2H), 1.88-1.97 (m, 2H), 1.27- |
| | 1.35 (m, 3H). MS(ESI⁺, m/z): [M + Na]⁺ m/z 471.15 |
| 9 | (2S,3R,4R,5S,6R)-2-[5-(2,3-dihydro-1,4-benzodioxin-6-ylmethyl)-2,4- |
| | dimethoxy-phenyl]-5-fluoro-6-(hydroxymethyl)-5-methyl- |
| | tetrahydropyran-3,4-diol |
| | ¹H NMR (MeOH) δ: 7.02 (s, 1H), 6.57-6.69 (m, 4H), 4.52 (d, J = 9.6 Hz, |
| | 1H), 4.16 (s, 4H), 3.83 (d, J = 5.1 Hz, 6H), 3.50-3.79 (m, 7H), 1.32 (d, |
| | J = 22.7 Hz, 3H). |
| | MS(ESI⁺, m/z): [M + Na]⁺ m/z 487.2 |

Additional representative compounds of the present invention may be similarly prepared according to the procedures described in the General Synthesis Schemes and Examples described above, selecting and substituting suitably substituted reagents and starting materials, as would be readily recognized by those skilled in the art.

### Biological Example 1: SGLT1 and SGLT2 In Vitro Assays

The ability of the compounds of formula (I) of the present invention to treat an SGLT-mediated condition was determined using the following procedures:

SGLT1 and SGLT2 were cloned in form of cDNA from human small intestine (Genbank M24847), and from human kidney (Genbank M95549), respectively. Subsequently, each full cDNA was subcloned into pcDNA with each construct's integrity verified through follow-on sequencing. To generate CHO-K1 cells that stably express human SGLT1 or human SGLT2, CHO-K1 cells were transfected using DMRIE-C reagent (Life Technologies, Gaithersburg, Md.). Transfected cells were then selected in the presence of 500 μg/ml of the Geneticin (G418 Cellgro Catalog No. 30234-CI)

Individual clones were then characterized using the following cell-based assay for sodium-dependent glucose transport:

Inhibition of SGLT1 and SGLT2 activity was assessed in CHO K1 cells stably expressing either human SGLT1 or SGLT2, using the SGLT specific glucose analog methyl-glucopyranoside (Sigma Catalog No. M-9376). Cells were plated (45,000 cells/well) in white wall 96-well plates (COSTAR, Cat #3903) for 24 hours in growth medium, then a final concentration of 10 mM Na-Butyrate (ALDRICH Cat #30341-0) was added. The cells were incubated for 24 hours. On the day of the assay, cells were rinsed and treated with test compounds (at concentrations of 0.001 μM to 10 μM) in assay buffer (50 mM HEPES, 20 mM Tris base, 5 mM KCl, 1 mM MgCl₂, 1 mM CaCl₂ and 137 mM NaCl, pH 7.4) for 10 minutes. Cells were then incubated with ¹⁴C-a-methyl-d-glucopyranoside (AMG, Amersham Catalog No. CFB 76), using 0.07 μCi per well in 500 M AMG final concentration. The cells were incubated for 2 hours at 37° C. with 5% CO₂ and washed two times with ice-cold Phosphate Buffer Solution (CELLGRO Catalog No. 21030-CV). The cells were then solubilized by adding 60 μl of MICROSCINT™ 20 and the Na-dependent ¹⁴C-AMG uptake was quantified by measuring radioactivity. Plates were counted in a TopCount (Packard, Meriden, Conn.)

Representative compounds of the present invention were tested according to the procedures as described in Biological Example 1 and 2 above, with results as listed in Table 5, below. Results are reported as the %-inhibition or IC₅₀ value. Variability for the functional assay was typically within 20%. The %-inhibition (% Inh) or IC₅₀ data were derived from the best curve fit as listed in Tables 7 and 8, below. When a listed compound was tested more than once, an average of the individual measurements is listed in Tables 7 and 8, below.

TABLE 7
SGLT1 and SGLT2 Activity
Compounds of Formula (I) where R⁰ is methyl.

| | SGLT1 IC₅₀ | SGLT1 % Inh | SGLT2 IC₅₀ | SGLT2 % Inh |
|---|---|---|---|---|
| ID No. | (μM) | @ 0.3 μM | (nM) | @ 0.3 μM |
| 1 | 0.198 | 57.6 | 19.6 | |
| 2 | | 31.1 | | 67.4 |
| 3 | 0.269 | 45.3 | 2.1 | 89.4 |
| 4 | | 0.9 | | 73.4 |
| 5 | | 21.9 | | 82.0 |
| 6 | 0.037 | 72.6 | 4.3 | 92.8 |
| 7 | | 10.8 | | 33.8 |
| 8 | | 23.6 | | 84.5 |
| 9 | | 25.3 | | 58.7 |
| 10 | 0.287 | 41.7 | 10.2 | 87.8 |
| 11 | | 14.4 | | 83.0 |
| 14 | | 36.2 | | 82.0 |
| 15 | | 19.9 | | 65.7 |
| 16 | 0.151 | 53.0 | 2.0 | 85.5 |
| 17 | | 35.2 | | 77.3 |
| 19 | | 9.3 | | 73.5 |
| 23 | 0.319 | 54.0 | 2.8 | 88.0 |
| 24 | | 29.6 | | 77.1 |

TABLE 8
SGLT1 and SGLT2 Activity
Compounds of Formula (I) where R⁰ is fluoro

| | SGLT1 IC₅₀ | SGLT1 % Inh | SGLT2 IC₅₀ | SGLT2 % Inh |
|---|---|---|---|---|
| ID No. | (μM) | @ 0.3 μM | (nM) | @ 0.3 μM |
| 25 | 0.056 | | 3.8 | |
| 26 | 0.044 | | 32.9 | |
| 27 | 0.093 | 71.3 | 4.2 | |
| 30 | 0.145 | 56.7 | 7.2 | 62.4 |
| 31 | 0.165 | | 24.1 | |
| 35 | | 39.2 | | 76.3 |
| 36 | 0.221 | 56.6 | 4.3 | 48.6 |
| 38 | | 47.4 | | 65.9 |
| 39 | | 31.8 | | 76.4 |
| 40 | 0.176 | 54.0 | 3.5 | 85.4 |
| 41 | | 5.8 | | 85.4 |
| 42 | | 8.9 | | 74.3 |
| 44 | 0.096 | 56.2 | 1.3 | 90.1 |
| 45 | 0.049 | 81.7 | 1.4 | 82.0 |
| 46 | | 18.4 | | 89.0 |
| 47 | 0.250 | 47.9 | 1.8 | 61.2 |
| 48 | | 36.5 | | 78.1 |
| 49 | | 23.2 | | 79.2 |
| 50 | | 20.4 | | 84.4 |
| 51 | | 30.8 | | 82.0 |
| 52 | 0.229 | 41.3 | 19.0 | 81.2 |
| 53 | | 0.0 | | 87.1 |
| 54 | 0.895 | 41.8 | 26.3 | 52.8 |
| 55 | | 15.8 | | 84.2 |
| 56 | 0.065 | 78.3 | 2.0 | 93.0 |
| 58 | | 36.8 | | 81.9 |
| 60 | 0.088 | 83.4 | 12.2 | 91.1 |
| 61 | | 32.2 | | 58.0 |
| 62 | 0.708 | 40.9 | 17.3 | 79.6 |
| 64 | | 6.2 | | 67.1 |
| 65 | | 16.9 | | 56.1 |
| 68 | | 26.6 | | 69.5 |
| 69 | | 64.0 | | 67.1 |
| 70 | 0.120 | 61.2 | 2.7 | 76.2 |
| 71 | | 21.6 | | 76.8 |
| 72 | | 38.5 | | 73.0 |
| 73 | | 21.4 | | 55.0 |
| 74 | | 31.1 | | 34.4 |
| 75 | | 15.5 | | 52.3 |
| 76 | 0.302 | 44.3 | 5.0 | 84.4 |
| 77 | | 22.6 | | 51.4 |
| 78 | | 28.0 | | 71.5 |
| 79 | | 20.3 | | 63.6 |
| 80 | | 31.1 | | 82.1 |
| 81 | 0.034 | 76.6 | 1.1 | 87.4 |
| 82 | 0.505 | 50.3 | 5.5 | 85.4 |
| 83 | 0.449 | 43.3 | 9.7 | 85.4 |
| 84 | | 28.5 | | 54.4 |
| 85 | | 20.8 | | 16.9 |
| 86 | | 17.4 | | 69.5 |
| 87 | | 8.4 | | 76.0 |
| 88 | 0.041 | 77.2 | 1.7 | 88.6 |
| 89 | 0.636 | 48.1 | 4.7 | 87.7 |
| 91 | 0.074 | 74.3 | 3.2 | 76.3 |
| 92 | | 30.2 | | 81.9 |
| 93 | | 34.5 | | 80.4 |
| 94 | 0.066 | 73.0 | 16.4 | 87.8 |
| 95 | | 19.5 | | 71.1 |
| 96 | 0.318 | 49.1 | 4.8 | 81.4 |
| 99 | | 17.3 | | 84.7 |
| 101 | | 30.3 | | 77.2 |
| 103 | | 26.3 | | 94.7 |
| 104 | | 39.2 | | 82.6 |
| 105 | | 38.9 | | 91.8 |
| 106 | | 29.5 | | 92.5 |
| 108 | 0.070 | 66.6 | 1.6 | 94.4 |
| 109 | | 27.4 | | 70.5 |
| 110 | 0.095 | 75.0 | 5.7 | 82.4 |
| 111 | 0.057 | 84.0 | 0.9 | 85.2 |
| 112 | 1.029 | 43.5 | 4.5 | 81.1 |
| 114 | | 36.8 | | 80.6 |
| 115 | 0.326 | 50.8 | 0.5 | 86.8 |
| 116 | 0.122 | 65.2 | 4.5 | 85.5 |
| 118 | | 17.1 | | 52.4 |
| 119 | | 24.9 | | 79.0 |
| 121 | | 20.2 | | 63.6 |
| 123 | | 30.6 | | 89.3 |
| 124 | 0.158 | 75.5 | 1.0 | 90.3 |
| 125 | | 32.9 | | 90.2 |
| 126 | | 28.7 | | 87.1 |
| 127 | 0.142 | 67.6 | 2.6 | 89.2 |
| 128 | 0.120 | 79.5 | 7.7 | 88.7 |
| 129 | | 16.4 | | 87.1 |
| 130 | | 22.9 | | 83.7 |
| 131 | 0.096 | 64.4 | 2.7 | 88.6 |
| 132 | 0.422 | 40.7 | 4.5 | 83.9 |
| 133 | 0.153 | 67.7 | 1.3 | 90.6 |
| 134 | | 31.6 | | 62.3 |
| 135 | | 16.5 | | 88.0 |
| 136 | 0.138 | 68.7 | 2.1 | 90.9 |
| 137 | | 50.4 | | 52.5 |
| 138 | 0.836 | 40.3 | 1.9 | 76.7 |
| 139 | | 16.0 | | 66.2 |
| 140 | 0.072 | 81.1 | 1.9 | 85.5 |
| 141 | 0.095 | 78.8 | 44.9 | 82.8 |
| 142 | 0.234 | 62.6 | 3.0 | 69.4 |
| 143 | | 72.3 | | 62.9 |
| 144 | | 30.6 | | 75.0 |
| 145 | 0.083 | 78.1 | 2.8 | 64.4 |
| 146 | | 41.9 | | 76.8 |
| 147 | | 26.5 | | 64.7 |
| 148 | 0.053 | 83.0 | 1.5 | 82.4 |
| 150 | 0.072 | 84.4 | 3.1 | 72.5 |
| 151 | 0.644 | 52.1 | 6.2 | 88.0 |
| 152 | | 32.7 | | 84.5 |
| 153 | 0.591 | 48.7 | 6.7 | 88.1 |
| 157 | 0.112 | 71.8 | 2.6 | 89.7 |
| 161 | 0.035 | 87.8 | 2.5 | 87.6 |
| 162 | 0.127 | 56.5 | 1.5 | 73.0 |
| 163 | 0.025 | 73.1 | 1.2 | 64.4 |
| 165 | 0.040 | 54.7 | 0.7 | 73.2 |
| 166 | | 8.5 | | 74.9 |
| 167 | 0.020 | 75.7 | 0.8 | 83.4 |
| 168 | 0.175 | 43.2 | 0.9 | 77.6 |
| 169 | | 12.1 | | 78.9 |
| 171 | 0.012 | 86.4 | 0.5 | 82.4 |
| 172 | | 40.4 | | 79.9 |
| 173 | | 17.3 | | 83.3 |
| 174 | | 38.0 | | 82.9 |
| 175 | | 24.3 | | 79.9 |
| 176 | 0.105 | 52.0 | 1.7 | 90.6 |
| 177 | 0.013 | 79.6 | 3.1 | 87.8 |
| 179 | | 24.9 | | 62.1 |
| 181 | | 6.8 | | 37.0 |
| 182 | 0.040 | 60.2 | 1.3 | 83.6 |
| 183 | | 39.3 | | 87.4 |
| 184 | | 24.7 | | 73.2 |
| 185 | | 25.4 | | 65.3 |
| 187 | 0.160 | 52.7 | 35.9 | 74.6 |
| 188 | | 21.6 | | 60.1 |
| 192 | 0.779 | 49.9 | 107.7 | 85.7 |

### Biological Example 2: In Vivo Assay

### Oral Glucose Tolerance Test (OGTT) in SD Rats

Overnight fasted SD rats were allocated into vehicle or test compound treatment groups based on their body weights (n=8 in each group). The animals were orally dosed with vehicle or test compound at a volume of 10 ml/kg at 30-minute prior to OGTT. The animals then received an OGTT challenge with 2 g/kg (50% glucose solution at the volume of 4 ml/kg, orally). The animals were immediately transferred into metabolic cages after they were dosed with the glucose. Blood glucose levels were measured at 0 (pre-OGTT), 30, 60, and 120 minutes after OGTT using one-touch Glucometer. Blood glucose excursion AUC (area under the curve of blood glucose) was determined from the blood glucose levels measured at the timepoints.

Table 9 below, presents the measured efficacy of the test compound(s), expressed as the percentage (%) of blood glucose AUC vs that in vehicle group. The data is interpreted such that, the smaller the percentage of blood glucose AUC of compound vs vehicle, the greater the efficacy of the test compound. Urine volume and urine glucose concentrations (tested by using Olympus system with Genzyme diagnostics assay reagent) were measured to determine the urine glucose excretion (UGE) in 4-hr period after OGTT (0-4 hours).

TABLE 9
Measured Blood Glucose excursion and
Urine Glucose Excretion (OGTT in SD rats)

| | dose | 120-min Blood Glucose | Urine Glucose |
|---|---|---|---|
| ID. No | (mg/kg) | AUC vs Vehicle (%) | Excretion (g/0-4 hr) |
| 30 | 0 | 100 ± 2.0 | 0.001 ± 0.0002 |
| | 1 | 86.0 ± 3.4 | 0.001 ± 0.0001 |
| | 3 | 80.3 ± 1.6 | 0.001 ± 0.0002 |
| | 10 | 72.0 ± 2.3 | 0.001 ± 0.0002 |
| 44 | 0 | 100 ± 3.4 | 0.005 |
| | 1 | 61.7 ± 2.1 | 0.002 ± 0.001 |
| | 10 | 39.3 ± 1.7 | 0.2 ± 0.02 |
| 25 | 0 | 100 ± 2.6 | 0.001 |
| | 1 | 67.8 ± 1.7 | 0.001 |
| | 10 | 36.5 ± 1.1 | 0.499 ± 0.04 |
| 6 | 0 | 100 ± 2.4 | 0.001 |
| | 1 | 71.6 ± 0.7 | 0.000 |
| | 10 | 55.5 ± 1.7 | 0.047 ± 0.008 |
| 3 | 0 | 100 ± 2.1 | 0.001 |
| | 1 | 91.3 ± 2.0 | 0.001 |
| | 10 | 69.3 ± 1.9 | 0.001 |
| 81 | 0 | 100 ± 2.6 | 0.001 |
| | 1 | 77.5 ± 2.4 | 0.099 ± 0.037 |
| | 10 | 41.0 ± 2.7 | 0.276 ± 0.032 |
| 88 | 0 | 100 ± 2.2 | 0.001 |
| | 1 | 65.9 ± 1.8 | 0.046 ± 0.014 |
| | 10 | 41.6 ± 1.1 | 0.349 ± 0.023 |
| 91 | 0 | 100 ± 1.5 | 0.001 |
| | 1 | 61.6 ± 3.9 | 0.026 ± 0.007 |
| | 10 | 41.3 ± 2.3 | 0.089 ± 0.01 |

*Data was expressed as mean ± standard error (SE), n = 8 per group; BG: blood glucose; BG AUC: area under the blood glucose curve; UGE: urine glucose excretion.

### Biological Example 3: In Vivo Assay

### SGLT Inhibition Evaluated in db/db Mice

Based on fed blood glucose levels, db/db mice were allocated into either vehicle or test compound treated groups (n=8 in each group). After grouped, the mice were orally dosed with either vehicle or test compound at 0 and 10 mg/kg, in a volume of 10 ml/kg. The mice were then immediately transferred into mouse metabolic cages, after they were dosed. Fed blood glucose levels were determined at multiple timepoints after the animals were treated. Fed blood glucose AUC (area under the curve of blood glucose) was determined from the blood glucose levels measured at the timepoints.

Table 10 below, presents the measured efficacy of the test compound(s), expressed as a percentage (%) of blood glucose AUC vs that in vehicle group. The data is interpreted such that, the smaller the percentage of blood glucose AUC of compound vs vehicle, the greater efficacy of the test compound. Total urine glucose excretion (UGE) over the 24-hr period post treatment was calculated based on urine glucose concentrations and urine volumes at multiple timepoints. Urine glucose was measured using Olympus system with Genzyme diagnostics assay reagent.

TABLE 10
Fed Blood Glucose Levels and
24-hr Urine Glucose Excretion in db/db mice

| | dose | 24-hr Blood Glucose | Urine Glucose |
|---|---|---|---|
| ID. No. | (mg/kg) | AUC vs Vehicle (%) | Excretion (g/24 hr) |
| 44 | 0 | 100 ± 2.4 | 0.27 ± 0.06 |
| | 10 | 48.4 ± 3.4 | 0.55 ± 0.12 |
| 81 | 0 | 100 ± 2.7 | Not Measured |
| | 10 | 55.0 ± 4.6 | Not Measured |

*Data was expressed as mean ± standard error (SE), n = 8 per group; BG: blood glucose; BG AUC: area under the blood glucose curve; UGE: urine glucose excretion.

### Formulation Example 1

### Solid, Oral Dosage Form—Prophetic Example

As a specific embodiment of an oral composition, 100 mg of the Compound #44 (prepared as in Example 3), Compound #6 (prepared as in Example 18) or Compound #88 (prepared as in Example 6) is formulated with sufficient finely divided lactose to provide a total amount of 580 to 590 mg to fill a size O hard gel capsule.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

Throughout this application, various publications are cited. The disclosure of these publications is hereby incorporated by reference into this application to describe more fully the state of the art to which this invention pertains.

1. A compound of formula (I) 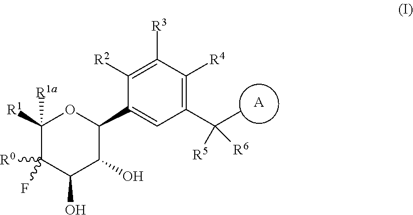
wherein
R⁰ is selected from the group consisting of fluoro and C₁₋₄alkyl;
R¹ is hydroxy substituted C₁₋₄alkyl;
R^{1a} is hydrogen;
alternatively, R¹ and R^{1a} are taken together with the carbon atom to which they are bound to form cycloprop-1,1-diyl;
R² is selected from the group consisting of hydrogen, halogen, hydroxy, C₁₋₄alkyl, —(C₁₋₄alkyl)-OH, C₁₋₄alkoxy, cyano substituted C₁₋₄alkoxy, —(C₁₋₂alkoxy)-(C₁₋₂alkoxy), C₂₋₄alkenyl, C₂₋₄alkenyl-oxy, benzyloxy and carboxy;
R³ is selected from the group consisting of hydrogen, halogen, C₁₋₄alkyl and C₂₋₄alkenyl;
R⁴ is selected from the group consisting of hydrogen, halogen, hydroxy, cyano, C₁₋₄alkyl, fluorinated C₁₋₂alkyl, C₁₋₄alkoxy, fluorinated C₁₋₂alkoxy, cyano substituted C₁₋₄alkoxy and C₃₋₆cycloalkyl;
alternatively, R² and R³ or R³ and R⁴ are taken together with the carbon atoms to which they are bound to form 2,3-dihydrofuranyl; wherein —R²—R³— is selected from the group consisting of —O—CH₂—CH₂— and —CH₂—CH₂—O; and wherein —R³—R⁴— is selected from the group consisting of —O—CH₂—CH₂— and —CH₂—CH₂—O—;
wherein the 2,3-dihydrofuranyl is optionally substituted on any of its carbon atoms with one or more substituents independently selected from the group consisting of hydroxy, methyl, ethyl, hydroxymethyl and hydroxyethyl;
R⁵ and R⁶ are the same and are each hydrogen; 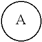
is selected from the group consisting of C₅₋₁₂cycloalkyl, C₅₋₁₂cycloalkenyl, phenyl, heteroaryl and heterocyclyl;
wherein the phenyl, heteroaryl or heterocyclyl is optionally substituted with one or more substituents independently selected from the group consisting of halogen, hydroxy, oxo, C₁₋₄alkyl, C₁₋₄alkoxy, fluorinated C₁₋₂alkyl, fluorinated C₁₋₂alkoxy, cyano, C₃₋₆cycloalkyl, tetrahydrofuranyl, phenyl, pyrid-2-yl, fluoro-substituted phenyl, fluoro-substituted pyrid-2-yl, —C(O)—R¹¹ and —NR¹²R¹³;
wherein R¹¹ is selected from the group consisting of C₁₋₄alkyl, C₁₋₄alkoxy, C₃₋₆cycloalkyl, azetidin-1-yl, pyrrolidin-1-yl, piperidin-1-yl, piperazin-1-yl, morpholin-4-yl, thiomorpholin-4-yl, thiazol-2-yl and 5-methyl-thiazol-2-yl;
wherein R¹² and R¹³ are each independently selected from the group consisting of hydrogen and C₁₋₄alkyl;
provided that when R⁰ is fluoro, R¹ is —CH₂OH, R^{1a} is hydrogen, R² is hydrogen, R³ is hydrogen, R⁴ is selected from the group consisting of chloro, fluoro, methyl, methoxy, trifluoromethyl and trifluoromethoxy, R⁵ is hydrogen and R⁶ is hydrogen,
then 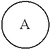
is other than phenyl, pyrid-2-yl or pyrid-3-yl; wherein the phenyl is substituted at the 4-position, the pyrid-2-yl is substituted at the 4- or 5-position and wherein the pyrid-3-yl is substituted at the 5-position with a substituent selected from the group consisting of halogen, C₁₋₄alkyl, C₁₋₄alkoxy, trifluoromethyl and trifluoromethoxy;
or an isotopologue or pharmaceutically acceptable salt thereof.

2. The compound of claim 1, wherein
R⁰ is selected from the group consisting of fluoro and C₁₋₄alkyl;
R¹ is hydroxy substituted C₁₋₄alkyl;
R^{1a} is hydrogen;
alternatively, R¹ and R^{1a} are taken together with the carbon atom to which they are bound to form cycloprop-1,1-diyl;
R² is selected from the group consisting of hydrogen, halogen, hydroxy, C₁₋₄alkyl, —(C₁₋₄alkyl)-OH, C₁₋₄alkoxy, cyano substituted C₁₋₄alkoxy, —(C₁₋₂alkoxy)-(C₁₋₂alkoxy), and carboxy;
R³ is selected from the group consisting of hydrogen, halogen and C₁₋₄alkyl;
R⁴ is selected from the group consisting of hydrogen, halogen, hydroxy, cyano, C₁₋₄alkyl, fluorinated C₁₋₂alkyl, C₁₋₄alkoxy, fluorinated C₁₋₂alkoxy and C₃₋₆cycloalkyl;
alternatively, R² and R³ or R³ and R⁴ are taken together with the carbon atoms to which they are bound to form 2,3-dihydrofuranyl; wherein —R²—R³— is selected from the group consisting of —O—CH₂—CH₂— and —CH₂—CH₂—O; wherein —R³—R⁴— is selected from the group consisting of —O—CH₂—CH₂— and —CH₂—CH₂—O—; and wherein the 2,3-dihydrofuranyl is optionally substituted on any of its carbon atoms with one or more substituents independently selected from the group consisting of hydroxy, hydroxymethyl- and hydroxyethyl-;
R⁵ and R⁶ are the same and are each hydrogen; 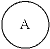
is selected from the group consisting of phenyl, heteroaryl and heterocyclyl;
wherein the phenyl, heteroaryl or heterocyclyl is optionally substituted with one or more substituents independently selected from the group consisting of halogen, hydroxy, oxo, C₁₋₄alkyl, C₁₋₄alkoxy, fluorinated C₁₋₂alkyl, fluorinated C₁₋₂alkoxy, cyano, C₃₋₆cycloalkyl, tetrahydrofuranyl, phenyl, pyrid-2-yl, fluoro-substituted phenyl, fluoro-substituted pyrid-2-yl, —C(O)—R¹¹ and —NR¹²R¹³;
wherein R¹¹ is selected from the group consisting of C₁₋₄alkyl, C₁₋₄alkoxy, C₃₋₆cycloalkyl, azetidin-1-yl, pyrrolidin-1-yl, piperidin-1-yl, piperazin-1-yl, thiazol-2-yl and 5-methyl-thiazol-2-yl;
wherein R¹² and R¹³ are each independently selected from the group consisting of hydrogen and C₁₋₄alkyl;
provided that when R⁰ is fluoro, R¹ is —CH₂OH, R^{1a} is hydrogen, R² is hydrogen, R³ is hydrogen, R⁴ is selected from the group consisting of chloro, fluoro, methyl, methoxy, trifluoromethyl and trifluoromethoxy, R⁵ is hydrogen and R⁶ is hydrogen,
then 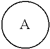
is other than phenyl, pyrid-2-yl or pyrid-3-yl; wherein the phenyl is substituted at the 4-position, the pyrid-2-yl is substituted at the 4- or 5-position and wherein the pyrid-3-yl is substituted at the 5-position with a substituent selected from the group consisting of halogen, C₁₋₄alkyl, C₁₋₄alkoxy, trifluoromethyl and trifluoromethoxy;
or an isotopologue or pharmaceutically acceptable salt thereof.

3. The compound of claim 2, wherein
R⁰ is selected from the group consisting of fluoro and C₁₋₂alkyl;
R¹ is hydroxy substituted C₁₋₂alkyl;
R^{1a} is hydrogen;
alternatively, R¹ and R^{1a} are taken together with the carbon atom to which they are bound to form cycloprop-1,1-diyl;
R² is selected from the group consisting of hydrogen, hydroxy, C₁₋₄alkoxy, —(C₁₋₂alkoxy)-(C₁₋₂alkoxy) and cyano substituted C₁₋₂alkoxy;
R³ is selected from the group consisting of hydrogen, halogen and C₁₋₂alkyl;
R⁴ is selected from the group consisting of hydrogen, halogen, cyano, C₁₋₂alkyl, C₁₋₂alkoxy and C₃₋₆cycloalkyl;
alternatively, R² and R³ or R³ and R⁴ are taken together with the carbon atoms to which they are bound to form 2,3-dihydro-furanyl; wherein —R²—R³— is selected from the group consisting of —O—CH₂—CH₂— and —CH₂—CH₂—O; wherein —R³—R⁴— is selected from the group consisting of —O—CH₂—CH₂— and —CH₂—CH₂—O—; and wherein the 2,3-dihydrofuranyl is optionally substituted on any of its carbon atoms with one to two substituents independently selected from the group consisting of hydroxymethyl- and hydroxyethyl-;
R⁵ and R⁶ are the same and are each hydrogen; 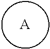
is selected from the group consisting of phenyl, thienyl, benzothienyl, 2,3-dihydrobenzofuranyl, chromanyl, 2H-benzo[b][1,4]oxazinyl, 2,3-dihydrobenzo[b][1,4]oxathiinyl, 6,7-dihydrothieno[3,2-c]pyridinyl and 2,3-dihydro-benzo[b][1,4]dioxin-6-yl;
wherein the phenyl, thienyl, benzothienyl, 2,3-dihydrobenzofuranyl, chromanyl, 2H-benzo[b][1,4]oxazinyl, 2,3-dihydrobenzo[b][1,4]oxathiinyl, 6,7-dihydrothieno[3,2-c]pyridin-2-yl or 2,3-dihydro-benzo[b][1,4]dioxin-6-yl is optionally substituted with one to two substituents independently selected from the group consisting of halogen, oxo, cyano, C₁₋₂alkyl, C₁₋₂alkoxy, fluorinated C₁₋₂alkoxy, C₃₋₅cycloalkyl, tetrahydrofuranyl, phenyl, fluoro substituted phenyl, pyrid-2-yl, fluoro substituted pyrid-2-yl, —C(O)—R¹¹ and —NR¹²R¹³;
wherein R¹¹ is selected from the group consisting of C₁₋₄alkyl, C₁₋₄alkoxy, C₃₋₆cycloalkyl, pyrrolidin-1-yl, thiazol-2-yl and 5-methyl-thiazol-2-yl;
wherein R¹² and R¹³ are each independently selected from the group consisting of hydrogen and C₁₋₂alkyl;
provided that when R⁰ is fluoro, R¹ is —CH₂OH, R^{1a} is hydrogen, R² is hydrogen, R³ is hydrogen, R⁴ is selected from the group consisting of chloro, fluoro, methyl and methoxy, R⁵ is hydrogen and R⁶ is hydrogen,
then 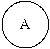
is other than phenyl; wherein the phenyl is substituted at the 4-position with a substituent selected from the group consisting of halogen, C₁₋₂alkyl and C₁₋₂alkoxy and trifluoromethoxy;
or an isotopologue or pharmaceutically acceptable salt thereof.

4. The compound of claim 3, wherein
R⁰ is selected from the group consisting of fluoro and methyl;
R¹ is hydroxymethyl-;
R^{1a} is hydrogen;
alternatively, R¹ and R^{1a} are taken together with the carbon atom to which they are bound to form cycloprop-1,1-diyl;
R² is selected from the group consisting of hydrogen, hydroxy, methoxy, ethoxy, isopropyloxy, methoxy-ethoxy- and cyano-methoxy-;
R³ is selected from the group consisting of hydrogen, bromo, iodo and methyl;
R⁴ is selected from the group consisting of hydrogen, chloro, cyano, methyl, ethyl, methoxy and cyclopropyl;
alternatively, R² and R³ or R³ and R⁴ are taken together with the carbon atoms to which they are bound to form a ring structure selected from the group consisting of 2,3-dihydro-furanyl and 3-(hydroxymethyl)-2,3-dihydrofuranyl; wherein —R²—R³— is selected from the group consisting of —CH₂—CH₂—O— and —O—CH₂—CH₂—; and wherein —R³—R⁴ is selected from the group consisting of —CH₂—CH₂—O—, —CH(CH₂OH)—CH₂—O— and —O—CH₂—CH₂—;
R⁵ and R⁶ are the same and are selected from the group consisting of hydrogen and deuterium; 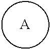
is selected from the group consisting of 4-chloro-phenyl, 3-fluoro-4-methyl-phenyl, 3-bromo-4-methoxy-phenyl, 4-methyl-phenyl, 4-ethyl-phenyl, 3-methoxy-phenyl, 4-methoxy-phenyl, 4-ethoxy-phenyl, 4-(fluoro-methoxy)-phenyl, 4-(difluoromethoxy)-phenyl, 4-(trifluoromethoxy)-phenyl, 4-(dimethylamino)-phenyl, 4-(cyclopropyl)-phenyl, 4-((S)-tetrahydrofuran-3-yl)-phenyl), 5-chloro-thien-2-yl, 5-methyl-thien-2-yl, 4-ethyl-thien-2-yl, 5-(3-fluoro-phenyl)-thien-2-yl, 5-(4-fluoro-phenyl)-thien-2-yl, 5-(6-fluoro-pyrid-2-yl)-thien-2-yl, benzothien-2-yl, 5-fluoro-benzothien-2-yl, 2,3-dihydro-benzofuran-5-yl, 2,3-dihydro-benzofuran-6-yl, chroman-6-yl, 4-methyl-2H-benzo[b][1,4]oxazin-7-yl-3-one, 2,3-dihydrobenzo[b][1,4]oxathiin-6-yl, 2,3-dihydrobenzo[b][1,4]oxathiin-6-yl-4,4-dioxide, 5-cyano-6,7-dihydrothieno[3,2-c]pyridin-2-yl, 5-(methyl-carbonyl)-6,7-dihydrothieno[3,2-c]pyridin-2-yl, 5-(methoxy-carbonyl)-6,7-dihydrothieno[3,2-c]pyridin-2-yl, 5-(cyclopentyl-carbonyl)-6,7-dihydrothieno[3,2-c]pyridin-2-yl, 5-(pyrrolidin-1-yl-carbonyl)-6,7-dihydrothieno[3,2-c]pyridin-2-yl, 5-(thiazol-2-yl-carbonyl)-6,7-dihydrothieno[3,2-c]pyridin-2-yl, 5-(5-methyl-thiazol-2-yl-carbonyl)-6,7-dihydrothieno[3,2-c]pyridin-2-yl and 2,3-dihydro-benzo[b][1,4]dioxin-6-yl;
provided that when R⁰ is fluoro, R¹ is —CH₂OH, R^{1a} is hydrogen, R² is hydrogen, R³ is hydrogen, R⁴ is selected from the group consisting of chloro, methyl and methoxy, R⁵ is hydrogen and R⁶ is hydrogen,
then 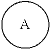
is other 4-chlorophenyl, 4-methylphenyl, 4-ethylphenyl, 4-methoxyphenyl, 4-ethoxyphenyl or 4-(trifluoromethoxy)phenyl;
or a pharmaceutically acceptable salt thereof.

5. The compound of claim 4, wherein
R⁰ is methyl;
R¹ is hydroxymethyl-;
R^{1a} is hydrogen;
R² is selected from the group consisting of hydrogen, hydroxy and methoxy;
R³ is selected from the group consisting of hydrogen and iodo;
R⁴ is selected from the group consisting of chloro, methyl, ethyl and methoxy;
alternatively, R³ and R⁴ are taken together with the carbon atoms to which they are bound to form 2,3-dihydro-furanyl; wherein —R³—R⁴— is —CH₂—CH₂—O—;
R⁵ and R⁶ are the same and are each hydrogen; 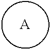
is selected from the group consisting of 4-methoxy-phenyl, 4-(fluoro-methoxy)-phenyl, benzothien-2-yl, 5-fluoro-benzothien-2-yl, chroman-6-yl and 2,3-dihydro-benzo[b][1,4]dioxin-6-yl;
or a pharmaceutically acceptable salt thereof.

6. The compound of claim 4, wherein
R⁰ is fluoro;
R¹ is hydroxymethyl-;
R^{1a} is hydrogen;
alternatively, R¹ and R^{1a} are taken together with the carbon atom to which they are bound to form cycloprop-1,1-diyl;
R² is selected from the group consisting of hydrogen, hydroxy, methoxy, ethoxy, isopropyloxy, methoxy-ethoxy- and cyano-methoxy-;
R³ is selected from the group consisting of hydrogen, bromo and methyl;
R⁴ is selected from the group consisting of hydrogen, chloro, cyano, methyl, ethyl, methoxy and cyclopropyl;
alternatively, R² and R³ or R³ and R⁴ are taken together with the carbon atoms to which they are bound to form a ring structure selected from the group consisting of 2,3-dihydro-furanyl and 3-(hydroxymethyl)-2,3-dihydrofuranyl; wherein —R²—R³— is selected from the group consisting of —CH₂—CH₂—O— and —O—CH₂—CH₂—; and wherein —R³—R⁴ is selected from the group consisting of —CH₂—CH₂—O—, —CH(CH₂OH)—CH₂—CH₂—O— and —O—CH₂—CH₂—;
R⁵ and R⁶ are the same are selected from the group consisting of hydrogen and deuterium; 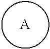
is selected from the group consisting of 4-chloro-phenyl, 3-fluoro-4-methyl-phenyl, 3-bromo-4-methoxy-phenyl, 4-methyl-phenyl, 4-ethyl-phenyl, 3-methoxy-phenyl, 4-methoxy-phenyl, 4-ethoxy-phenyl, 4-(fluoro-methoxy)-phenyl, 4-(difluoromethoxy)-phenyl, 4-(trifluoromethoxy)-phenyl, 4-(dimethylamino)-phenyl, 4-(cyclopropyl)-phenyl, 4-((S)-tetrahydrofuran-3-yl)-phenyl), 5-chloro-thien-2-yl, 5-methyl-thien-2-yl, 4-ethyl-thien-2-yl, 5-(3-fluoro-phenyl)-thien-2-yl, 5-(4-fluoro-phenyl)-thien-2-yl, 5-(6-fluoro-pyrid-2-yl)-thien-2-yl, benzothien-2-yl, 5-fluoro-benzothien-2-yl, 2,3-dihydro-benzofuran-5-yl, 2,3-dihydro-benzofuran-6-yl, chroman-6-yl, 4-methyl-2H-benzo[b][1,4]oxazin-7-yl-3-one, 2,3-dihydrobenzo[b][1,4]oxathiin-6-yl, 2,3-dihydrobenzo[b][1,4]oxathiin-6-yl-4,4-dioxide, 5-cyano-6,7-dihydrothieno[3,2-c]pyridin-2-yl, 5-(methyl-carbonyl)-6,7-dihydrothieno[3,2-c]pyridin-2-yl, 5-(methoxy-carbonyl)-6,7-dihydrothieno[3,2-c]pyridin-2-yl, 5-(cyclopentyl-carbonyl)-6,7-dihydrothieno[3,2-c]pyridin-2-yl, 5-(pyrrolidin-1-yl-carbonyl)-6,7-dihydrothieno[3,2-c]pyridin-2-yl, 5-(thiazol-2-yl-carbonyl)-6,7-dihydrothieno[3,2-c]pyridin-2-yl, 5-(5-methyl-thiazol-2-yl-carbonyl)-6,7-dihydrothieno[3,2-c]pyridin-2-yl, and 2,3-dihydro-benzo[b][1,4]dioxin-6-yl;
provided that when R⁰ is fluoro, R¹ is —CH₂OH, R^{1a} is hydrogen, R² is hydrogen, R³ is hydrogen, R⁴ is selected from the group consisting of chloro, methyl and methoxy, R⁵ is hydrogen and R⁶ is hydrogen,
then 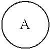
is other 4-chlorophenyl, 4-methylphenyl, 4-ethylphenyl, 4-methoxyphenyl, 4-ethoxyphenyl or 4-(trifluoromethoxy)phenyl;
or a pharmaceutically acceptable salt thereof.

7. The compound of claim 5, wherein
R⁰ is methyl;
R¹ is hydroxymethyl-;
R^{1a} is hydrogen;
R² is hydroxy;
R³ is hydrogen;
R⁴ is selected from the group consisting of chloro, methyl, ethyl and methoxy;
R⁵ and R⁶ are the same and are each hydrogen; 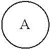
is selected from the group consisting of 4-methoxyphenyl, 4-(fluoromethoxy)-phenyl, benzothien-2-yl and 2,3-dihydro-benzo[b][1,4]dioxin-6-yl;
or a pharmaceutically acceptable salt thereof.

8. The compound of claim 4, wherein
R⁰ is selected from the group consisting of fluoro and methyl;
R¹ is hydroxymethyl-;
R^{1a} is hydrogen;
alternatively, R¹ and R^{1a} are taken together with the carbon atom to which they are bound to form cycloprop-1,1-diyl;
R² is selected from the group consisting of hydrogen, hydroxy, methoxy and cyano-methoxy-;
R³ is selected from the group consisting of hydrogen and methyl;
R⁴ is selected from the group consisting of hydrogen, chloro, methyl, ethyl, methoxy and cyclopropyl;
alternatively, R² and R³ or R³ and R⁴ are taken together with the carbon atoms to which they are bound to form 2,3-dihydro-furanyl; wherein —R²—R³— is —O—CH₂—CH₂— and wherein —R³—R⁴— is selected from the group consisting of —CH₂—CH₂—O— and —O—CH₂—CH₂—;
R⁶ and R⁶ are the same and are selected from the group consisting of hydrogen and deuterium; 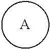
is selected from the group consisting of 4-chloro-phenyl, 3-fluoro-4-methyl-phenyl, 4-methyl-phenyl, 4-ethyl-phenyl, 3-methoxy-phenyl, 4-methoxy-phenyl, 4-ethoxy-phenyl, 4-(fluoro-methoxy)-phenyl, 4-(difluoromethoxy)-phenyl, 4-(cyclopropyl)-phenyl, 5-chloro-thien-2-yl, 5-methyl-thien-2-yl, 4-ethyl-thien-2-yl, 5-(6-fluoro-pyrid-2-yl)-thien-2-yl, benzothien-2-yl, 5-fluoro-benzothien-2-yl, 2,3-dihydro-benzofuran-5-yl, 2,3-dihydro-benzofuran-6-yl, chroman-6-yl, 2,3-dihydrobenzo[b][1,4]oxathiin-6-yl, 5-cyano-6,7-dihydrothieno[3,2-c]pyridin-2-yl, 5-(5-methyl-thiazol-2-yl-carbonyl)-6,7-dihydrothieno[3,2-c]pyridin-2-yl and 2,3-dihydro-benzo[b][1,4]dioxin-6-yl;
provided that when R⁰ is fluoro, R¹ is —CH₂OH, R^{1a} is hydrogen, R² is hydrogen, R³ is hydrogen, R⁴ is selected from the group consisting of chloro, methyl and methoxy, R⁵ is hydrogen and R⁶ is hydrogen,
then 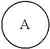
is other 4-chlorophenyl, 4-methylphenyl, 4-ethylphenyl, 4-methoxyphenyl or 4-ethoxyphenyl;
or a pharmaceutically acceptable salt thereof.

9. The compound of claim 4, wherein
R⁰ is selected from the group consisting of fluoro and methyl;
R¹ is hydroxymethyl-;
R^{1a} is hydrogen;
R² is selected from the group consisting of hydroxy, methoxy and cyano-methoxy-;
R³ is hydrogen;
R⁴ is selected from the group consisting of hydrogen, chloro, methyl, ethyl and methoxy;
R⁵ and R⁶ are the same and are each hydrogen; 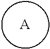
is selected from the group consisting of 4-chloro-phenyl, 3-fluoro-4-methyl-phenyl, 4-methyl-phenyl, 4-ethyl-phenyl, 3-methoxy-phenyl, 4-methoxy-phenyl, 4-ethoxy-phenyl, 4-(fluoro-methoxy)-phenyl, 4-(difluoromethoxy)-phenyl, 4-(cyclopropyl)-phenyl, 5-chloro-thien-2-yl, 5-methyl-thien-2-yl, 4-ethyl-thien-2-yl, 5-(6-fluoro-pyrid-2-yl)-thien-2-yl, benzothien-2-yl, 2,3-dihydro-benzofuran-6-yl, chroman-6-yl and 2,3-dihydro-benzo[b][1,4]dioxin-6-yl;
or a pharmaceutically acceptable salt thereof.

10. The compound of claim 4, wherein
R⁰ is selected from the group consisting of fluoro and methyl;
R¹ is hydroxymethyl-;
R^{1a} is hydrogen;
R² is selected from the group consisting of hydroxy and methoxy;
R³ is hydrogen;
R⁴ is selected from the group consisting of chloro, methyl, ethyl and methoxy;
R⁵ and R⁶ are the same and are each hydrogen; 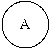
is selected from the group consisting of 4-chloro-phenyl, 4-methyl-phenyl, 4-methoxy-phenyl, 4-ethoxy-phenyl, 4-(fluoro-methoxy)-phenyl, 4-(cyclopropyl)-phenyl, 5-chloro-thien-2-yl, 5-methyl-thien-2-yl, 4-ethyl-thien-2-yl, benzothien-2-yl, 2,3-dihydro-benzofuran-6-yl, chroman-6-yl and 2,3-dihydro-benzo[b][1,4]dioxin-6-yl;
or a pharmaceutically acceptable salt thereof.

11. The compound of claim 4, wherein the compound is selected from the group consisting of
(2S,3R,4R,5S,6R)-2-(5-((2,3-dihydrobenzo[b][1,4]dioxin-6-yl)methyl)-2-hydroxy-4-methoxyphenyl)-5-fluoro-6-(hydroxymethyl)-5-methyl-tetrahydro-2H-pyran-3,4-diol;
(2S,3R,4R,6R)-2-(5-(benzo[b]thiophen-2-ylmethyl)-2-hydroxy-4-methylphenyl)-5,5-difluoro-6-(hydroxymethyl)tetrahydro-2H-pyran-3,4-diol;
(2S,3R,4R,6R)-2-[5-(benzothiophen-2-ylmethyl)-2-hydroxy-4-methoxy-phenyl]-5,5-difluoro-6-(hydroxymethyl)tetrahydropyran-3,4-diol;
(2S,3R,4R,6R)-2-(5-(chroman-6-ylmethyl)-2-hydroxy-4-methoxyphenyl)-5,5-difluoro-6-(hydroxymethyl)tetrahydro-2H-pyran-3,4-diol;
(2S,3R,4R,6R)-2-(5-((2,3-dihydrobenzo[b][1,4]dioxin-6-yl)methyl)-2-hydroxy-4-methylphenyl)-5,5-difluoro-6-(hydroxymethyl)tetrahydro-2H-pyran-3,4-diol;
(2S,3R,4R,6R)-2-(4-chloro-2-hydroxy-5-(4-methoxybenzyl)phenyl)-5,5-difluoro-6-(hydroxymethyl)-tetrahydro-2H-pyran-3,4-diol;
(2S,3R,4R,6R)-2-(4-ethyl-2-hydroxy-5-(4-methoxybenzyl)phenyl)-5,5-difluoro-6-(hydroxymethyl)-tetrahydro-2H-pyran-3,4-diol;
(2S,3R,4R,6R)-5,5-difluoro-2-(2-hydroxy-4-methoxy-5-(4-methoxybenzyl)phenyl)-6-(hydroxymethyl)-tetrahydro-2H-pyran-3,4-diol;
(2S,3R,4R,6R)-2-(4-chloro-5-((2,3-dihydrobenzofuran-6-yl)methyl)-2-hydroxyphenyl)-5,5-difluoro-6-(hydroxymethyl)-tetrahydro-2H-pyran-3,4-diol;
and isotopologues and pharmaceutically acceptable salts thereof.

12. A pharmaceutical composition comprising a pharmaceutically acceptable carrier and a compound of claim 1.

13-14. (canceled)

15. A method of treating a disorder mediated by SGLT activity, comprising administering to a subject in need thereof a therapeutically effective amount of the compound of claim 1.

16. The method of claim 15, wherein the disorder mediated by SGLT activity is selected from the group consisting of impaired glucose tolerance (IGT), impaired fasting glucose (IFT), gestational diabetes, Type II diabetes mellitus, Syndrome X, obesity, nephropathy, neuropathy, retinopathy, hypertension, angina, atherosclerosis, heart disease, heart attack, ischemia, stroke, nerve damage or poor blood flow in the feet, non-alcoholic steatohepatitis (NASH), non-alcoholic fatty liver disease (NAFLD), liver fibrosis, cataracts, polycystic ovarian syndrome, irritable bowel syndrome, inflammation and cancer.

17. The method of claim 15, wherein the disorder mediated by SGLT activity is selected from the group consisting of impaired glucose tolerance, impaired fasting glucose, Type II Diabetes Mellitus, obesity, nephropathy, neuropathy, retinopathy, atherosclerosis, hypertension, heart disease, ischemia, stroke, non-alcoholic steatohepatitus (NASH), non-alcoholic fatty liver disease (NAFLD), liver fibrosis, cataracts, polycystic ovarian syndrome, irritable bowel disorder, inflammation, prostate cancer and pancreatic cancer.

18. The method of claim 15, wherein the disorder mediated by SGLT activity is selected from the group consisting of impaired glucose tolerance, impaired fasting glucose, Type II Diabetes Mellitus, obesity, nephropathy, neuropathy, retinopathy, atherosclerosis, hypertension, heart disease, ischemia, stroke, non-alcoholic steatohepatitis (NASH), non-alcoholic fatty liver disease (NAFLD) and liver fibrosis.

19-26. (canceled)
